(19) United States
(12) Patent Application Publication
(10) Pub. No.: US 2023/0323565 A1
(43) Pub. Date: Oct. 12, 2023

(54) ARRAY, SOAKING SOLUTIONS AND METHOD OF SELECTING SOAKING CONDITIONS FOR SMALL MOLECULES IN BIOLOGICAL MACROMOLECULAR CRYSTALS

(71) Applicant: CRYSTALSFIRST GMBH, Marburg (DE)

(72) Inventors: Gerhard KLEBE, Marburg (DE); Stefan MERKL, Marburg (DE); Serghei GLINCA, Baunatal (DE); Kan FU, Marburg (DE)

(73) Assignee: CRYSTALSFIRST GMBH, Marburg (DE)

(21) Appl. No.: 18/025,107

(22) PCT Filed: Sep. 8, 2021
(86) PCT No.: PCT/EP2021/074724

§ 371 Date: Mar. 7, 2023

(30) Foreign Application Priority Data
Sep. 8, 2020 (EP) 20195080.5

(51) Int. Cl. C40B 40/04; C30B 7/14

(52) U.S. Cl. CPC C40B 40/04; C30B 7/14

(57) ABSTRACT

Subject-matter of the present invention is an array comprising soaking solutions for soaking a biological macromolecular crystal. Further, the subject of the invention is a rule-based method of selecting specific soaking solution compositions having a specific composition comprising composite solute(s), water (w), crystallization solution (crs) and/or organic solvent(s). Additionally, the subjects matter of the invention the soaking solutions obtained by the method of the invention and a screening method for small molecules comprising molecular probes, fragments and drug-size molecules using the soaking solutions and the use of the soaking solutions in a screening method for small molecules on a macromolecular crystal.

Subject-matter of the present invention is an array comprising soaking solutions for soaking a biological macromolecular crystal. Further, the subject of the invention is a rule-based method of selecting specific soaking solution compositions having a specific composition comprising composite solute(s), water (w), crystallization solution (crs) and/or organic solvent(s). Additionally, the subjects matter of the invention the soaking solutions obtained by the method of the invention and a screening method for small molecules comprising molecular probes, fragments and drug-size molecules using the soaking solutions and the use of the soaking solutions in a screening method for small molecules on a macromolecular crystal.

### BACKGROUND

Traditional methods of identifying new drug motifs in drug discovery use combinatorial chemistry and high throughput screening (HTS). This has resulted in chemical libraries of continuously increasing size and complexity, without concomitantly producing increasing numbers of lead structures. The size of the drug-like chemical universe appears to be too vast for any in vitro and in silico screening approach. A way out of this dilemma is the use of small molecules called fragments for screening. Fragments are smaller in size and weaker in both binding and activity than typical drug-size compounds. Nevertheless, fragments are sufficiently diverse and information-rich as to provide for straightforward compound development strategies.

The fragments need to be screened with techniques suitable for the detection of presumably weak interactions. Guided by such early stage structure affinity information, which is determined by i.e. the dissociation constant KD, small molecules of higher affinity can be assembled from a combination of low affinity binders or an optimization of individual low affinity binders.

Soaking is an important technique to use crystals from biological macromolecules to produce complexes from respective crystals and small molecules in crystallography-based screenings. According to the prior art a biological macromolecular crystal will be soaked in a concentrated small molecule solution. Depending on the affinity of the molecules, different concentrations of small molecule solutions are used. For low affinity molecules a higher concentrated small molecule solution is used, whereas lower concentrations of the small molecule solution can be used for high affinity molecules. During the soaking, the small molecules, which include molecular probes, fragments and drug-size molecules, will diffuse into the biological macromolecular crystal and will bind to the binding sites of the biological macromolecule. According to the prior art, the crystallization buffer/solution of the crystallized biological macromolecule is used as soaking solution after extensive optimization. Typically, such soaking solutions comprise organic solvents such as DMSO or methanol. Normally, such organic solvents as well as small molecules and compatible solutes (osmolytes having cryoprotective properties) have a disadvantageous effect on the crystals. Notably, crystallographic measurements are either conducted at cryogenic temperatures such as at 100 K or less, or at room temperature. Crystallographic measurements using diffraction techniques (xray-, electron-, neutron diffraction) at cryogenic temperatures require the use of soaking solutions with cryoprotective features to keep an intact structure of the biological macromolecular crystal.

### PRIOR ART

Traditional methods of identifying new small molecule-drug motifs in drug discovery usually use high throughput screenings (HTS), biophysical screening cascades, phenotypic screenings, screenings on DNA-encoded libraries (DEL) etc. This has resulted in chemical libraries comprising continuously increasing numbers of small molecules, without concomitantly producing increasing numbers of lead structures. The size of the drug-like chemical universe appears to be too vast for any exhaustive in vitro and in silico screening approach. A way out of this dilemma is the use of small molecules called fragments for screening. Fragments are smaller in size and weaker in both binding and activity than typical lead-like or drug-like compounds. Nevertheless, fragments are suitable starting points for drug discovery campaigns since, once identified or validated crystallographically, they can be easily chemically modified and extended according to the geometry and biophysical setup of a protein's binding pocket. A successful extension of fragments to more drug-size compounds results in tailor-made compounds of desired affinity and molecular properties. A fragment approach proves to be suitable even in case of challenging and so called “undruggable” targets. Fragments need to be screened using techniques with suitable sensitivity for the detection of presumably weak molecular interactions. In this respect, crystallographic screening approaches are unsurpassed in terms of their sensitivity towards low affinity binders. They can discriminate binders from non-binders with very low false positive and false negative rates and, at the same time, elucidate the atomically resolved geometry of the interactions of proteins and binders.

Crystallography-based screenings for small molecules include the crystallization of biological macromolecules, especially proteins, soaking of respective crystals of biological macromolecules in solutions of small molecules, subsequent data collection using x-ray or neutron diffraction and, finally, examination of the obtained data sets in order to identify binders and elucidate binding modes. The so called “soaking” is an important technique to produce such protein-small molecule complexes. According to prior art, the experimenter usually proceeds in two steps in order to derive such soaking solution compositions. These two steps are the soaking experiment itself and the cryo-preservation of the soaked crystals.
1. The soaking experiment: For the soaking experiment the experimenter transfers according crystals from their original crystallization solution to a soaking solution. The soaking solution is usually based on the crystallization solution/buffer (or reservoir solution if the vapor diffusion method is used to produce crystals) but differs from the crystallization solution due to the presence of organic solvents that are required to dissolve and introduce small molecules targeting the biological macromolecules.
2. The cryo-preservation: For cryo-preservation according crystals are transferred from the soaking solution to a cryo-preservation solution that is usually based on the crystallization solution (or reservoir solution if the vapor diffusion method is used to produce crystals) but differs from the crystallization solution and the soaking solution due to the presence of cryoprotectants such as glycerol or polyethylene glycols and a lack of organic solvents. The cryoprotectant inhibits the appearance of water in a crystalline state (ice) in or around the crystals upon vitrification in, for example, liquid nitrogen. Vitrification is generally used in order to perform diffraction experiments at cryogenic temperatures, typically 100 K or less, in order to prevent the crystals from radiation damage in the course of the high-energetic x-ray measurements. The appearance of ice must be prevented since it can destroy the crystals and the diffraction of ice crystals superimposes the diffraction patterns of the protein crystals leading to quality loss in the obtained data sets. Measurements at room temperature are performed less often. In some cases, a special cryoprotection step is not needed due to the use of a cryoprotecting additive in the crystallization. Sometimes experts in crystallography add an amount of cryoprotectant already in the first of these two steps and leave the second step out. Like the rest of the process, this is done purely on a trial and error basis without any systematic reasoning.

If, finally, a suitable soaking and cryo-preservation solution is obtained, the actual soaking experiments are performed in the presence of a set of small molecules such as small molecule fragments. This comprises the transfer of protein crystals from the original crystallization environment (a crystallization solution) to a soaking environment, which introduces small molecules of interest dissolved in an organic solvent. Small molecules of low affinity require higher concentrations, whereas molecules of high affinity require lower concentrations of the small molecule in these solutions. In order to increase hit rates in the screenings the expert strives for highly concentrated solutions of small molecules. Since macromolecular crystals, especially proteins, in contrast to crystals of low-molecular-weight compounds, are traversed by solvent channels small molecules can diffuse easily and within seconds through crystals of adequate solvent channel size. In order to increase hit rates even for low affinity binders the expert strives for long soaking times (minutes, hours, days) since even low affinity binders can be extended towards high affinity binders by chemical modifications aiming for drug-like properties guided by medicinal chemistry. In a screening, during this soaking time the small molecules (including small molecules of different molecular weight as molecular probes, fragments, drug-size molecules) will diffuse into the macromolecule crystal, especially protein crystal, and will bind or will not bind to according binding sites. A binding site is any location of the macromolecule that builds direct or solvent mediated interactions with a respective small molecule. As drug-size small molecule-concentration a concentration of 5-20 mM or more may be used. In case of fragment-concentration concentrations of 10-50 mM or more may be used. For screening purposes in both cases concentrations as high as possible (that is: without detrimental effects on the crystals) are desired (up to 1 M). However, as discussed below, one problem associated with the addition of high concentrations of small molecules dissolved in organic solvent is the impact on the stability of the crystal.

Since crystals of biological macromolecules, especially protein crystals, are very sensitive to any change in their environments, they most often exhibit a severe loss of quality or are even destroyed when they are transferred from an original crystallization solution to a soaking solution. In contrast to the original crystallization solutions, soaking solutions contain components as organic solvents and small molecules and, therefore, a soaking solution differs from the composition of the original growth/crystallization solution. Since such differences exert detrimental effects on crystals of biological macromolecules, successful crystallographic screenings require extensive optimizations of such soaking solutions in order to compensate for according detrimental effects on the crystals of biological macromolecules. According to the prior art the optimization is focused on the original crystallization solution (or reservoir solution if the crystals are produced according to the vapor diffusion-method) and requires an extensive and unsystematic trial and error search for a unique suitable soaking solution compositions that is tolerated by the individual type of crystals. Usually, the experimenter uses some kind of experimental plates in which he prepares soaking solutions that are based on the original crystallization solution (or reservoir buffer) but contain an organic solvent such as DMSO for solubilizing small molecule fragments. If the crystallization method is the vapor diffusion method, the crystallography expert usually uses a reservoir buffer in the reservoir of the experimental plates in order to maintain suitable osmotic conditions. In the approach of the prior art, the optimization is performed by applying on all possible parameters and in particular a fine-tuning of the components of the used crystallization or reservoir solution, pH-adjustments, introduction of additional additives (sometimes even cryoprotectants), changes in compositions and volume ratios between reservoir buffer and soaking solution etc. and fine-tuning of the amount of organic solvent in a trial and error manner. Especially, the presence of organic solvents exerts detrimental effects on the macromolecular crystals. Therefore, this results in typically low amounts of organic solvents, typically DMSO, between 0.5% and 5%. The result is usually a single soaking solution composition that is tolerated by the crystals. After the soaking, the already mentioned cryo-preservation takes place. For the purpose of cryoprotection, again, the solution must be optimized in order to make it tolerable for the soaked crystals. This means to introduce variations in concentrations of a variety of cryoprotectants and further adjustments as outlined above.

This approach can result in soaking conditions that reduce quality loss. However, since only one soaking condition is identified, it must be used for every soaking experiment which does not ensure best possible results in terms of hit rates, crystal diffraction and other quality parameters as mosaicity. Moreover, in the vast majority of cases, crystals of biological macromolecules are measured at cryogenic temperatures, typically 100 K or less, in order to prevent radiation damage. Measurements at room temperature are performed less often. Measurement at cryogenic temperatures requires crystal treatment using cryoprotectants and subsequent vitrification in, for example, liquid nitrogen. Again, this step of applying cryoprotective agents has a disadvantageous effect on the crystals making a re-optimization obligatory.

The pivotal point of the method and array of the present invention consists in the use of a systematic and rule-based variation of limited but critical parameters that affect crystal stability. The array and the method of the invention results in the selection and identification of not just one soaking solution, a range of suitable conditions that maintains and even improves crystal quality and can be utilized to compensate even for small molecule-induced effects like shifts in pH etc. A further pivotal point is that the array and the method of the invention eliminate the need to use the two distinct steps, that is, of the soaking experiment itself and the cryo-preservation. Moreover, the invention introduces a systematic rule-based process to infer suitable soaking solution compositions without the need for time-consuming, unsystematic trail & error dependent optimization steps. Using an A/B gradient algorithm to play off the general components of crs, w, os, and cs in order to find a small molecule soaking solution that is equivalent to the original crystallization solution in terms of osmotic net pressure, solubility of the biological macromolecule (protein), permittivity, and structural conformation where os and cs are considered to be antagonists in respect to the proteins structural conformation. Indeed, the array and the method of the invention considers the hitherto unconsidered fact that organic solvents and a class of additives known as compatible solutes compensate for detrimental effects that both components exert if used in distinct steps as in the prior art. This requires a balancing of the respective concentration of both components and very often an additional balancing to a buffer that maintains some degree of ionic strength. Furthermore, the invention allows for identifying soaking solution compositions of higher amounts of organic solvents, typically DMSO at more than 5%, preferably 10%. Advantageously, the method of the invention also often allows for very short or on the contrary extended soaking times of many hours, typically 6 to 48 hours, instead of oftentimes just seconds and minutes thus substantially improving the efficacy of the small molecule screening on the crystal. The invention thus gives more possibilities to the expert and flexibility in the setup of the small molecule screening process. In addition, in contrast with the approach of the prior art, the application of a reservoir buffer which may oftentimes be even detrimental is not required

Finally, the present invention allows for the determination of new soaking solutions that aim at reducing disorder in biological macromolecular crystals. Oftentimes, especially proteins contain parts of the amino acid chain that do not adopt fixed spatial configurations but are disordered. Such parts of the structure result in blurred electron densities that are difficult to subject to automated data processing. Rule-based method of the invention allows compatible solutes to be substantially increased which then results in stabilization of such disordered region. Therefore, using the rule-based approach of the method of the invention, the volume of compatible solutes can be substantially increased while maintaining the required adjustment and balancing of other essential components such as organic solvents in the suitable soaking solution where the crystal structure is preserved and screening method of small molecule binder can be effectively performed on the crystal.

The subject matter of the invention thus gives more possibilities to the expert and flexibility in the setup of the small molecule screening process. In addition, in contrast with the approach of the prior art, the application of a reservoir buffer which may be oftentimes even detrimental is not required.

Finally, in general, it is believed that macromolecular crystals from every individual protein species require individual soaking solution compositions that are based on their original crystallization condition. One aspect of the present invention actually demonstrates that this is not true. Using the technology according to the present invention it is possible to screen rationally for soaking solution compositions that are not derived from the original crystallization solution that was used for nucleation, growth and/or storage of the respective macromolecular crystals. The inventors have demonstrated that one key advantage is that the selected soaking solutions not derived from the original crystallization solution can be used as suitable soaking solutions for more than one biological macromolecule crystal types. In particular, some of the soaking solutions of the invention or selected with the method of the invention can be used for effectively soaking crystals from different proteins.

### DESCRIPTION

Subject-matter of the present invention is an array for soaking of biological macromolecular crystals, a method for selecting a suitable soaking solution and the composition of the soaking solution and a method of small molecule screening using the soaking solutions selected with the method of the invention.

The array of the invention comprising a first dimension of at least two individual soaking solutions (1n to xn) and a second dimension of at least two individual soaking solutions (1m to ym) wherein each of said soaking solutions is located in a separated compartment of said array, and wherein each of said soaking solution comprises an organic solvent (os), a compatible solute (cs), a crystallization solution (crs) and water, and wherein the ratio of volume compatible solute Vcs to volume organic solvent Vos is the same within a series of soaking solutions in said first dimension, or, alternatively, the molar ratio of organic solvent (os) to compatible solute (cs) Mos/Mcs is the same within a series of soaking solutions in said first dimension, and wherein the individual soaking solutions of said first dimension comprise the same organic solvent and the same compatible solute within a series of said soaking solutions in the first dimension, respectively, and wherein the ratio of the volume of water Vw and the volume of the crystallization solution Vcrs is varied over the individual soaking solutions of the series in the first dimension and wherein one of the individual soaking solutions of the series in the first dimension has a minimal or zero Vcrs and a maximum Vw and another individual soaking solution of said first dimension has a minimal or zero Vw and a maximum Vcrs and wherein the other individual soaking solutions of the series in said first dimension take values of Vcrs and Vw in between the two before-mentioned values.

In a particular embodiment of the invention 1 n to xn defines the numbers of individual soaking solution compositions in a range of between 1 (1n) to 1×106 (xn) and wherein the numbers comprise but are not limited to 1×106 or 1×105 1×104 or 768 or 384 or 96 or 48 or 24, however, the set-up of the array determines the numbers of individual soaking solutions.

In another particular embodiment of the invention 1 m to ym defines the numbers of individual soaking solution compositions in a range of between 1 (1m) to 1×106 (ym) and wherein the numbers comprise but are not limited to 1×106 or 1×105 1×104 or 768 or 384 or 96 or 48 or 24, however, the set-up of the array determines the numbers of individual soaking solutions.

Subject matter of the present invention is an array according to the present invention wherein the array comprises a second dimension (dll) of a series of individual soaking solutions for a biological macromolecular crystal wherein the compatible solute is varied over said second dimension and the ratio of volume compatible solute Vcs to volume organic solvent Vos or, alternatively, the molar ratio of organic solvent (os) to compatible solute (cs) Mos/Mcs, may be different dependent on the compatible solute used in the series of soaking solutions in said second dimension.

In one embodiment in each of said individual soaking solutions in a series the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs:Vos).

In one embodiment in each of said individual soaking solutions in a series the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10.

Subject matter of the present invention is an array according to the present invention wherein the array comprises at least x series in said first (1 n to xn) and second dimension (1m to ym) of individual soaking solutions for soaking a biological macromolecular crystal, and wherein the compatible solute or the other components of the soaking solution of said x series in said second dimension of individual soaking solutions can be varied. Variations in the dimension dll may be due to how many compatible solutes are tested in the array or variations in the dimension dll are due to how many combinations of compatible solutes and or organic solvents are tested in the array.

Subject matter of the present invention is an array according to the present invention, wherein the total volume of each of the individual soaking solutions is usually in a range of μm to ml, typically between 2.5 μl and 10 μl.

Unless the volume is expressed specifically in standard measures of volume e.g. μm, ml, “V” such as in Vos, Vcs, Vcrs and Vw always refers to the proportion/percentage of the relevant component of the solution to the total volume (VT) of the solution ie.100%. For instance Vos refers to the percentage/proportion of organic solvent (os) in the whole volume of solution e.g. Vos=10 means os is 10% of the total volume of the solution.

The molar ratio Mos/Mcs refers to the ratio of the number of moles of organic solvent (os) to the number of moles of compatible solute (cs).

The proportion of organic solvent (os) to a liquid compatible solute (cs) may be defined either through the ratio of volume compatible solute Vcs to volume organic solvent Vos, that is Vcs:Vos, or, alternatively, through the molar ratio of organic solvent (os) to compatible solute (cs) Mos/Mcs. Both definitions are considered equivalent within the invention. The proportion of organic solvent (os) to a solid compatible solute (cs) is defined through the molar ratio of organic solvent (os) to compatible solute (cs) Mos/Mcs. Mixing a liquid compatible solute (cs) with organic solvent (os) in a given molar ratio provides the same effect as mixing a solid compatible solute (cs) with organic solvent (os) in the same molar ratio.

The proportion of liquid compatible solute (cs) to organic solvent (os) may be defined as mentioned before either through the ratio of volume compatible solute Vcs to volume organic solvent Vos, that is Vcs:Vos, or, alternatively, through the concentration Ccs in mol per liter or number of moles, respectively, of compatible solute Ncs per volume organic solvent Vos, that is Ccs=Ncs:Vcs. Both definitions are considered equivalent within the invention. The proportion of a solid compatible solute (cs) to organic solvent (os) may be defined through the concentration Ccs or number of moles compatible solute Ncs per volume organic solvent Vos, respectively.

Ncs refers to the number of moles of compatible solute (cs) and Nos refers to the number of moles of organic solvent (os).

Depending on the required precision in a given array the preparation of the solution e.g. by pipetting technique may differ. If a high precision is required, low volumes in a pico- to microliter range can be pipetted by automated pipetting (i.e. a pipetting robot). However, for logical reasons volumes in a micro-milliliter range are preferably used in the array as they can be pipetted by hand (i.e. by technical assistance). Nevertheless, the volume that can be used in the array is only limited by the plate (custom made in a i.e. 3D printer or commercially available) in which the soaking is performed.

In a particular embodiment of the invention the soaking time is in a range of seconds to 24 months, preferably to 52 weeks, more preferably 72 hours. Preferably, the soaking time is from 6 hours to 24 hours.

Subject matter of the present invention is an array according to the present invention wherein the compatible solute is selected from the group comprising polyols, amino acids, methylamines and other compatible solutes.

In a particular embodiment of the invention the array comprises a compatible solute wherein the polyols are selected from a group comprising poly(oxyethylene), polyhydric alcohols, monosaccharide, disaccharide, trisaccharide, sugar alcohols, cyclitols and derivatives of former molecules. Mixtures of any of the compatible solutes can also be used.

In a further particular embodiment of the present invention the group of poly(oxyethylene) and polyhydric alcohols comprises monodisperse or polydisperse poly(oxyethylene) of molecular weights ranging between 20-2000000 g/mol and includes but is not limited to for example polyethylene glycol 400, and polyhydric alcohols comprising propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol).

In another further particular embodiment of the present invention the group of monosaccharides comprises preferably but is not limited to i.e. (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside).

In an additional particular embodiment of the present invention the group of disaccharides comprises preferably but is not limited to i.e. (p-D-Fructofuranosyl α-D-glucopyranoside (sucrose), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose).

In another particular embodiment of the present invention the group of trisaccharides comprises preferably but is not limited to i.e. (2R,3R,4S,5S,6R)-2-[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol (raffinose).

In another particular embodiment of the present invention the group of sugar alcohols comprises preferably but is not limited to i.e. (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)).

In another embodiment of the present invention the group of cyclitols comprises preferably but is not limited to i.e. (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside).

In an additional particular embodiment of the present invention the group of derivatives of former molecules comprises preferably but is not limited to i.e. mannose derivatives as fiorin ((2R)—O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose. (In all possible stereoisomers).

In another particular embodiment of the invention the array comprises a compatible solute wherein the polyols are selected from a preferred group comprising poly(oxyethylene) of molecular weights ranging between 200-20000 g/mol and includes but is not limited to poly(oxyethylene) (200 g/mol, 300 g/mol, 400 g/mol, 550 g/mol, 600 g/mol, 1000 g/mol, 1500 g/mol, 2000 g/mol, 3350 g/mol, 4000 g/mol, 5000 g/mol, 6000 g/mol, 8000 g/mol, 10000 g/mol, 20000 g/mol), plyhydric alcohols selected from the group comprising propane-1,2,3-triol (glycerol), ethane-1,2-diol (ethylene glycol), 2-Methylpentane-2,4-diol (2-Methylpentane-2,4-diol), and saccharides selected from the group comprising (3S,4R,5R)-1,3,4,5,6-Pentahydroxyhexan-2-one (fructose), (2R,3S,4R,5R)-2,3,4,5,6-Pentahydroxyhexanal (glucose), (2S,3R,4R,5R)-Hexane-1,2,3,4,5,6-hexol (sorbitol), (β-D-Fructofuranosyl α-D-glucopyranoside (sucrose), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose)), inositol derivatives (preferably di-myoinositol-1,1′-phosphate).

In a further particular embodiment of the invention the array comprises a compatible solute wherein the polyols are selected from a more preferred group comprising poly(oxyethylene) of molecular weights ranging between 400-20000 g/mol and includes but is not limited to poly(oxyethylene) (400 g/mol, 550 g/mol, 3350 g/mol, 6000 g/mol, 8000 g/mol, 10000 g/mol, 20000 g/mol), and polyhydric alcohols selected from the group comprising propane-1,2,3-triol (glycerol), ethane-1,2-diol (ethylene glycol), 2-Methylpentane-2,4-diol (2-Methylpentane-2,4-diol), and saccharides selected from the group comprising β-D-Fructofuranosyl α-D-glucopyranoside (sucrose), (2R,3S,4R,5R)-2,3,4,5,6-Pentahydroxyhexanal (glucose), (2S,3R,4R,5R)-Hexane-1,2,3,4,5,6-hexol (sorbitol), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose).

In an additional particular embodiment of the invention the array comprises a compatible solute wherein the polyols are selected from a most preferred group comprising poly(oxyethylene) of molecular weights ranging between 400-20000 g/mol and includes but is not limited to poly(oxyethylene) (400 g/mol), polyhydric alcohols selected from the group comprising propane-1,2,3-triol (glycerol), ethane-1,2-diol (ethylene glycol), 2-Methylpentane-2,4-diol (2-Methylpentane-2,4-diol), or saccharides selected from the group comprising (2R,3S,4S,5R,6R)-2-(Hydroxymethyl)-6-[(2R,3R,4S,5S,6R)-3,4,5-trihydroxy-6-(hydroxymethyl)oxan-2-yl] oxyoxane-3,4,5-triol (trehalose), (2S,3R,4R,5R)-Hexane-1,2,3,4,5,6-hexol (sorbitol), β-D-Fructofuranosyl α-D-glucopyranoside (sucrose). Mixtures of compatible solute such as poly(oxyethylene) can also be used.

According to the present invention the group of amino acids (amino-group in alpha and beta position from the carboxylate group) comprises preferably 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[[(1R)-1-carboxyethyl]amino]-5-(diaminomethylideneamino)pentanoic acid (Octopin). It shall be noted that the above mentioned amino acids selected from the preferred group can occur in all possible stereoisomers. Mixtures of any of the above compatible solutes can also be used.

According to the present invention the group of methylamines comprises preferably N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).

According to the present invention the group of other compatible solutes comprises: 1-methyl-2-pyridinecarboxylic acid (homarine), methylsulfonium solutes as 3-dimethylsulfoniopropanoate (Dimethylsulfoniopropionate), and methylsulfinylmethane as dimethyl sulfoxide, urea (and derivatives) and other compounds with traits of compatible solutes according to the definition.

Subject matter of the present invention is an array according to the present invention wherein the compatible solute is selected from the most preferred group comprising: PEG400, MPD, EG and glycerol.

According to the present invention, mixtures of single compatible solutes selected from different groups and the same group of the compatible solutes comprising polyols, amino acids, methylamines and other compatible solutes can be used in the array. Thus, the term compatible solutes according to the present invention may refer to a mixture of compatible solutes. In a preferred embodiment, cs is a mixture of PEG, preferably PEG400 and PEG3350.

In a preferred embodiment of the invention is the method of selecting the composition of a soaking solution, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules, and wherein the amino acids are selected from a group comprising 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[[(1R)-1-carboxyethyl]amino]-5-(diaminomethylideneamino)pentanoic acid (Octopin) and wherein the methylamines are selected from a group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).

In another preferred embodiment of the invention is the method of selecting the composition of a soaking solution, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules wherein the group of poly(oxyethylene) is selected from a group comprising monodisperse or polydisperse poly(oxyethylene) of molecular weights ranging between 20-2000000 g/mol and includes but is not limited to for example polyethylene glycol 400, polyethylene glycol 600 and wherein the polyhydric alcohol is selected from the group comprising propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol) and wherein the monosaccharide is selected from the group comprising (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside) and wherein the group of disaccharides is selected from the group comprising β-D-Fructofuranosyl α-D-glucopyranoside (sucrose), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose) and wherein the group of trisaccharides is selected from the group comprising (2R,3R,4S,5S,6R)-2-[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol (raffinose) and wherein the group of sugar alcohols comprises (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)) and wherein the group of cyclitols comprises (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside) and wherein the group of derivatives of former molecules comprises mannose derivatives as fiorin ((2R)-O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose.

In one embodiment of the above the described array, the array comprises a first dimension of a series of at least two individual soaking solutions for the soaking of a biological macromolecule wherein the compatible solute of the second dimension of a series of at least two individual soaking solutions differs from the one of the first dimension. In one embodiment, the described array, the array comprises of 1x to xn series in said first dimension of individual soaking solutions for the biological macromolecule wherein the compatible solute of each of the series of individual soaking solution in the first dimension differs from the one of the series of individual soaking solutions of the second dimension.

Subject matter of the present invention is an array according to the present invention, wherein the organic solvent comprises liquid carbohydrates, protic or aprotic, of low reactivity, that can serve to solve small molecules. The examples of the organic solvents include, but are not limited to: straight or branched chain monohydric aliphatic alcohols, such as methanol, ethanol, propan-1-ol, propan-2-ol, 2-methylpropan-1-ol (isobutanol), butan-1-ol (n-butanol), 2-methylpropan-2-ol (tert-butanol); di- or polyhydric aliphatic alcohols, such as 2-methyl-2,4-pentanediol hexane-2,5-diol (2,5-Hexanediol), propane-1,3-diol (1,3-propanediol), butane-2,3-diol, propane-1,2-diol (1,2-propanediol), ethane-1,2-diol (ethylen glycol); monoalkyl ethers of an aliphatic dihydric alcohols, such as monomethyl ether of ethylene glycol; dialkyl ketones, such as propan-2-one (acetone); cyclic non-aromatic ethers, such as oxolane (tetrahydrofuran), crown ethers, 1,4-dioxane (dioxane); cyclic enol-ethers, such as furan; benzyl alcohols; phenyl ethyl alcohols; (alkylated) sulfoxides, such as methylsulfinylmethane (DMSO) and its derivatives; esters derived from acetic acids, such as ethyl acetate and its derivatives; alkylated formamides, such as N,N-dimethylformamide; organic nitriles, such as acetonitrile; lactones such as oxolan-2-one (gamma-butyrolactone), 4-methyloxetan-2-one (beta-butyrolactone); polyethers, such aspolyethylene glycol (for example 400); polyetheramines, such as Jeffamine (for example 400); glycolethers, such as 2-(2-ethenoxyethoxy)ethanol (diethylene glycol); and others suitable to dissolve small molecules for protein crystal-based screenings.

In an embodiment of the invention is a method of selecting the composition of a soaking solution, wherein the organic solvent is a straight or branched chain monohydric aliphatic alcohol, dihydric aliphatic alcohol, monoalkyl ether of an aliphatic dihydric alcohol, ether of an aliphatic monohydric alcohol, cyclic non-aromatic ethers, (cyclic) enol-ethers, dialkyl ketones, benzyl alcohols, phenyl ethyl alcohols, (alkylated) sulfoxides and according phosphoderivatives, esters derived from acetic acid, (alkylated) formamides or mixtures thereof.

In one aspect, the present invention is an array according to the present invention, wherein the crystallization solution crs that is obtained from the crystallization process of said biological macromolecular crystal comprises pH and buffer materials, additives, and precipitants. According to the present invention, mixtures of single components of the crystallization solution selected from different groups and the same group of the single components of the crystallization solution comprising pH and buffer materials, additives, and precipitants can be used in the array, the soaking solution and the method of the invention

According to the present invention pH and buffer materials comprise typically acetate-, TRIS, HEPES-, cacodylate-, 1H-imidazole (imidazole), citrate-buffer and others.

According to the present invention precipitants typically comprise volatile organic solvents, salts, polymers and non-volatile organic solvents.

In a particular embodiment of the invention the preferred group of volatile organic solvents comprises typically ethanol, propan-1-o, propan-2-ol, 2-propanol, 1,4-dioxane (dioxane), propan-2-one (acetone), 2-methylpropan-1-ol (isobutanol), butan-1-ol (n-butanol), 2-methylpropan-2-ol (tert-butanol), acetonitrile, methylsulfinylmethane (dimethyl sulfoxide), oxolan-2-one (gamma-butyrolactone), 4-methyloxetan-2-one (beta-butyrolactone)).

In another particular embodiment of the invention the preferred group of salts comprises typically disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), triazanium; 2-hydroxypropane-1,2,3-tricarboxylate (ammonium citrate), sodium; acetate (sodium ethanoate), azanium; acetate (ammonium acetate), dilithium; sulfate, lithium; chloride, lithium; acetate, lithium; formate, lithium; nitrate, sodium; nitrate, sodium; chloride, potassium; chloride, sodium; formate, monophosphate from sodium and potassium, di- and polyphosphates from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium; diformate, magnesium; dichloride, magnesium; sulfate, calcium; dichloride; dihydrate, disodium; butanedioate (sodium succinate), cadmium(II) sulfate (cadmium sulfate), disodium; propanedioate (sodium malonate), magnesium; diacetate (magnesium acetate), zinc; diacetate (zinc acetate), calcium; diacetate (calcium acetate).

In a further particular embodiment of the invention the preferred group of polymers typically comprises polyethylene glycols (preferably PEG400, PEG1000, PEG3350, PEG4000, PEG6000, PEG8000, PEG20000), Jeffamine T, Jeffamine M, 2-methoxyethanol (polyethylene glycol monomethyl ester), 2-hydroxyethyl octadecanoate (polyethylene glycol monostearate), polyeneamine. The PEG can be used in a pure form or diluted e.g 50% diluted in water. In an additional particular embodiment of the invention the preferred group of non-volatile organic solvents comprises 2-methyl-2,4-pentanediol, hexane-2,5-diol (2,5-Hexanediol), propane-1,3-diol (1,3-propanediol), polyethylene glycol (for example 400), Jeffamine (for example 400).

According to the present invention the group of additives comprises physiologically or biochemically relevant small molecules, chemical protectants, solubilizing agents and detergents, compounds that reduce twinning, osmolytes, co-solvents and cosmotropes, classes of compounds that cross-link carboxyl and amino groups on the protein surface, classes of compounds that form electrostatic, covalent or hydrogen-bonding arrangements intended to stabilize the biological macromolecular crystals and classes of materials or compounds that serve to enhance nucleation and providing unique surfaces.

In a particular embodiment of the invention the preferred group of additives comprises in respect to the protein physiologically or biochemically relevant small molecules as for example 5-[(3aS,4S,6aR)-2-oxo-1,3,3a,4,6,6a-hexahydrothieno[3,4-d]imidazol-4-yl]pentanoic acid biotin, [[(2R,3S,4R,5R)-5-(6-aminopurin-9-yl)-3,4-dihydroxyoxolan-2-yl]methoxy-hydroxyphosphoryl] phosphono hydrogen phosphate (adenosine triphosphate), [(2R,3S,4R,5R)-5-(6-aminopurin-9-yl)-3,4-dihydroxyoxolan-2-yl]methyl phosphono hydrogen phosphate (adenosine diphosphate), [(2R,3S,4R,5R)-5-(6-aminopurin-9-yl)-3,4-dihydroxyoxolan-2-yl]methyl dihydrogen phosphate (adenosine monophosphate), 3-[18-(2-carboxylatoethyl)-7,12-bis(ethenyl)-3,8,13,17-tetramethylporphyrin-21,23-diid-2-yl]propanoate; iron (Fe-Protoporphyrin IX or heme b).

In another particular embodiment of the invention the preferred group of additives comprises chemical protectants for example reductants as 2-sulfanylethanol (β-mercaptoethanol or BME) and (2S,3S)-1,4-bis(sulfanyl)butane-2,3-diol (dithiothreitol or DTT), 3-[bis(2-carboxyethyl)phosphanyl]propanoic acid; hydrochloride (TCEP), heavy-metal ion scavengers as 2-[2-[bis(carboxymethyl)amino]ethyl-(carboxymethyl)amino]acetic acid (Ethylenediaminetetraacetic acid or EDTA) and 2-[2-[2-[2-[bis(carboxymethyl)amino]ethoxy]ethoxy]ethyl-(carboxymethyl)amino]acetic acid (EGTA), compounds to prevent microbial infections as sodium; azide (sodium azide), phenol, 1,1,1-trichloro-2-methylpropan-2-ol (chlorobutanol).

In a further particular embodiment of the invention the preferred group of additives comprises solubilizing agents and detergents as quaternary ammonium salts, sulfobetains, chaotropes as urea, as well as surfactants and detergent molecules as (2R,3S,4S,5R,6R)-2-(hydroxymethyl)-6-octoxyoxane-3,4,5-triol (n-octyl-beta-d-glucoside), octyl-polyoxyethylene (octyl-POE), N,N-dimethyldodecan-1-amine oxide (lauryldimethylamine oxide), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (n□dodecyl□β□D□maltoside), sodium; 1,4-bis(2-ethylhexoxy)-1,4-dioxobutane-2-sulfonate (bis(2-ethylhexyl)sulfosuccinate), dodecylazanium; chloride (dodecylammonium chloride), polyethoxylated fatty acids.

In an additional particular embodiment of the invention the preferred group of additives comprises compounds that are meant to reduce twinning as ethanol, methylsulfinylmethane (dimethyl sulfoxide or DMSO), propan-2-one (acetone), 1,4-dioxane (dioxane), butan-1-ol (butanol), 2-methylpentane-2,4-diol (2-Methyl-2,4-pentanediol or MPD).

In another particular embodiment of the invention the preferred group of additives comprises osmolytes, co-solvents and cosmotropes as (2R,3R,4S,5S,6R)-2-[(2S,3S,4S,5R)-3,4-dihydroxy-2,5-bis(hydroxymethyl)oxolan-2-yl]oxy-6-(hydroxymethyl)oxane-3,4,5-triol (sucrose), (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) and other sugars (2S)-pyrrolidine-2-carboxylic acid (proline), N,N-dimethylmethanamine oxide (trimethylamine N-oxide or TMAO), 2-aminoacetic acid (glycine), 2-(trimethylazaniumyl)acetate (betaine), 2-aminoethanesulfonic acid (taurine), 2-(methylamino)acetic acid (sarcosine) and others and their respective stereoisomers if existent.

In another particular embodiment of the invention the preferred group of additives comprises classes of compounds that cross-link carboxyl and amino groups on the surface of proteins as diamino-containing or dicarboxylic acid-containing molecules or aliphatic moieties of various length carrying charged groups.

In a further particular embodiment of the invention the preferred group of additives comprises classes of compounds that form electrostatic, covalent or hydrogen-bonding arrangements intended to stabilize crystals by intermolecular cross-linking between proteins in a crystal for example pentanedial (glutaraldehyde).

In another particular embodiment of the invention the preferred group of additives comprises classes of materials or compounds that serve to enhance nucleation and providing unique surfaces as PEG, Jeffamine, gels, gels as used in cubic lipidic phase crystallization and surfaces which promote epitaxy and heterogeneous nucleation, commercially available nucleants like Crystallophore No. 1, Naomi's nucleant, JBS Magical Triangle, Anderson-Evans polyoxotungstate (Na6[TeW6024]×22 H2O) or components of buffer solutions that influence the macromolecule's solubility and aggregation behavior and as a consequence their nzcleation and growth behavior as 2-aminoacetic acid (glycine), 3-[4-(3-sulfopropyl)piperazin-1-yl]propane-1-sulfonic acid (PIPPS), 2-[bis(2-hydroxyethyl)amino]-2-(hydroxymethyl)propane-1,3-diol (Bis-Tris), 2-morpholin-4-ylethanesulfonic acid (MES), 2-[(2-amino-2-oxoethyl)-(carboxymethyl)amino]acetic acid (ADA), 2-[3-[[1,3-dihydroxy-2-(hydroxymethyl)propan-2-yl]amino]propylamino]-2-(hydroxymethyl)propane-1,3-diol (Bis-Tris Propane), 3-morpholin-4-ylpropane-1-sulfonic acid (MOPS), 2-[4-(2-hydroxyethyl)piperazin-1-yl]ethanesulfonic acid (HEPES), 2-amino-2-(hydroxymethyl)propane-1,3-diol (TRIS), 3-[4-(2-hydroxyethyl)piperazin-1-yl]propane-1-sulfonic acid (EPPS), 1H-imidazole (imidazole), 2-[bis(2-hydroxyethyl)amino]acetic acid (bicine), 2-(cyclohexylamino)ethanesulfonic acid (CHES), 3-(cyclohexylamino)propane-1-sulfonic acid (CAPS), (2R,3S,4S,5R)-2-(hydroxymethyl)-6-octoxyoxane-3,4,5-triol (octyl glucopyranoside), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (dodecyl maltoside), (2S,3S)-1,4-bis(sulfanyl)butane-2,3-diol (DTT), 3-[bis(2-carboxyethyl)phosphanyl]propanoic acid (TCEP).

Class of cryoprotectants are polyethylene glycols like PEG200, PEG 400, P600 etc., ethane-1,2-diol (ethylene glycol), 2-methylpentane-2,4-diol (2-Methyl-2,4-pentanediol or MPD), N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), propane-1,2,3-triol (glycerol), (2S,4R)-pentane-1,2,3,4,5-pentol xylitol or ribitol), (2R,3R,4S,5S,6R)-2-[(2S,3S,4S,5R)-3,4-dihydroxy-2,5-bis(hydroxymethyl)oxolan-2-yl]oxy-6-(hydroxymethyl)oxane-3,4,5-triol (sucrose), (3R,4S,5S,6R)-6-(hydroxymethyl)oxane-2,3,4,5-tetrol (glucose), (2S,3R)-butane-1,2,3,4-tetrol (erythritol), propan-1-ol (propanol), propan-2-ol (isopropanol), ethanol, 2-ethoxyethanol (ethylene glycol monoethyl ether), (2R,3R)-butane-2,3-diol; (1R,2R)-cyclohexane-1,2-diamine ((2R,3R)butane 2,3 diol), (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (threhalose), 2-aminoethanesulfonic acid (taurine), (6S)-2-methyl-1,4,5,6-tetrahydropyrimidine-6-carboxylic acid (ectoine), (2S)-pyrrolidine-2-carboxylic acid (proline) and others as well as derivatives and stereoisomers.

In the present application the crystallization solution (crs) refers both to 1) the crystallization solution crs as described above that is obtained from the crystallization process of the biological macromolecular crystal and 2) to a freshly prepared solution of chloride salts such as NaCl, MgCl₂ and KCl.

In the prior art, it is believed that macromolecular crystals from every individual protein species require individual soaking solution compositions that are based on their original crystallization condition. One aspect of the present invention actually demonstrates that this is not true. Using the technology according to the present invention it is possible to screen rationally for soaking solution compositions that are not derived from the original crystallization solution that was used for nucleation, growth and/or storage of the respective macromolecular crystals. Instead, and counterintuitively, salt solutions such as NaCl or KCl or any other salt that is generally applicable in crystallization experiments or used for buffer compositions can maintain the integrity of macromolecular crystals. The method according to the present invention allows for identifying a suitable combination of types of salt, compatible solutes and organic solvents as well as their suitable concentrations. This demonstrates that soaking solutions can be rendered independent from the original crystallization conditions. Interestingly and counterintuitively, very different crystal species that were grown under very different crystallization conditions can be stabilized and soaked in some of the soaking solutions identified by the application of an embodiment of this invention.

The freshly prepared crs consists in a salt solution that is not the original crs but nevertheless is equivalent in terms of osmotic net pressure, solubility of the biological macromolecule, permittivity, and structural conformation, maintains some degree of ionic strength or buffer capacity and preserves the crystals integrity. Typically, the freshly prepared crs is referred as an equivalent salt solution different from original crs used for nucleation, growth and/or storage of the macromolecular crystal, and wherein the salt of the solution is any salt that can be used for crystallization of biological macromolecules.

Another embodiment of the invention is a method of selecting the composition of a soaking solution, wherein an equivalent aqueous salt solution different from the crystallization solution is used, and wherein the salt selected from the group comprising disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), triazanium; 2-hydroxypropane-1,2,3-tricarboxylate (ammonium citrate), sodium; acetate (sodium ethanoate), azanium; acetate (ammonium acetate), dilithium; sulfate, lithium; chloride, lithium; acetate, lithium; formate, lithium; nitrate, sodium; nitrate, sodium; chloride, potassium; chloride, sodium; formate, monophosphate from sodium and potassium, di- and polyphosphates from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate)), magnesium; diformate, magnesium; dichloride, magnesium; sulfate, calcium; dichloride; dihydrate, disodium; butanedioate (sodium succinate), cadmium(II) sulfate (cadmium sulfate), disodium; propanedioate (sodium malonate), magnesium; diacetate (magnesium acetate), zinc; diacetate (zinc acetate), calcium; diacetate (calcium acetate) and wherein the salt for the equivalent solution is preferably selected from the group comprising lithium; acetate, lithium; chloride, lithium; formate, lithium; nitrate, dilithium; sulfate, sodium; chloride, potassium; chloride, magnesium; dichloride, diazanium; sulfate (ammonium sulfate), sodium; acetate (sodium ethanoate), sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium; diformate, sodium; nitrate.

In another embodiment of the invention, the equivalent aqueous solution is suitable for the storage of a macromolecular crystal and to replace the original crs while contributing to the maintenance of crystal integrity preserving parameters, especially in terms of osmotic net pressure, permittivity, solubility of the according biological macromolecule, and effect on the biological macromolecule's structural conformation and is not suitable not intended to facilitate protein nucleation and crystal growth or is used for storage of a macromolecular crystal but is intended to replace the original crs while contributing to the maintenance of crystal integrity preserving parameters, especially in terms of osmotic net pressure, permittivity, solubility of the according biological macromolecule, and effect on the biological macromolecule's structural conformation.

In another particular embodiment of the invention the preferred group of salts for the equivalent salt solution comprises typically disodium disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), Ammonium 2-hydroxypropane-1,2,3-tricarboxylate (ammonium citrate), sodium ethanoate (sodium acetate), ammonium ethanoate (ammonium acetate), lithium sulfate, lithium chloride, sodium chloride, potassium chloride, sodium methanoate (sodium formate), monophosphate from sodium and potassium, di- and polyphospahtes from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium diformate, magnesium dichloride, magnesium sulfate, calcium chloride, disodium butanedioate (sodium succinate), cadmium(II) sulfate (cadmium sulfate), Disodium propanedioate (sodium malonate), magnesium; diacetate (magnesium acetate), zinc; diacetate (zinc acetate), calcium; diacetate (calcium acetate).

In one embodiment, the salt solution (crs) is an NaCl, MgCl₂ or KCl solution or contains NaCl, MgCl₂ or KCl. Preferably, the concentration of NaCl, MgCl₂ or KCl is between 0.1M and 6M. Preferably, the NaCl, MgCl₂ or KCl concentration is 1M. Advantageously, the freshly prepared crs renders the process independent from the original crs. In a preferred embodiment, the same selected soaking solution(s) is(are) suitably used for soaking crystals of different biological macromolecules, in particular different proteins.

A further embodiment of the invention is a method of selecting the composition of a soaking solution, wherein the salt is sodium; chloride, potassium; chloride, magnesium; diacetate (magnesium acetate) and dilithium; sulfate.

Preferably, when a salt solution of freshly prepared crs is used in the soaking solution, the cs is a mixture of PEG, more preferably PEG400 and PEG3350.

In a preferred embodiment, the crs of the soaking solution is a solution of NaCl, MgCl₂ or KCl and/or the cs is 3350, PEG 400 or a mix of PEG400 and 3350.

In another embodiment, the crs does not contain any Cl salts and in particular any NaCl.

An embodiment of the present invention is a method of selecting the composition of a soaking solution, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the pH and buffer materials are selected from a group comprising acetate-, TRIS, HEPES-, cacodylate-, 1H-imidazole (imidazole) and citrate-buffer and wherein the precipitant is selected from a group comprising volatile organic solvents, salts, polymers or non-volatile organic solvents, and wherein the additives are selected from a group comprising physiologically or biochemically relevant small molecules, chemical protectants, solubilizing agents and detergents, compounds that reduce twinning, osmolytes, co-solvents and cosmotropes, classes of compounds that cross-link carboxyl and amino groups on the protein surface, classes of compounds that form electrostatic, covalent or hydrogen-bonding arrangements intended to stabilize the biological macromolecular crystals and classes of materials or compounds that serve to enhance nucleation and providing unique surfaces.

A preferred embodiment of the invention is a method of selecting the composition of a soaking solution, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the precipitant is selected from a group comprising volatile organic solvents, salts, polymers or non-volatile organic solvents and wherein the volatile organic solvents is selected from a group comprising ethanol, propan-1-o, propan-2-ol, 2-propanol, 1,4-dioxane (dioxane), propan-2-one (acetone), 2-methylpropan-1-ol (isobutanol), butan-1-ol (n-butanol), 2-methylpropan-2-ol (tert-butanol), acetonitrile, methylsulfinylmethane (dimethyl sulfoxide), oxolan-2-one (gamma-butyrolactone), 4-methyloxetan-2-one (beta-butyrolactone)) and wherein the salt for the equivalent salt solution is selected from the group comprising disodium disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), Ammonium 2-hydroxypropane-1,2,3-tricarboxylate (ammonium citrate), sodium ethanoate (sodium acetate), ammonium ethanoate (ammonium acetate), lithium sulfate, lithium chloride, sodium chloride, potassium chloride, sodium methanoate (sodium formate), monophosphate from sodium and potassium, di- and polyphospahtes from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium diformate, magnesium dichloride, magnesium sulfate, calcium chloride, disodium butanedioate (sodium succinate), cadmium(II) sulfate (cadmium sulfate), Disodium propanedioate (sodium malonate), magnesium; diacetate (magnesium acetate), zinc; diacetate (zinc acetate), calcium; diacetate (calcium acetate) and wherein the polymers is selected from a group comprising polyethylene glycols (preferably PEG400, PEG1000, PEG3350, PEG4000, PEG6000, PEG8000, PEG20000), Jeffamine T, Jeffamine M, 2-methoxyethanol (polyethylene glycol monomethyl ester), 2-hydroxyethyl octadecanoate (polyethylene glycol monostearate) and polyeneamine and wherein the non-volatile organic solvents is selected from a group comprising 2-methyl-2,4-pentanediol, hexane-2,5-diol (2,5-Hexanediol), propane-1,3-diol (1,3-propanediol), polyethylene glycol (for example 400), Jeffamine (for example 400).

Another preferred embodiment of the invention is a method of selecting the composition of a soaking solution, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the group of additives is selected from a group comprising physiologically or biochemically relevant small molecules, chemical protectants, solubilizing agents and detergents, compounds that reduce twinning, osmolytes, co-solvents and cosmotropes, classes of compounds that cross-link carboxyl and amino groups on the protein surface, classes of compounds that form electrostatic, covalent or hydrogen-bonding arrangements intended to stabilize the biological macromolecular crystals and classes of materials or compounds that serve to enhance nucleation and providing unique surfaces wherein the protein physiologically or biochemically relevant small molecule is selected from a group comprising 5-[(3aS,4S,6aR)-2-oxo-1,3,3a,4,6,6a-hexahydrothieno[3,4-d]imidazol-4-yl]pentanoic acid biotin, [[(2R,3S,4R,5R)-5-(6-aminopurin-9-yl)-3,4-dihydroxyoxolan-2-yl]methoxy-hydroxyphosphoryl] phosphono hydrogen phosphate (adenosine triphosphate), [(2R,3S,4R,5R)-5-(6-aminopurin-9-yl)-3,4-dihydroxyoxolan-2-yl]methyl phosphono hydrogen phosphate (adenosine diphosphate), [(2R,3S,4R,5R)-5-(6-aminopurin-9-yl)-3,4-dihydroxyoxolan-2-yl]methyl dihydrogen phosphate (adenosine monophosphate), 3-[18-(2-carboxylatoethyl)-7,12-bis(ethenyl)-3,8,13,17-tetramethylporphyrin-21,23-diid-2-yl]propanoate; iron (Fe-Protoporphyrin IX or heme b) and wherein the chemical protectants are selected from a group comprising 2-sulfanylethanol (β-mercaptoethanol or BME) and (2S,3S)-1,4-bis(sulfanyl)butane-2,3-diol (dithiothreitol or DTT), 3-[bis(2-carboxyethyl)phosphanyl]propanoic acid; hydrochloride (TCEP), heavy-metal ion scavengers as 2-[2-[bis(carboxymethyl)amino]ethyl-(carboxymethyl)amino]acetic acid (Ethylenediaminetetraacetic acid or EDTA) and 2-[2-[2-[2-[bis(carboxymethyl)amino]ethoxy]ethoxy]ethyl-(carboxymethyl)amino]acetic acid (EGTA), compounds to prevent microbial infections as sodium; azide (sodium azide), phenol, 1,1,1-trichloro-2-methylpropan-2-ol (chlorobutanol) and wherein the solubilizing agents and detergents are selected from a group comprising quaternary ammonium salts, sulfobetains, chaotropes as urea, as well as surfactants and detergent molecules as (2R,3S,4S,5R,6R)-2-(hydroxymethyl)-6-octoxyoxane-3,4,5-triol (n-octyl-beta-d-glucoside), octyl-polyoxyethylene (octyl-POE), N,N-dimethyldodecan-1-amine oxide (lauryldimethylamine oxide), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (n-dodecyl-β-D-maltoside), sodium; 1,4-bis(2-ethylhexoxy)-1,4-dioxobutane-2-sulfonate (bis(2-ethylhexyl)sulfosuccinate), dodecylazanium; chloride (dodecylammonium chloride), polyethoxylated fatty acids and wherein the compounds that are meant to reduce twinning are selected from the group comprising ethanol, methylsulfinylmethane (dimethyl sulfoxide or DMSO), propan-2-one (acetone), 1,4-dioxane (dioxane), butan-1-ol (butanol), 2-methylpentane-2,4-diol (2-Methyl-2,4-pentanediol or MPD) and wherein the osmolyte, co-solvent and cosmotrope is selected from the group comprising (2R,3R,4S,5S,6R)-2-[(2S,3S,4S,5R)-3,4-dihydroxy-2,5-bis(hydroxymethyl)oxolan-2-yl]oxy-6-(hydroxymethyl)oxane-3,4,5-triol (sucrose), (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) and other sugars (2S)-pyrrolidine-2-carboxylic acid (proline), N,N-dimethylmethanamine oxide (trimethylamine N-oxide or TMAO), 2-aminoacetic acid (glycine), 2-(trimethylazaniumyl)acetate (betaine), 2-aminoethanesulfonic acid (taurine), 2-(methylamino)acetic acid (sarcosine) and wherein the class of compounds that cross-link carboxyl and amino groups on the surface of proteins is selected from the group comprising diamino-containing or dicarboxylic acid-containing molecules or aliphatic moieties of various length carrying charged groups and wherein the class of compounds that form electrostatic, covalent or hydrogen-bonding arrangements intended to stabilize crystals by intermolecular cross-linking between proteins in a crystal is pentanedial (glutaraldehyde) and wherein the class of materials or compounds that serve to enhance nucleation and providing unique surfaces is selected from the group comprising PEG, Jeffamine, gels, gels as used in cubic lipidic phase crystallization and surfaces which promote epitaxy and heterogeneous nucleation, commercially available nucleants like Crystallophore No. 1, Naomi's nucleant, JBS Magical Triangle, Anderson-Evans polyoxotungstate (Na6[TeW6024]×22 H2O) or components of buffer solutions that influence the macromolecule's solubility and aggregation behavior and as a consequence their nucleation and growth behavior as 2-aminoacetic acid (glycine), 3-[4-(3-sulfopropyl)piperazin-1-yl]propane-1-sulfonic acid (PIPPS), 2-[bis(2-hydroxyethyl)amino]-2-(hydroxymethyl)propane-1,3-diol (Bis-Tris), 2-morpholin-4-ylethanesulfonic acid (MES), 2-[(2-amino-2-oxoethyl)-(carboxymethyl)amino]acetic acid (ADA), 2-[3-[[1,3-dihydroxy-2-(hydroxymethyl)propan-2-yl]amino]propylamino]-2-(hydroxymethyl)propane-1,3-diol (Bis-Tris Propane), 3-morpholin-4-ylpropane-1-sulfonic acid (MOPS), 2-[4-(2-hydroxyethyl)piperazin-1-yl]ethanesulfonic acid (HEPES), 2-amino-2-(hydroxymethyl)propane-1,3-diol (TRIS), 3-[4-(2-hydroxyethyl)piperazin-1-yl]propane-1-sulfonic acid (EPPS), 1H-imidazole (imidazole), 2-[bis(2-hydroxyethyl)amino]acetic acid (bicine), 2-(cyclohexylamino)ethanesulfonic acid (CHES), 3-(cyclohexylamino)propane-1-sulfonic acid (CAPS), (2R,3S,4S,5R)-2-(hydroxymethyl)-6-octoxyoxane-3,4,5-triol (octyl glucopyranoside), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (dodecyl maltoside), (2S,3S)-1,4-bis(sulfanyl)butane-2,3-diol (DTT), 3-[bis(2-carboxyethyl)phosphanyl]propanoic acid (TCEP).

Another preferred embodiment of the invention is a method of selecting the composition of a soaking solution, wherein soaking solution comprises a crystallization solution (crs) or an equivalent aqueous solution thereto and an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably, said compatible solute (cs) is a poly(oxyethylene) and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the poly(oxyethylene)s PEG200, PEG400, PEG600 or a mixture thereof and said organic solvent (os) is selected from the group comprising dimethyl sulfoxide, 1,4-dioxane, dimethylformamide, methanol, ethanol, or a mixture thereof and
wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous solution Vcrs to the combined volumes of organic solvent Vos and compatible solute Vcs is 1:1000000 to 1000000:1, preferably between 99:1 to 1:99 and most preferably between 85:15 to 1:1 (Vcrs:(Vos;Vcs)).

A further preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is a polyol or a mixture thereof, and said organic solvent is a (alkylated) sulfoxide, cyclic non-aromatic ether, straight or branched chain monohydric aliphatic alcohol, alkylated formamide, or a mixture thereof,
more preferably, said compatible solute (cs) is a polyhydric alcohol or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the polyhydric alcohols propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol) or a mixture thereof, and said organic solvent (os) is selected from the group comprising dimethyl sulfoxide, 1,4-dioxane, dimethylformamide, methanol, ethanol, or a mixture thereof and
wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous solution Vcrs to the combined volumes of organic solvent Vos and compatible solute Vcs is 1:1000000 to 1000000:1, preferably between 99:1 to 1:99 and most preferably between 85:15 to 1:1 (Vcrs:(Vos; Vcs)).

Another preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably said compatible solute (cs) is a mono-, di-, tri, oligo- or polysaccharid or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute cs is selected from the group comprising the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from the group comprising dimethyl sulfoxide, 1,4-dioxane, dimethylformamide, methanol, ethanol, or a mixture thereof and
wherein the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10,
and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos;Vcs)).

A further preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is a L- or D amino acid, or a mixture thereof, and the organic solvent (os) is liquid carbohydrate protic or aprotic, of low reactivity, or a mixture thereof, that can serve to solve small molecules,
more preferably said compatible solute (cs) is a L-amino acid, or a mixture thereof, and said organic solvent is a (alkylated) sulfoxide, cyclic non-aromatic ether, straight or branched chain monohydric aliphatic alcohol, alkylated formamide, or a mixture thereof,
most preferably, said compatible solute (cs) is selected from the group comprising the L-amino acids (2S)-pyrrolidine-2-carboxylic acid (proline), 2-aminoacetic acid (glycine), (6S)-2-methyl-1,4,5,6-tetrahydropyrimidine-6-carboxylic acid (ectoine) or (5S,6S)-5-hydroxy-2-methyl-1,4,5,6-tetrahydropyrimidine-6-carboxylic acid (hydroxyectoine), or a mixture thereof, and said organic solvent (os) is selected from the group comprising dimethyl sulfoxide, 1,4-dioxane, dimethylformamide, methanol, ethanol, or a mixture thereof and
wherein the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10,
and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos;Vcs)).

Another preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is a methylamine or a mixture thereof and said organic solvent (os) is liquid carbohydrate protic or aprotic, of low reactivity, or a mixture thereof that can serve to solve small molecules,
more preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
most preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from the group comprising dimethyl sulfoxide, 1,4-dioxane, dimethylformamide, methanol, ethanol, or a mixture thereof and
wherein the molar ratio of compatible solute to organic solvent is the same and the molar ratio is 1:10000 to 10000:1, preferably between 100:1 to 1:100 and more preferably between 10:1 to 1:10,
and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos;Vcs)).

A further preferred embodiment of the invention is a method of selecting the composition of a soaking solution according to any of claims 1 to 7 and 9 wherein said soaking solution comprises a crystallization solution (crs) or an equivalent aqueous solution thereto and an organic solvent (os) and a compatible solute (cs),
wherein said compatible solute (cs) is PEG400 and said organic solvent (os) is dimethyl sulfoxide,
wherein the ratio of volume Vcs PEG400 to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous solution Vcrs to the combined volumes of the organic solvent (os) dimethyl sulfoxide and the compatible solute (cs) PEG400 is 1:1000000 to 1000000:1, preferably between 99:1 to 1:99 and most preferably between 85:15 to 1:1 (Vcrs:(Vos:Vcs)).
Another preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is ethane-1,2-diol (ethylene glycol) and said organic solvent (os) is dimethyl sulfoxide,
wherein the ratio of volume Vcs ethane-1,2-diol (ethylene glycol) to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous solution Vcrs to the combined volumes of the organic solvent (os) dimethyl sulfoxide and the compatible solute (cs) ethane-1,2-diol (ethylene glycol) is 1:1000000 to 1000000:1, preferably between 99:1 to 1:99 and most preferably between 85:15 to 1:1 (Vcrs:(Vos:Vcs)).

A further preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is propane-1,2,3-triol (glycerol) and said organic solvent (os) is dimethyl sulfoxide,
wherein the ratio of volume Vcs propane-1,2,3-triol (glycerol) to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous solution Vcrs to the combined volumes of the organic solvent (os) dimethyl sulfoxide and the compatible solute (cs) propane-1,2,3-triol (glycerol) is 1:1000000 to 1000000:1, preferably between 99:1 to 1:99 and most preferably between 85:15 to 1:1 (Vcrs:(Vos:Vcs)).
Another preferred embodiment of the invention is a method of selecting the composition of a soaking solution, wherein said soaking solution comprises a crystallization solution (crs) or an equivalent aqueous solution thereto and an organic solvent (os) and a compatible solute (cs),
wherein said compatible solute (cs) is 2-methylpentane-2,4-diol (MPD) and said organic solvent (os) is dimethyl sulfoxide,
wherein the ratio of volume Vcs 2-methylpentane-2,4-diol (MPD) to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous solution Vcrs to the combined volumes of the organic solvent (os) dimethyl sulfoxide and the compatible solute (cs) 2-methylpentane-2,4-diol (MPD) is 1:1000000 to 1000000:1, preferably between 99:1 to 1:99 and most preferably between 85:15 to 1:1 (Vcrs:(Vos:Vcs)).

According to the present invention, mixtures of single components of the different constituents of the soaking solution selected from different groups and the same group of the respective single components of the soaking solution comprising the organic solvent, a compatible solute, the crystallization solution and water can be used in the array.

In a particular embodiment of the present invention the array according to the present invention comprises each dimension of individual soaking solutions is physically arranged in dimension dl of said array. The dimension dl of individual soaking solutions may or may not be arranged physically in a row of an array. It could be also arranged differently as long as the position of each individual soaking solution may be determined bijective, i.e. in a one-to-one correspondence.

Subject matter of the present invention is the use of an array according to the present invention for testing the integrity of a biological macromolecular crystal or the use of an array according to the present invention for testing a biological macromolecular crystal regarding tolerance towards a) changes in the original growth environment by the introduction of organic solvents, compatible solutes (especially typical cryoprotectants) and dilution of original crystallization buffer, b) stress caused by i.e. changes in the pH value and/or changes in osmotic pressure in the growth environment of the crystal by introduction of i.e. organic solvents, compatible solutes, crs-dilutions and/or small molecules or by other individual properties of the used substances (organic solvents, small molecules, etc. added to conditions evaluated in a), c) or the use of an array according to the present invention for a small molecule screening process of a biological macromolecular crystal or d) the use of an array according to the present invention for identifying specific soaking solution compositions of said array that is suitable for a small molecule screening process of said biological macromolecular crystal or e) the use of an array according to the present invention for identifying specific soaking solution compositions of said array that is suitable for reducing disorder in disordered regions of the biological macromolecule.

In another aspect, the invention consists in a method of selecting a soaking solution suitable for soaking the crystal form of a biological macromolecule wherein the solution comprises an organic solvent (os), a compatible solute (cs), and a crystallization solution (crs) and/or water (w), comprising the steps of
1) preparing the soaking solution by mixing os, cs, crs and w
2) transferring at least one crystal of the biological macromolecular crystal into the solution prepared in step 1) or transferring the solution prepared in step 1) into a compartment comprising at least one crystal of the biological macromolecular crystal
3) controlling the crystal
4) selecting the soaking solution which is suitable for soaking the crystal.

In another aspect of the invention, the method of the invention is a method for selecting a soaking solution for soaking the crystal form of a biological macromolecule wherein the soaking solution comprises an organic solvent (os), a compatible solute (cs), and a crystallization solution (crs) and/or water (w); comprising the steps of
1) preparing a first (or benchmark) series of 1 n to xn individual soaking solutions by mixing os, cs, crs and w wherein:
a) x is at least 2
b) each soaking solutions of the first series comprises the same organic solvent (os), the same compatible solute (cs), and the same crystallization solution (crs), and
c) Vos and Vcs are the same in each solution, or, alternatively, Mos/Mcs is the same in each solution, and
d) Vcrs and Vw are changed in each solution, and
   in the solution 1n, Vcrs is the minimum Vcrs (Vcrsmin) and the Vw is the maximum Vw (Vwmax), and
   in the solution xn, Vw is the minimum Vw (Vwmin) and the Vcrs is the maximum Vcrs(Vcrsmax), and
   when x>2, in any additional solutions between 1n and xn, the Vcrs is varied between Vcrsmin and Vcrsmax and, inversely, Vw is varied between Vwmin and Vwmax
2) transferring at least one crystal of the biological macromolecular crystal per compartment into x compartments, each compartment comprising one of the solutions prepared in step 1); or transferring each solution prepared in step 1) into x compartments, each compartment comprising at least one crystal of the biological macromolecular crystal from its crystallization solution.
3) controlling the crystal in each compartment
4) selecting the soaking solution(s) which is/are suitable for soaking the crystal.

In a preferred embodiment, the soaking solutions of the first series are distributed in each compartment to form a first dimension. Typically, the first dimension is a line or a column of wells in a screening array.

In a further embodiment, the method for selecting the composition of a soaking solution of further comprising the step of
1) preparing m series of 1n to yn individual soaking solutions by mixing os, cs, crs and w wherein m is at least 1 and wherein in each of the m series
a) y is at least 2
b) os and crs are the same as in the first (or benchmark) series, and
c) Vos and Vcs are the same as in the first series, or, alternatively, Mos/Mcs is the same in the first series,
d) the cs is changed and is different from the cs of the first series, and
wherein in each individual solutions of each of the m series
e) Vos and Vcs are conserved, or, alternatively, Mos/Mcs is conserved, and
f) Vcrs and Vw are changed in each solution, and
   in the solution 1n, Vcrs is the minimum Vcrs (Vcrsmin) and the Vw is the maximum Vw (Vwmax), and
   in the solution yn, Vw is the minimum Vw (Vwmin) and the Vcrs is the maximum Vcrs(Vcrsmax), and
   when y>2, in any additional solutions between 1n and yn, the Vcrs is varied between Vcrsmin and Vcrsmax and, inversely, Vw is varied between Vwmin and Vwmax
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

In a most preferred embodiment, the method of selecting a soaking solution for soaking the crystal form of a biological macromolecule wherein the soaking solution comprises an organic solvent (os), a compatible solute (cs), and a crystallization solution (crs) and/or water (w); comprising the steps of
1) preparing a first series of 1 n to xn individual soaking solutions by mixing os, cs, crs and w wherein
a) x is at least 2
b) each soaking solutions of the first series comprises the same organic solvent (os), the same compatible solute (cs), and the same crystallization solution (crs), and
c) Vos and Vcs are the same in each solution, or, alternatively, Mos/Mcs is the same in each solution, and
d) Vcrs and Vw are changed in each solution, and
   in the solution 1n, V_{crs} is the minimum V_{crs} (V_{crs} min) and the V_{w} is the maximum V_{w} (V_{w} max), and
   in the solution xn, V_{w} is the minimum V_{w} (V_{w} min) and the V_{crs} is the maximum V_{crs}(V_{crs} max), and
   when x>2, in any additional solutions between 1 n and xn, the V_{crs} is varied between V_{crs} min and V_{crs} max and, inversely, Vw is varied between V_{w} min and V_{w} max
2) transferring at least one crystal of the biological macromolecular crystal per compartment into x compartments, each compartment comprising one of the solutions prepared in step 1); or transferring each solution prepared in step 1) into x compartments, each compartment comprising at least one crystal of the biological macromolecular crystal from its crystallization solution.
3) controlling the crystal in each compartment
4) selecting the soaking solution(s) which is/are suitable for soaking the crystal;
wherein the compatible solute is selected from the group comprising polyols, amino acids, methylamines, and mixtures thereof, and
wherein the organic solvent is selected from the group comprising liquid carbohydrates, protic or aprotic, of low reactivity, that can serve to solve small molecules and, mixtures thereof, and wherein the crystallization solution crs is obtained from the crystallization process of said crystal or is equivalent thereto in terms of osmotic net pressure, permittivity, solubility of the according biological macromolecule, and effect on its structural conformation, comprising pH and buffer materials, additives, and precipitants.

In a most preferred embodiment, the Vcrsmin, Vcrsmax, Vwmin and/or Vwmax are the same in each of the m series and/or the same as in the first (or benchmark) series.

In another embodiment, each of the m series, the number of individual soaking solutions y is the same as the number of individual soaking solutions x of the first series; and/or the Vcrs is incrementally varied between Vcrsmin and Vcrsmax and, inversely, Vw is incrementally varied between Vwmin and Vwmax in any additional solutions between 1n and xn and/or in any additional solutions between 1 n and yn

In a preferred embodiment, in any additional solutions between 1n and yn, the increment of Vcrs between Vcrsmin and Vcrsmax and of Vw between Vwmin and Vwmax is the same in the first series and in each of the m series.

In a preferred embodiment, the solutions of the m series are distributed in each compartment to form a second dimension. Typically, if the first dimension was represented by a line of wells (e.g. with the solution of the first series) in a standard screening or crystallization array, then the other lines of wells (e.g. containing the solutions of the m series) will generate columns of wells which will be the second dimension. Inversely, if the first dimension was represented by a column of wells (e.g. with the solution of the first series) in a standard screening or crystallization array, then the other columns of wells (e.g. containing the solutions of the m series) will generate lines of wells which will be the second dimension.

In another embodiment, the crs is a salt solution which is a freshly prepared solution ie. that is not extracted from the crystallization process and the solution that was used to grow crystals. In this embodiment, the freshly prepared crs typically consists in a salt solution that is not the original crs but nevertheless is equivalent and maintains some degree of ionic strength or buffer capacity and preserves the crystals integrity. In a preferred embodiment, the crs is a salt solution different from original crystallization solution used for nucleation, growth and/or storage of the macromolecular crystal, and wherein the salt of the crs is any salt that can be used for crystallization of biological macromolecules.

Typically, the freshly prepared crs is referred as an equivalent salt solution and preferably a solution of Cl, in particular NaCl, MgCl₂ or KCl. In a most preferred embodiment, when a salt solution of freshly prepared crs is used in the soaking solution, the cs is a mixture of PEG, more preferably PEG400 and PEG3350. In a different embodiment, the crs is a salt solution different from original crystallization solution used for nucleation, growth and/or storage of the macromolecular crystal, and wherein the salt of the crs is not a chloride (CI) salt, in particular not NaCl.

In another embodiment, the cs is selected from PEG400, MPD, EG and/or glycerol. The cs is preferably a mixture of PEG, preferably PEG400 and PEG3350 or a mixture of MPD and PEG, in particular MPD and PEG3350.

In a preferred embodiment, the os is DMSO.

A preferred V_{crs} min is 0%.

A preferred V_{w} min is 0%.

A preferred volume of os, Vₒₛ is more than 0.5%, preferably at least 10%, or at least 15% or at least 20% more preferably between 5% to 25%.

A preferred volume of cs is preferably at least 5%, more preferably 10%, or at least 20%, at least 30, at least 40%, at least 50% and more preferably between 5% and 80%, most preferred 10% to 60% In a preferred embodiment, the soaking solution consists crs, w, os and/or cs. In an embodiment, the percentage of the total volume (VT) of each solution of the first series is VT=Vcrs+Vw+Vos+Vcs=100%. In this preferred embodiment, os contains small molecules or not.

“N” such as in Nos, Ncs, Ncrs and Nw always refers to the number of moles of the relevant component. NT refers to the total number of moles of the components in the solution, ie. NT=Ncrs+Nw+Nos+Ncs.

In a preferred embodiment, the ratio of the number of moles of compatible solute to the total number of moles of the components (Ncs/NT) is at least 0.05, more preferably at least 0.1, or at least 0.2, or at least 0.3, or at least 0.4, or at least 0.5 and more preferably between 0.05 and 0.8, most preferred between 0.1 and 0.6.

Generally, and in the most preferred embodiment, the soaking solution is considered suitable for soaking the crystal when the quality of the crystal is sufficient to allow further uses and/or analysis on the crystal. Examples of such uses and/or analysis are electronic microscopy, neutron diffraction, small molecule screening, validation of hits from other screenings such as high-throughput-screenings (HTS) or biophysical screenings etc., biotechnological applications as crystal usage in chromatography, biosensors or material science (hybrid materials). In a most preferred embodiment, a soaking solution is selected if it is suitable for a small molecule screening process.

In a particular embodiment of the present invention the specific soaking solution is controlled and subsequently selected for being suitable for soaking the crystal preferably by the use of in particular X-Ray and/or by visual control according to defined standard criteria commonly used by the expert in the relevant art of crystallography and in particular in X-Ray diffraction: highest resolution which should be less than 2.8 Å. preferably less than 2.5 A, low mosaicity, typically lower than 0.8°, no or minimal ice rings, high-quality electron densities as judged by an experienced crystallographer; and for visual control: no or minimal fractures, cracks, no or minimal dissolution, sharp crystal edges, colored in polarized light etc.) giving rise to signals in a subsequent analysis. In a further additional particular embodiment of the present invention, the step of controlling the crystal and selecting the suitable solutions is performed according to defined quality criteria such as resolution, mosaicity, electron density, presence of diffraction artifacts from, for example, unwanted ice crystals or salt crystals or other crystals grown from components of the ingredients of the soaking solution composition and crystal twinning).

In the present invention, the step of controlling the crystal can be performed by any of the method available to the skilled person to evaluate the presence and/or aspect of the crystal and suitability for further analysis such as X-ray-diffraction analysis. In a preferred embodiment, crystals from different biological macromolecules, preferably proteins, are tested in the same solution(s). In particular, the step of controlling the crystal refers to assess the resistance of one or more types (i.e. from different biological macromolecule) of biological macromolecular crystals in a particular soaking solution in terms of their presence and/or to measure the impact of the soaking solution on the integrity/stability of the crystal. Preferred methods are X-Ray-diffraction, or neutron-diffraction performed at in house-sources or at synchrotons and visual analysis of the crystal by means of UV/VIS- and IR-spectroscopy.

In a preferred embodiment, the decision to select a soaking solution is made after visual control and/or after X-ray-evaluation. In a preferred embodiment, all kinds of crystals or crystal remains are subjected to control by X-Ray-analysis since sometime crystals of bad appearance diffract nicely. In a preferred embodiment, only empty wells, where crystals dissolved or splintered completely are discarded.

In a preferred embodiment, more than one crystal are controlled. In a most preferred embodiment, crystal from at least 2 different biological macromolecules are controlled in the same composition of solution(s).

In a preferred embodiment, the biological macromolecule is a protein, preferably a binding protein, more preferably a receptor protein.

In another preferred embodiment the macromolecule is an RNA molecule. In a most preferred embodiment, the RNA molecule is a messenger RNA. More preferably, the mRNA comprises a riboswitch, more preferably the mRNA comprises an aptamer.

The visual inspection is preferably to see how the appearance of crystals change over time.

In a preferred embodiment, the selection of soaking solution compositions is done after visual analysis of the crystal. Examples of non-suitable soaking solution compositions are those which resulted in complete dissolution of crystals or in severe damage like splintering are discarded. The first group of soaking solutions selected after visual control can then be subject to X-ray analysis. The skilled person sets up all control and selection parameters using no more than common general knowledge. The method of controlling the crystal and selecting the suitable soaking solution(s) according to the method of the present invention do not require any particular knowledge beyond common general knowledge of an expert in protein crystallization. The parameters of the controlling method such as the maximum resolution, low mosaicity, intact crystal structures, quality of electron density, missing twinning and absence of diffraction signals caused by undesired ice crystals will be set up to allow the standard control of the crystal integrity and the subsequent decision to select the suitable soaking solution.

In a preferred embodiment, the data collected in the controlling step of the crystal are analyzed to select the soaking solution. The soaking solution which results in improvement of the crystal e.g. stability over time or at least no or minimal detrimental impact on the crystal are selected. The selection criteria can be established by the crystallographer using common general knowledge.

In a preferred embodiment, the crs is a salt solution different from original crystallization solution used for nucleation, growth and/or storage of the macromolecular crystal. The use of freshly prepared crs renders the process independent from the original crs. In a preferred embodiment, the salt of the crs is any salt that can be used for crystallization of biological macromolecules. Preferably, the salt of the solution is NaCl, MgCl₂ or KCl. In a different embodiment, the salt is not a chloride (CI) salt, in particular not NaCl. In a preferred embodiment, the same soaking solution(s) is/are suitably used for soaking crystals of different biological molecules, in particular different proteins.

In a preferred embodiment, more than one crystal are controlled. In a most preferred embodiment, crystal from at least 2 different biological macromolecules are controlled in the same composition of solution(s).

In a preferred embodiment, the cr, os, cs and/or w contain small molecules or preferably os contains small molecules.

In a preferred embodiment, the os contains small molecules as defined below and in particular small molecule fragments or molecular probes.

In a most preferred embodiment, small molecule concentrations are 10 times higher than the binding constant of the respective small molecule to the respective biological macromolecule are used for soaking. In a preferred embodiment, small molecules or a cocktail of small molecules are used in a concentration typically ranging from micromolar to molar, preferably in a range from 5 mM to 500 mM, per individual soaking solution. The concentration of drug-size small molecule is around at least 5-20 mM. The concentration of fragment is preferably of at least of 10-50 mM. In a preferred embodiment, high concentrations of the small molecules in particular in the os (as long as without detrimental effects on the crystals) of more than 500 mM and up to 1M concentration in the os is used

When the soaking solution contains small molecules, a preferred embodiment consists in further performing a stress test on the crystal regarding the tolerance of a biological macromolecular crystal towards changes introduced by the addition of small molecules. Such stress conditions occur upon introduction of pH-changing small molecules or are caused by changes in osmotic pressure induced by high concentrations of small molecules. Therefore, the tolerance of a biological macromolecular crystal is tested towards pH changes by addition of hydrochloridic fragments and fragments and/or molecular probes at different concentrations to the formerly identified soaking conditions containing biological macromolecular crystals.

In another aspect of the invention, the method is a method of small molecule screening for a biological macromolecular crystal wherein the screening is performed in a soaking solution selected according to the method of the invention of selecting a soaking solution.

In another aspect, the invention is an array arranged to perform the above methods of the invention.

The invention also concerns the soaking solution obtainable by any of the above method of selecting the composition of a soaking solution. In a preferred embodiment, the soaking solution of the invention Vos is more than 5%, preferably, more than 10%.

In a more preferred embodiment, the soaking solution obtainable by any of the above method of selecting the composition of a soaking solution does not contain Cl, in particular NaCl.

The invention also concerns the use of the selected soaking solution for small molecule screening for a biological macromolecular crystal.

In a preferred embodiment where several solutions are tested, preferably simultaneously, more than one solution is selected thus defining a range of volume/percentage for each component of the tested solution.

In a further particular embodiment of the present invention a biological macromolecular crystal is incubated for 1 minute or less to 72 h, preferably 24 h, in the soaking solution containing the small molecules or a cocktail of small molecules.

In another particular embodiment of the present invention the biological macromolecular crystal is vitrificated at cryogenic temperatures or alternatively subjected to a direct X-ray measurement at room temperature.

In an additional particular embodiment of the present invention the biological macromolecular crystal is stored at cryogenic temperatures in preferably liquid nitrogen and or helium or said biological macromolecular crystal is directly subjected to data collection by imaging techniques comprising X-ray, electron and neutron diffraction.

In the present invention individual soaking solution compositions suitable for a small molecule screening process of said biological macromolecular crystal means that soaking solution is judged as “suitable” in particular for a small molecule screening process if it allows to perform a “Hit picking” process wherein the binding of a given small molecule to the biological macromolecular crystal is defined as “hit.

In a most preferred embodiment, a soaking solution composition is suitable for a small molecule screening process of said biological macromolecular crystal if it enables the soaking of fragments and small molecule ligands into a biological macromolecular crystal resulting in highest resolution possible in diffraction experiments less than 2.8 Å preferably less than 2.5 Å in case of molecular probes and fragments and to less than 3.5 Å in case of drug-size molecules. Ideally, the biological macromolecular crystal shows minimal mosaicity, typically less than 0.8°; reveals an electron density which is then evaluated i.e. by an experienced crystallographer

Subject matter of the present invention is a method according to the above described invention, wherein said method is automated.

In a further embodiment, the subject matter of the invention concerns the soaking solution selected by any of the above embodiment of the method of selecting the composition of a soaking solution of the invention. In a preferred embodiment, the soaking solution comprises at least 5% of os.

In a further embodiment, the subject matter of the present invention is the use of the soaking solution selected by any of the above embodiment of the method of selecting the composition of a soaking solution for small molecule screening for a biological macromolecular crystal.

In a further embodiment, the subject matter of the invention is an array arranged to perform the method of the invention and an array containing the solution as prepared using the method of the invention, in particular the preparation step 1 of the above methods, or an array containing the soaking solution(s) selected by the method of the invention of selecting the composition of soaking solutions.

Another embodiment of the invention is an array arranged to perform the method, wherein said array comprises a first dimension of at least two individual soaking solutions (1 n to xn) and a second dimension of at least two individual soaking solutions (1m to ym) wherein each of said soaking solutions is located in a separated compartment of said array, and wherein each of said soaking solution comprises an organic solvent (os), a compatible solute (cs), a crystallization solution (crs) and water, and wherein the ratio of volume compatible solute Vcs to volume organic solvent Vos is the same within a series of soaking solutions in said first dimension, or, alternatively, the molar ratio of organic solvent (os) to compatible solute (cs) Mos/Mcs is the same within a series of soaking solutions in said first dimension, and wherein the individual soaking solutions of said first dimension comprise the same organic solvent and the same compatible solute within a series of said soaking solutions in the first dimension, respectively, and wherein the ratio of the volume of water Vw and the volume of the crystallization solution Vcrs is varied over the individual soaking solutions of the series in the first dimension and wherein one of the individual soaking solutions of the series in the first dimension has a minimal or zero Vcrs and a maximum Vw and another individual soaking solution of said first dimension has a minimal or zero Vw and a maximum Vcrs and wherein the other individual soaking solutions of the series in said first dimension take values of Vcrs and Vw in between the two before-mentioned values.

A further embodiment of the invention is an array, wherein 1 n to xn defines the numbers of individual soaking solution compositions in a range of between 1 (1n) to 1×106 (xn) and wherein the numbers comprise but are not limited to 1×106 or 1×105 1×104 or 768 or 384 or 96 or 48 or 24, however, the set-up of the array determines the numbers of individual soaking solutions.

A preferred embodiment of the invention is an array, wherein the array comprises a second dimension (dll) of a series of individual soaking solutions for a biological macromolecular crystal wherein the compatible solute is varied over said second dimension and the ratio of volume compatible solute Vcs to volume organic solvent Vos or, alternatively, the molar ratio of organic solvent (os) to compatible solute (cs) Mos/Mcs, may be different dependent on the compatible solute used in the series of soaking solutions in said second dimension.

Another preferred embodiment of the invention is an array, wherein in each of said individual soaking solutions in a series the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs: Vos).

A further preferred embodiment of the invention is an array wherein in each of said individual soaking solutions in a series the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10.

Another embodiment of the invention is the use of an Array for obtaining the solutions as described in the previous embodiments.

### Definitions

### Definition of Compartment

A compartment is a container and refers to a separate section or part in the array used in particular for culturing or screening. In the present invention a compartment can be a single well of an array or equally can refer to any container or section of an array or part in which experiments can be performed. This includes any types of wells from commercially available and custom made (i.e. printed in a 3D printer, etc.) plates of any suitable materials and any other devices that can be used for soaking and/or screening. The term “compartment” can also refer to parts or organelles of living or preserved cells which comprise but are not limited to mammalian, insect and bacterial cells or additionally to (bio-) chips, foils, tubular or diverse shaped tubes made from any suitable materials.

### Definition of Array

An “array” is one or a series of compartments or containers or comprising an arrangement of elements (i.e. crystals) in a pattern on a solid support and/or vessels. In the present invention, an array can also mean one single container/compartment. When the array is of one single compartment it can also refer to a tube or a well. Although the pattern of an array is arranged typically in a two dimensional space, in particular, a standard screening array or commercial array for high throughput screening composed of a multitude of individual compartments or wells forming lines on 2 different perpendicular axis. The pattern may also be arranged in a one (e.g. a tube or a single well or a single line of wells) or three dimensional space (e.g. the superposition of at least 2 arrays having two dimensions. However the present invention is not limited to a straightforward spatial organization. As an example, information technology systems can be used to generate logical arrays, in which the distribution, the specification and the location of the respective components in the array can be tracked (i.e. in a table/sheet), but are not characterized by a straightforward spatial organization. Furthermore, the term array can also refer to different formats, which include “chips” or “biochips.” Here, a range of low density defined as i.e. two to ten different addressable locations to high density i.e.one thousand or more addressable locations can be arranged in or on the array. It shall be mentioned that the shape and size of the array chip or biochip can be irregular and is not limited by any geometrical format, even though the typical chip array format is rectangular. The configuration of the array can comprise a bundling, mixing and or homogenous blending of a plurality of addressable locations, which are suitable for i.e. high-throughput handling and robotic delivery of reagents. The spatial organization of the arrays furthermore allows the detection and or quantification of a given signal and includes but is not limited to chemical luminescence, X-ray, confocal or deflective light gathering and laser illumination. Array formats comprise any appropriate format for culturing and soaking biological macromolecular crystals including but not limited to microarrays, microchips, arrays of biomolecules attached to multiwall plates and living organisms such as insect, bacterial and mammalian cells (“in vivo crystallization”). Further examples for an array format comprise serial crystallography, foils and tubular shaped devices on or in which crystals can be subjected to X-Ray measurements.

### Definition of Series of Individual Soaking Solutions

A series of individual soaking solutions refers to at least two individual soaking solutions which have several features in common. In particular the individual soaking solutions of series may preferably have at least one of their components in common e.g. the same organic solvent (os), a compatible solute (cs), and/or a crystallization solution (crs). The common features can be the same volume of 1, 2, 3 or more of the components, the ratio of the volume of some of the components to 1 or more components and/or the number of individual solutions.

When more than one series is prepared, one is called “first series” but it can also equally be named “benchmark” series. This series is conveniently called “first” as it is often distributed in the first line of an array. However, the so called first can equally be named a “reference” or “benchmark” series and is not necessarily distributed in the first line or column of wells of the array. Actually, this benchmark series is used as a reference for the preparation of the additional series according to the method of the invention. When more than one series is prepared, the so called first series or benchmark series can therefore be any one of the series which will then be used as benchmark for the rule-based method of the invention.

In a preferred embodiment, a series of at least two individual soaking solutions refers to the composition of at least two soaking solutions, which comprises the volumes of an organic solvent, a compatible solute, distilled water and a crystallization solution wherein in each series different compatible solutes are used.

In one embodiment, in each series the crystallization solution or a crystallization solution of some multiple of the original concentration is diluted in a serial dilution whereas the volume of distilled water increases proportionally. The volume of the compatible solute and the organic solvent and the ratio between all volumes in each series remains constant.

### Definition of Protein, Polypeptide and RNA

Polypeptide: The term “polypeptide” refers to any oligomeric arrangement of amino acids, which are typically but not exclusively covalently joined by peptide bonds and occur at variable lengths. In general, the term “polypeptide” refers to an amino acid sequence with less than 70 amino acids and a molecular weight of less than 70 kDa. The source of the polypeptide comprises a natural or unnatural origin, or a combination thereof including a naturally occurring polypeptide, a polypeptide produced by recombinant molecular genetic techniques, a polypeptide from a cell or translation system, or a polypeptide produced by cell-free synthetic means. The sequence of the amino acids in a polypeptide determines its structure and is not limited to full-length sequences, but can be partial or complete sequences. In general, the term “peptide” refers to a small polypeptide which consists of 2-25 amino acids in length, whereas a native polypeptide relates to a polypeptide having a sequence of amino acid residues which is identical to the one found in nature (e.g. the wild-type polypeptide). Moreover, a native polypeptide can have a natural origin and be isolated from its naturally occurring source (i.e. a plant cell or tissue) or be genetically engineered using recombinant genetic techniques. Post-translational modifications may or may not occur in native polypeptides depending on the naturally occurring wild-type polypeptides. The term polypeptide can also refer to a polypeptide fragment. As used herein, this term refers to any contiguous subset of the full-length polypeptide amino acid sequence. A polypeptide fragment or portion can be isolated from any domain of the polypeptide and is not limited to a specific length ranging from about 4 amino acids to up to a full-length polypeptide sequence. Furthermore, a polypeptide can possess or not possess any given biological or synthetic activity. The term “protein” refers to a polymer of amino acid which is linked together by peptide bonds. The term, as used herein, refers to proteins, polypeptides, and peptides of any size, structure, or function. As laid out in the definition of a polypeptide, a protein may have a natural origin, recombinant (i.e. biotechnologically produced in genetically modified organisms or transiently transfected cell cultures, with or without mutations, etc.), synthetic or comprise any combination of the above mentioned. Furthermore, the term “protein” refers to single molecules or to complexes of multiple molecules (complexes of proteins with i.e. other peptides, polypeptides and proteins). The term protein may also apply to amino acid polymers containing one or more amino acid residues of the artificial chemical analogue of a corresponding naturally occurring amino acid. Furthermore the term protein may also refer to prions which are misfolded proteins with the ability to transmit their abnormal shape onto harmless variants of the same protein.

RNA is a polymeric biomolecule consisting of ribose nucleotides (nitrogenous bases appended to a ribose sugar and attached by phosphodiester bonds) that forms strands of varying lengths and structural forms and conformations (for example: bulges and helices, circles etc.). The nitrogenous bases include adenine, guanine, cytosine and uracil. RNAs can be broadly classified based on the lengths of the chain of nucleotides and comprise small RNAs (200 or fewer nucleotides) and long or large RNAs (200 or more nucleotides). The group of small RNAs comprises but is not limited to transfer RNA (tRNA), 5S ribosomal RNA (rRNA), microRNA (miRNA), small interfering RNA (siRNA), small nucleolar RNA (snoRNA), Piwi-interacting RNA (piRNA), tRNA-derived small RNA (tsRNA), and small rDNA-derived RNA (srRNA), whereas the group of long or large RNAs comprises but is not limited to long non-coding RNA (lncRNA) and messenger RNA (mRNA). in particular mRNA comprising a riboswitch (see definition in the next paragraph). However, the term RNA may also refer to the genetic material (2 to 20 kb) of RNA viruses comprising double stranded RNA, single-stranded RNA(+/−), isometric particles with single-stranded RNA, pseudoviridae (i.e. Ebola, SARS, influenza, etc.). Also, the term RNA may refer to viroids or virusoide/satellite viruses. Viroids are single-stranded, covalently closed circular RNA molecules characterized by a small size (250-400 nucleotides), that do not encode for any proteins and lack a capsid (but contain a protein coat), whereas virusoides or satellite viruses refer also to circular subviral particles with single-stranded RNAs, but are dependent on viruses for replication and encapsidation. The genome of virusoide usually consists of several hundred nucleotides (200-400).

Riboswitches are regulatory segment of a messenger RNA molecule that binds a small molecule, resulting in a change in production of the proteins encoded by the mRNA and are often conceptually divided into two parts: an aptamer and an expression platform. The aptamer directly binds the small molecule, and the expression platform undergoes structural changes in response to the changes in the aptamer. The expression platform is what regulates gene expression. A preferred embodiment of the present invention is wherein the biological macromolecule is from mRNA comprising the aptamer of a riboswitch.

### Definition of Binding Site:

A macromolecular binding site refers to any position on a given macromolecule (such as a protein or mRNA etc.) that binds with specificity to another molecule. This binding partner is called i.e. a ligand and may include other proteins resulting in a protein-protein interaction or small molecules comprising i.e. enzyme substrates, hormones, allosteric modulators, second messengers, and inhibitors. Binding events may result in conformational changes of the respective macromolecule resulting in an alteration of its function or activity like in proteins and riboswitches in mRNA for instance. Most of such binding events are non-covalent reversible (transient), less often also covalent reversible or irreversible. Types of binding sites include active sites (orthosteric sites) and allosteric sites.

Active sites bind substrates to induce a chemical reaction as well as inhibitors that inhibit the respective reaction. Allosteric sites are regulatory sites that bind ligands that may enhance or inhibit a macromolecules function.

### Definition of Water:

Water refers to an inorganic substance, which generally comprises one oxygen and two hydrogen atoms connected via covalent bonds. In the present invention, the term water refers to but is not limited to i.e. the four different types of water used in the laboratory. Type I water is ultrapure and has low bacterial and organic levels, the sodium level is maximal 1 μg/L, the chlorides level is maximal 1 μg/L, total silica level is maximal 3 μg/L and the resistivity is usually >18 MΩ·cm. Type II water is pure water with a resistivity of >1 MΩ·cm and has low bacterial and organic levels, the sodium level is maximal 5 μg/L, the chlorides level is maximal 5 μg/L, total silica level is maximal 3 μg/L. Here, deionisation or (electrical) ion exchange removes ions from reverse osmosis water by the usage of synthetic resins. Type Ill water is less pure than type I and II water and produced using a reverse osmosis system and carbon filtration directly from tap water. Type Ill water has a resistivity of >4 MΩ·cm, the sodium level is maximal 10 μg/L, the chlorides level is maximal 10 μg/L and the total silica level is maximal 500 μg/L. Lastly, type IV water has a resistivity of >200 MΩ·cm, the sodium level is maximal 50 μg/L, the chlorides level is maximal 50 μg/L and the total silica level is not limited. However, the term water may also refer to feed water or so called raw or potable water from which the quality is dependent on its source. The term water used herein refers to examples like tap water, demineralized water, sterile filtered water, distilled water, DNase/RNase free water such as DEPC water, deionized, reverse osmosis water, industrial water and heavy water. It shall be mentioned that the term water refers to any kind of water suitable for the usage in the present invention.

### Definition of Soaking Solution

A soaking solution is any solution that serves as liquid environment of a biological macromolecular crystal that differs in its composition from the original environment in which the biological macromolecular crystal nucleated and grew. Usually it is derived from the original crystal growth environment which contains additionally organic solvents or compatible solutes and other components. The component of the soaking solution that is derived from the original growth environment may be of the same concentration of its individual components or of multiples of this concentration or diluted.

Soaking solutions are usually applied to introduce either small molecules as molecular probes, fragments and drug-size molecules to the crystal in order to elucidate binding events of respective small molecules in subsequent diffraction experiments. Furthermore, soaking solutions are applied to introduce heavy-atoms to the biological macromolecular crystals in order to obtain the phases for solving hitherto unknown structures in the course of subsequent diffraction experiments. Furthermore, they are applied to introduce cryoprotectants to mediate cryo-stress resistance, to prepare crystals for biotechnological applications (for example chromatographic applications or biosensors) and/or to improve crystal quality. The volume of a soaking solution ranges usually from μm to ml, typically between 1 to 10 μl. In case of automation volumes in the scale of nanoliters can be adjusted accordingly.

The volume of the individual soaking solutions ranges between pico- to milliliter.

Unless the volume is expressed specifically in standard measures of volume e.g. μm, ml, “V” such as in Vos, Vcs, Vcrs and Vw always refers to the proportion of the relevant component of the solution to the total volume (VT) of the solution ie. 100%. For instance Vos refers to the percentage/proportion of organic solvent (os) in the whole volume of solution e.g. Vos=10 means os is 10% of the total volume of the solution while Vos=10 μl means 10 μl of os solution.

In a preferred embodiment, the VT is VT=Vcrs+Vw+Vos+Vcs ie. the solution consists in a mixture of crs, w, os and cs. In some embodiment, Vcrs and/or Vw may or may not be 0%. Some of the solutions to be selected can therefore only contain os and cs, or os and cs and crs, or os and cs and water.

A preferred V_{crs} min is 0%.

A preferred V_{w} min is 0%.

A preferred volume of os, Vₒₛ is more than 5%, preferably at least 10%, or at least 15% or at least 20% more preferably between 5% to 25%.In another embodiment Vos is at least 0.5%.

A preferred volume of cs is preferably at least 5%, more preferably 10%, or at least 20%, at least 30, at least 40%, at least 50% and more preferably between 5% and 80%, most preferred 10% to 60%

### Definition of Small Molecule Especially Molecular Probe, Fragment and Drug-Size Small Molecule.

In the field of molecular biology and pharmacology the term “small molecule” refers to any low molecular weight organic compound with a molecular weight usually in the range of 100-500 Da, but can extend in special cases up to about 2000 Da (e.g. in case of macrolides or proteolysis targeting chimeras (PROTACs), in contrast to high molecular weight compounds like polymers as RNA, DNA, proteins or polysaccharides. Small molecules even comprise monomer or oligomer constituents of RNA, DNA, proteins (peptides) and polysaccharides as there are ribo- and deoxyribonucleotides, amino acids, mono- or oligosaccharides. Many small molecules regulate biological processes or are otherwise relevant in biological systems. As such, they can exert their effect for example as agonist and antagonist, as inhibitor or mediator of protein-protein interfaces, proximity or interaction, as regulator of biophysical properties like osmotic pressure etc. They can have a variety of functions in for example (but not limited to) signal transduction, metabolism, regulation of osmotic pressure, cryoprotection. They can have applications for example as drugs, molecular probes, starting points for drug discovery, serve as lead compounds, clinical candidates, as pesticides, cosmetics, food chemistry, fragrances, fungicide and many others. Their origin can be natural as secondary metabolites or synthetic as drugs and can have beneficial effects as therapeutics or detrimental effects like poisons. In drug discovery campaigns small molecules are used to screen for interactions with target macromolecules and may have or may not have binding affinity to respective targets or may or may not alter biological processes. They can be used as molecular probes or as small molecule fragments.

A “molecular probe” is a small molecule of 30-120 Da with an attached functional group (e.g. amino group, acid group, hydroxyl group etc.) used in molecular sciences such as biochemistry, structural biology or chemistry or medicinal chemistry to study the properties of other molecules especially biologically relevant macromolecules (RNA, DNA, proteins, peptides, polysaccharides) and to explore their interaction behavior. Therefore, a molecular probe can be a group of atoms or molecules used in molecular biology or chemistry to study the properties of other molecules or structures. If some measurable property of the molecular probe used changes when it interacts with the analyte (such as a change in absorbance), the interactions between the probe and the analyte can be studied. Radioactive DNA or RNA sequences are used in molecular genetics to detect the presence of a complementary sequence by molecular hybridization. In computational approaches molecular probes can even be artificial constructs that reflect predominantly one property, e.g. of a hydrogen-bond donor or a lipophilic probe. If some measurable property of the applied molecular probe changes when it interacts with the analyte (such as a change in absorbance or the interaction geometry), the interactions between the probe and the analyte can be studied. In molecular sciences such as structural biology (e.g. crystallography) molecular probes are used to map properties of binding sites of macromolecular targets as there are: hydrophobic, hydrophilic properties H-bonding patterns induced polarization effect resulting shifts of protonation states, conformational changes and induced molecular adaptations. Furthermore, molecular probes provide information about putative pharmacopohore patterns, which help to define the requirements which set of small molecules is applied in screenings. Furthermore, a molecular probe can be used to perform stress tests for example to assess a macromolecule's or a macromolecular crystal's tolerance towards for example organic solvents or pH changes. Molecular probes enable to indirectly study the properties of compounds and structures, which may be hard to study directly. The choice of molecular probe will depend on which compound or structure is being studied as well as on what property is of interest.

The term “small molecule fragment,” refers to a small molecule (e.g. ≤250 kDa in size) typically having a KD ranging usually from low one digit-micromolar up to two-digit millimolar affinity. A small molecule fragment is typically 120-250 Da in size and serves as starting points for optimization by chemical synthesis for example to create ligands such as inhibitors, agonists or antagonists. The small molecule fragment can be, for example, a peptide, any organic molecule or natural product, a short nucleic acid sequence (e.g., DNA, or RNA type), and/or any other ligand including solvent molecules and cryo-buffer ingredients capable of binding to the macromolecular target. The “target” can be any macromolecular system with a biological function or macromolecular crystal system based on amino acids, nucleic acids as monomeric building blocks and additional sugar or lipid moieties; for example, enzymes, soluble proteins or membrane-bound proteins such as soluble or membrane-bound receptors or ion channels, RNA and DNA molecules or sugar-based macromolecules.

“Drug-size molecules” are organic compounds of 250-2000 Da, that may or may not exert a therapeutic effect as drug or lead candidate. Exerting such an therapeutic effect they serve the modulation of some kind of biological activity like a macromolecule's activity for example as agonist or antagonist or as catalyst like in targeted protein degradation. Drug-size molecules comprise, but are not limited to, drugs, agrochemicals, aromatic substances or fragrances.

One embodiment of the method of the present invention is to test a collection of small molecules in a crystal system of a biological macromolecule in order to separate small molecules which interact with the respective biological macromolecule from the small molecules of the collection that do not interact with respective biological macromolecule.

Typically, in the array and method of the invention, the small molecules are dissolved in the os.

### Definition of Organic Solvent (Os)

A solvent is a substance capable of dissolving other substances. The term organic solvent (os) refers to any carbon-based solvent, protic or aprotic, of low reactivity that is capable of dissolving or dispersing one or more other substances. Carbon-based means that these solvents at least contain one carbon atom. Furthermore, they contain most often at least one hydrogen atom. The terms “liquid carbohydrates” and “carbon-based solvents” have the same meaning and are used interchangeably.

Examples of suitable organic solvents are a straight or branched chain monohydric aliphatic alcohol containing from 1 to 7 carbon atoms, such as ethyl alcohol or isopropyl alcohol; a dihydric aliphatic alcohol containing from 2 to 7 carbon atoms, such as hexylene glycol; a monoalkyl ether of an aliphatic dihydric alcohol containing a total of 3 to 6 carbon atoms, such as the monomethyl, -ethyl and -butyl ethers of ethylene glycol; or a dialkyl ketone containing a total of 3 to 5 carbon atoms, such as acetone. Other solvents that can be used are benzyl alcohol or phenyl ethyl alcohol. Preferred solvents are ethyl alcohol, hexylene glycol, the monomethyl ether of ethylene glycol, and acetone. Mixtures of solvents can also be used.

Organic solvents may be selected from the preferred group comprising but not limited to: methylsulfinylmethane (dimethyl sulfoxide (DMSO)) and derivatives, especially phospoderivatives, ethyl acetate and derivatives, methanol, ethanol, propan-1-ol, propan-2-ol, N,N-dimethylformamide, oxolane (tetrahydrofuran), ethoxyethane (diethyl ether), crown ethers, 1,4-dioxane (dioxane), furan and others suitable to dissolve small molecules for protein crystal-based screenings.

According to the present invention, mixtures of single organic solvents selected from the above mentioned list of preferred organic solvents can be used. Thus, the term organic solvents according to the present invention may refer to a mixture of organic solvents. According to the present invention, mixtures of single organic solvents selected from the above mentioned list of preferred organic solvents can be used in the array.

In a preferred embodiment, small molecules are dissolved in the os.

### Definition of Compatible Solute (Osmoprotectant, Osmolyte or Cryoprotectant):

Compatible solute (cs), osmoprotectants or osmolytes comprise organic compounds of natural or synthetic origin that prevent proteins from precipitation or denaturation under stress conditions and keep polypeptides, proteins and RNA regarding their spatial organization/conformation stable and soluble. Such stress conditions include elevated or lower temperature changes (including cryo-conditions), changes in osmotic stress, presence of protein denaturing compounds like urea, drought, radiation, radicals, poisons as well as pressure. Natural compatible solutes are of high solubility in water and of low toxicity towards organisms even at high concentrations thereby acting as osmolytes and help organisms survive extreme osmotic stress. Compatible solutes may be selected from the group consisting of methylamines, polyols (including polymers, mono-, di-, trisaccharides, cyclitols), amino acids and others.

Compatible solutes have been shown to stabilize macromolecule conformation and in particular to induce the adoption of distinct conformations in proteins and RNA molecules.

In a preferred embodiment, the compatible solute is selected from the group consisting of methylamines, polyols including polymers, mono-, di-, trisaccharides, cyclitols, and amino acids. In a most preferred embodiment, the cs is poly(oxyethylene) of a molecular weight of 400 Da (PEG400) or 3350 Da (PEG3350), 2-Methylpentane-2,4-diol (MPD), ethane-1,2-diol (ethylene glycol, EG) and/or propane-1,2,3-triol (glycerol).

In some embodiment, the cs is liquid and is added to the soaking solution at the volume Vos. Examples of liquid cs are poly(oxyethylene) of a molecular weight of 400 Da (PEG400), propane-1,2,3-triol (glycerol), ethane-1,2-diol (ethylene glycol), 2-Methylpentane-2,4-diol (MPD).

In some embodiment, the cs is solid and is added to the soaking solution dissolved in water whereby Vos refers to the volume of the respective cs dissolved in water. Examples of solid cs are N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-aminoethanesulfonic acid (taurine), 2-aminoethanesulfinic acid (hypotaurine), (2S)-pyrrolidine-2-carboxylic acid (proline).

In an additional particular embodiment of the invention the preferred group of methylamines comprises N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine) as well as respective possible stereoisomers and derivatives.

In a further additional particular embodiment of the invention the preferred group of amino acids comprises 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[[(1R)-1-carboxyethyl]amino]-5-(diaminomethylideneamino)pentanoic acid (octopin) as well as respective stereoisomers and derivatives.

In an additional particular embodiment of the invention the preferred group of polyols comprises poly(oxyethylene), monosaccharide, disaccharide, trisaccharide, cyclitols and derivatives of former molecules.

In an additional particular embodiment of the invention the preferred group of poly(oxyethylene) comprises polyols of diverse molecular weights (for example polyethylene glycol 400), propane-1,2,3-triol (glycerol), ethane-1,2-diol (ethylene glycol), 2-Methylpentane-2,4-diol (2-Methylpentane-2,4-diol, MPD), (3R,4S,5S,6R)-2-(2,3-dihydroxypropoxy)-6-(hydroxymethyl)oxane-3,4,5-triol (1-glucosylglycerol), PEG 3350. (also referred as 3350 or macrogol 3350). In a preferred embodiment, PEGs are of molecular weights ranging between 200-20000 g/mol and includes but is not limited to poly(oxyethylene) (200 g/mol, 300 g/mol, 400 g/mol, 550 g/mol, 600 g/mol, 1000 g/mol, 1500 g/mol, 2000 g/mol, 3350 g/mol, 4000 g/mol, 5000 g/mol, 6000 g/mol, 8000 g/mol, 10000 g/mol, 20000 g/mol).

In a preferred embodiment, the compatible solute is a mix of at least 2 poly(oxyethylene)s preferably PEG400 and 3350.

In an additional particular embodiment of the invention the preferred group of monosaccharides comprises (preferably: (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside),

In an additional particular embodiment of the invention the preferred group of disaccharides comprises (β-D-Fructofuranosyl α-D-glucopyranoside (sucrose), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose)),

In an additional particular embodiment of the invention the preferred group of trisaccharides comprises (preferably (2R,3R,4S,5S,6R)-2-[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol (raffinose)), sugar alcohols (preferably (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol), (2R,3S,4R,5S)-hexane-1,2,3,4,5,6-hexol (galactitol)),

In an additional particular embodiment of the invention the preferred group of cyclitols comprises (preferably (1S,2S,4S,5R)-6-methoxycyclohexane-1,2,3,4,5-pentol (pinitol)),

In an additional particular embodiment of the invention the preferred group of derivatives of former molecules comprises preferably mannose derivatives as fiorin ((2R)—O2-(β-D-mannopyranosyl)glyceric acid and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose as well as respective stereoisomers and derivatives.

In another particular embodiment of the invention the preferred group of other molecules comprises urea and derivatives, 1-methyl-2-pyridinecarboxylic acid (homarine), methylsulfonium solutes as 3-dimethylsulfoniopropanoate (Dimethylsulfoniopropionate), and methylsulfinylmethane as dimethyl sulfoxide and other compounds with traits of compatible solutes according to definition as well as respective stereoisomers and derivatives.

### Definition of Crystallization Solution (Crs)

The crystallization solution (crs) refers to any solutions used for the preparation of a crystal at any point in time of the course of any standard crystallization process, in particular for nucleation, growth and/or storage of the crystal in conditions suitable to preserve the integrity of the crystal for any further uses. As a way of examples, crs of the invention can be defined under various terms known in the art such as mother liquor, medium of nucelation, medium of crystal growth, reservoir solution, storage solution. In particular, crystallization solutions comprise the mother liquor and other solutions like reservoir solutions at any state in the process of crystallization.

In a preferred embodiment, the crystallization solution is obtained from the crystallization process of the biological macromolecular crystal and typically comprises pH-maintaining buffer substances, precipitants and additives. In the present invention, crs refers to any solution that is used for the preparation of the crystallization of macromolecules using any standard method of crystallization known in the art. Ducruix, A. & Giegé, R. (eds.) Crystallization of Nucleic Acids and Proteins (Oxford University Press, Oxford, 1999); 1. Gavira, J. A. Current trends in protein crystallization. Archives of Biochemistry and Biophysics 602, 3-11 (2016). 1. McPherson, A. & Gavira, J. A. Introduction to protein crystallization. Acta Crystallographica Section F Structural Biology Communications F70, 2-20 (2014).

In other embodiment, the crystallization solution also refers to any solutions that are not directly obtained from the crystallization process, medium of nucleation and growth of the crystal. Crystallization solution also refers to solutions freshly prepared and used as surrogate of the medium of nucleation and growth of the crystal. In particular any solutions used to store the crystal in a suitable environment to preserve the crystal structure or to maintain the crystal integrity.

Therefore, the crystallization solution (crs) refers both to 1) the crystallization solution crs that is obtained from the crystallization process of the biological macromolecular crystal and 2) to a freshly prepared solution of organic or inorganic monovalent, bivalent or trivalent salts such as chloride salts, preferably NaCl, MgCl₂ or KCl.

In the latter embodiment, the crystallization solution (crs) is also called an equivalent salt solution and refers to any solutions that are not directly obtained from the crystalization process, medium of nucleation and growth of the crystal. Crystallization solution also refers to solutions freshly prepared and used as surrogate of the medium of nucleation and growth of the crystal.

The crs can equally refer to a freshly prepared solution that is not extracted from the crystallization process and the solution that was used to grow crystals. In this embodiment, the freshly prepared crs typically consists in an aqueous solution of salts and/or precipitants that is not the original crs but nevertheless exerts an equivalent effect in a soaking solution in terms of maintaining the osmotic net pressure, protein solubility, permittivity and the proteins conformation and, as such, preserves the crystals integrity. Typically, the freshly prepared aqueous solution consists of organic or inorganic monovalent, bivalent or trivalent salts, in particular sodium; chloride, potassium; chloride, diazanium; sulfate (ammonium sulfate), sodium; acetate (sodium ethanoate), sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate)), dilithium; sulfate, lithium; chloride, lithium; acetate, lithium; formate, lithium; nitrate, magnesium; diacetate, sodium; nitrate, sodium; formate, and amphoteric salts, precipitants as poly(oxyethylene), in particular PEG3350, PEG4000, PEG8000, and others as MPD.

Advantageously, the freshly prepared crs renders the process independent from the original crs. Another advantage is that the same soaking solution can be suitably used for soaking crystals of different biological molecules, in particular different proteins. Preferably, when a salt solution of freshly prepared crs is used in the soaking solution, the cs is a mixture of PEG, more preferably PEG400 and PEG3350.

The mother liquor is any solution containing concentrated proteins, polypeptides or RNAs for crystallization. In addition, it might contain pH-maintaining buffer substances, precipitants and additives. A reservoir solution is any solution that contains pH-maintaining buffer substances precipitants and additives but usually no protein that in respect to some vapor-diffusion techniques serves to drive the proteins in the mother liquor into supersaturation. Supersaturation is a non-equilibrium condition in which some quantity of the proteins, polypeptides or RNA is present in excess of the solubility limit. Equilibrium is re-established in the process of crystallization. Supersaturated solutions are produced by modification of properties of undersaturated solutions in order to reduce the ability of the medium to solubilize the macromolecule. Techniques for achieving supersaturation comprise bulk crystallization, batch method in vials, microbatch under oil, controlled evaporation, bulk dialysis, concentration dialysis, microdialysis, free-interface diffusion, counter-diffusion in capillaries, liquid bridge, vapor diffusion on plates (sitting drop), vapor diffusion in hanging drops, sequential extraction, pH-induced crystallization, temperature-induced crystallization, crystallization by effector addition, in vivo crystallization. Since the compositions of mother liquor and reservoir solution are altered in the process of the afore mentioned techniques (for example by dialysis or vapor diffusion) the term mother liquor and reservoir solution, compiled in the term crystallization solution, apply to respective solutions at any point in time of the course of the crystallization process.

The crystallization solution can be of starting concentrations that are multiples of the original concentration of the crystallization solution as it is used in the crystallization process. The multiples can be whole number multiples, for example 3-fold, or fraction number multiples, for example 3.5-fold or 0.8-fold. In a preferred embodiment the crystallization solution is used in a 1-fold concentration of the original crystallization solution as it is used in the crystallization process. This means that the starting concentration of the crystallization solution used according to the present invention is of the same concentration as the crystallization solution used in the course of a crystallization process.

### Definition of Precipitant

A precipitant is a chemical reagent that mediates a transformation process in which a dissolved substance like a biological macromolecule (e.g. a protein or RNA molecule) changes into an insoluble solid. The precipitation occurs when the concentration of the substance to be precipitated exceeds its solubility. In the field of crystallography the usage of precipitants aims at forming solids of biological macromolecules in terms of crystals of respective biological macromolecules, but oftentimes, if not successful, result in the formation of amorphous precipitates.

### Definition of Osmotic Net-Pressure Condition

The transfer of a protein crystal from an original crystallization environment to a soaking environment results in multiple redistributions of the solution's components i over the crystal surface along occurring short-term concentration gradients. The result is an influx of components from the soaking environment into the crystal if the according component's concentration in the soaking environment is higher than the component's concentration inside of the crystal's solvent channels. On the other hand, the result is an outflow of components from the crystal's solvent channels to the soaking environment if the component's concentration inside of the crystal's solvent channels is higher than in the soaking environment. These concentration differences result in redistributions along concentration gradients causing osmotic pressures that add up to an osmotic net-pressure which can either result in a compression or an extension of the crystal. In both cases crystals can crack. In order to maintain a crystal in a condition suitable for crystallographic analysis after soaking the osmotic net pressure π^{net} over the crystal's surface must not exceed x Pa in order to prevent the crystal's destruction:

$${Π}^{net} = {\sum }_{i} {Π}_{i} = 0 ∓ x$$

x is an empirical threshold that depends on the individual protein of interest and most likely its packing and defines a cut-off for a tolerable osmotic net-pressure.

### Definition of Protein Solubility Condition

The transfer of a protein crystal from an original crystallization environment to a soaking environment introduces changes in water activity, salt activity and activity of additives. This will affect the native protein's solubility S which results in the crystals dissolution if the solubility of the biological macromolecule is sufficiently increased in the soaking environment compared with the original crystallization solution. Therefore, in order to maintain the crystal's integrity upon the transfer from a crystallization environment to a soaking environment its soaking environment must be adjusted, such that its solubility S suffices:

S_{SS}^{Protein}≤S_{CS}_{_{eq}}^{Protein}

and for the respective crystals the following is given:

ΔGₜᵣ^{crys}≥0

with SS denoting the crystal's solubility S in the soaking solution, CS_{eq} denoting the crystal's solubility in the original crystallization solution at equilibrium between crystallization solution and crystal) and

Gₜᵣ^{crys} is the free energy upon transfer of the crystal from the original crystallization environment to the soaking environment that must be unfavorable in order to prevent the crystal's dissolution.

### Definition of Protein Conformation Condition

Upon the transfer of a biological macromolecular crystal from its original crystallization solution to a soaking environment new components are introduced to the crystal by means of the soaking solution that are not present in the crystallization solution. This especially concerns the introduction of organic solvents, compatible solutes and finally small molecules from screening libraries. Such components can cause denaturation of the individual biological macromolecules which means a perturbation, a partial or a complete loss of the macromolecule's native conformation. The loss of the native conformation may result in the loss of the macromolecular crystal's integrity and following that in its dissolution in its (partially) denatured form. Therefore, the effect of denaturing components must be compensated by structure-maintaining components. At concentrations suitable for screenings of small molecules organic solvents are used at concentrations that usually exert denaturing effects on biological macromolecules. Compatible solutes are natural compounds that can compensate this effect and preserve the biological macromolecule's native conformation. These molecules originated from organisms whose evolutionary history required protective agents to tolerate elevated stress conditions like drought, cold, high salt or urea concentrations. Their general effect is to cause preferential hydration of the macromolecules. Preferential hydration means that the protein interacts preferably with water instead of the compatible solute which, in turn, is excluded from the immediate protein surface. Denaturing reagents in contrast interact preferably with the biological macromolecule itself, especially with a protein's backbone. This results in an exclusion of water from and an enrichment of the denaturing reagent in the immediate surrounding of the macromolecule. Preferential interaction of a biological macromolecule with water exerts the hydrophobic effect that results in a folding reaction of an according biological macromolecule that gives rise to its native conformation. Displacement of water by a denaturant exerts the opposite effect. Consequently, the effect of a denaturant can be compensated by the use of a suitable compatible solute which enhances the hydration state of the biological macromolecule. This stabilizes the native conformation of individual biological macromolecules in the crystal lattice and consequently the crystal lattice's integrity. Since individual biological macromolecules interact differently with organic solvents and compatible solute a suitable combination of both must be identified. The here presented technology implements this condition by means of a systematic assessment of suitable combinations. The condition can be subdivided into two sub-conditions:
a. Precipitant condition

ΔGₜᵣ^{nat}=ΔGₜᵣ^{ASS}+ΔGₜᵣ^{BB}≥0
ΔGₜᵣ^{nat} is the free energy of the transfer of a native protein dissolved in the original crystallization solution (where it is after crystallization in equilibrium with the protein incorporated in the crystal) to a soaking solution which must equal zero or must be unfavorable, since the soaking solution must not increase the proteins solubility. Two aspects contribute to ΔGₜᵣ^{nat}: ΔGₜᵣ^{ASS} is the contribution to the free energy upon this transfer that arises, in case of a protein, from the amino acid side chains exposed on the native protein's surface and ΔGₜᵣ^{BB} is the contribution arising from the solvent accessible portion of the backbone of the native protein.
b. Stabilizing condition

ΔG_{N→D}^{SS}−ΔG_{N→D}^{CS}≥0
ΔG_{N→D}^{SS} is the change in free energy of unfolding in a soaking environment and ΔG_{N→D}^{CS} is the change in free energy upon unfolding in the crystallization-environment. If the difference is greater than zero the native conformation in the soaking solution is favored.

### Definition of Permittivity Condition

The permittivity is a measure for the electric polarizability of a dielectric. Upon the transfer of a protein crystal from an original crystallization solution to a soaking environment the permittivity of the solution may be changed due to the introduction of organic solvents, compatible solutes and small molecules for small molecule screenings. Because of multiple redistributions of these components the permittivity inside of a crystal may be different after the transfer and re-equilibration of the crystal. That means that either the original crystallization solution or the soaking solution may polarize more or less in response to an electric field than the other. Since the crystallization process requires screening of charges of the macromolecules by means of ions from the crystallization solution and consequently a retention of ions upon crystal formation a change in the permittivity ∈ more than y can result in a loss of the crystal's electrostatic setup.

∈_{CS}^{total}=∈_{SS}^{total}∓y

y is an individual threshold that depends on the individual type of macromolecular crystal and most likely its packing and defines a cut-off for a tolerable change in permittivity ∈.

### Definition of the Method for Controlling the Biological Macromolecular Crystal and Selection of the Suitable Soaking Solution

In the present invention, the step of controlling the crystal can be performed by any of the method available to the skilled person to evaluate the presence and/or aspect of the crystal and suitability for further analysis such as X-ray-diffraction analysis. The step of controlling the crystal refers to assess the survival of at least one biological macromolecular crystals in a particular soaking solution in terms of their presence and to measuring the impact of the soaking solution on the integrity/stability of the crystal. Preferred methods are X-Ray-diffraction, or neutron-diffraction performed at in house-sources or at synchrotons and visual analysis of the crystal by means of UV/VIS- and IR-spectroscopy.

In particular embodiment of the present invention the crystal is controlled and the soaking solution is subsequently selected by the use of routine technics such as standard X-Ray analysis and/or by visual control under a microscope. In a preferred embodiment, visual control will be followed by X-ray analysis.

In a preferred embodiment, more than one crystal are controlled. In a most preferred embodiment, crystal from at least 2 different biological macromolecules are controlled in the same composition of solution(s).

In a fully automated setup, a robot would grab the experimental plate, an artificial intelligence would identify crystals in a well (or discard the according well if the crystals are dissolved or in a bad condition) and the chosen crystals are transferred automatically to an X-Ray beam. Preferably, the ultimate decision is made by X-Ray-evaluation. Also, the visual inspection can serve to identify crystals in the well of an experimental plate that are worthwhile to subject to X-Ray-analysis. In a preferred embodiment, all kinds of crystals or crystal remains are subjected to control by X-Ray-analysis since sometime crystals of bad appearance diffract nicely. In a preferred embodiment, only empty wells, where crystals dissolved or splintered completely are discarded.

Visual inspection can be done under a microscope or using UV/VIS- or IR-spectroscopy to identify the surviving crystals that can be subjected to X-Ray-analysis.

The visual inspection is preferably to see how the appearance of crystals changes over time. This can be useful to estimate the maximum soaking time.

The visual data can be collected in order to correlate osmotic conditions in the array with cracking behavior etc.

In a preferred embodiment, a first selection of soaking solution compositions can be done after visual analysis of the crystal. Soaking solution compositions that resulted in complete dissolution of crystals or in severe damage like splintering are discarded. The first group of soaking solutions selected after visual control can then be subject to X-ray analysis. This concerns crystals from soaking solution compositions that, under visual analysis, show no signs of damage (that is sharp edges and smooth faces, no cracks, visible color under polarized light) and signs of moderate damage that does not compromise the general possibility to transfer and subject the according crystals to x-ray or neutron-diffraction experiments. With the X-ray- or neutron-diffraction analysis, the skilled person sets up all parameters using no more than common general knowledge.

The method of controlling the crystal and selecting the suitable soaking solution(s) according to the method of the present invention do not require any particular knowledge beyond common general knowledge of an expert in protein crystallization. The parameters of the controlling method such as the maximum resolution, low mosaicity, intact crystal structures, quality of electron density, missing twinning and absence of diffraction signals caused by undesired ice crystals will be set up to allow the standard control of the crystal integrity and the subsequent decision to select the suitable soaking solution.

For visual and X ray controls the parameter can be conveniently reported in a template like in FIGS. 6-7 and 10-11.

For visual inspection/control, the observer notes the presence or absence of minimal fractures, cracks, if no or minimal dissolution, sharp crystal edges will be evaluated, color s in polarized light etc. The selection criteria, for visual inspection in particular, can be based on the presence or absence of cracks or on the type of cracks in the crystal. Typically, the types of cracks can be classified (longitudinal, lateral, crisscross cracks). The appearance of the crystals surface (e.g. smooth, rough) or edges (e.g. sharp, roundish) and their behavior in polarized light (strong color, some color, no color) are standard criteria used to measure the impact of the soaking solution composition on the crystal and to select the soaking solution(s) which best preserve the integrity of the crystal. In the most cases not all of the crystals survive in all the conditions. Some crystals completely dissolve over time or are damaged while the integrity of other crystals is fully conserved.

The X-ray examination can take place with commercially available x-ray source as well as at a synchrotron. Commercially available x-ray sources are much slower in data collection. For that reason, at least two pictures at different crystal orientations, typically 0° and 90, are taken in order to safe time. These images are a sufficing basis for an evaluation of the crystals quality after subjecting them to according soaking solutions in terms of resolution, and mosaicity, the appearance of artifact like data originating from unwanted ice crystals in the protein crystal and sometimes a well-known phenomenon called “twinning”.

For the x-ray examination the crystals are preferably removed from the cryo-vials or any other storage containers and mounted on an x-ray or neutron diffraction machine for instance in a so-called cryo-stream that maintains the cryogenic temperature around the protein crystal. In case of x-ray studies at room temperature, no cryo-stream is required. The skilled person will typically define the standard criteria commonly used by the expert in the art and for instance the highest resolution should be set at less than 2.8 Å, more preferably 2.5 Å; low mosaicity should typically be lower than 0.8° (depending on detector), no or minimal ice rings, high-quality electron density, minimal twinning.

The data collected in the controlling step of the crystal are analyzed to select the soaking solution. The soaking solution(s) which results improved the crystal's quality in terms of the quality criteria or the results of no or minimal detrimental impact on the crystal is/are selected. The selection criteria can be established by the crystallographer using common general knowledge.

Data collection at a synchrotron is much faster. In this case, complete data sets are collected and evaluated since the appearance of the so called “electron density” becomes an additional criterium of validation judged by an experienced crystallographer as suitable or not suitable.

In a particular embodiment of the present invention the biological macromolecular crystal is controlled at least once in a timeframe from 0 h to several weeks, preferably after 0 h, 1 h and 24 h, by inspection in particular by x-ray in combination with preferably a visual inspection. The repeated inspection over time serves to determine the maximal amount of time for soaking experiments that maintain suitable diffraction quality in diffraction experiments.

Based on the data collected by X-Ray, neutron diffraction and/or visual analysis a decision is made on which soaking solutions is selected for further application in particular for small molecule fragment screening, for molecular probe analysis, screening for more drug-size small molecules, to validate HTS-hits or hits from biophysical screenings.

In contrast to traditional optimizations of soaking solutions which result in one single soaking condition, the method of the invention usually results in a spectrum of soaking conditions. From this spectrum the experimenter can further select within the selected spectrum the very best soaking solution to address any detrimental effects on the crystal that may be caused by for example respective utilized small molecule fragments or pH changes resulting from their utilization etc.

In a preferred embodiment, a further method of control which consists in performing stress tests on the crystals can be used. The method is particularly used to evaluate the tolerance of a biological macromolecular crystal to changes introduced by the addition of small molecules.

Typically the stress test will be performed as a further step following any of the above mentioned method of controlling the crystal selection. The stress tests can typically be used to fine-tune the selection of a first spectrum of soaking solutions first controlled and selected using X-Ray, or neutron diffraction, performed at inhouse sources or at synchrotons and visual analysis of the crystal.

For the stress tests, the volume and components of the selected solutions are conserved but the os contains small molecules called stress probes. Such stress conditions occur upon introduction of these small molecules which results in pH-changes or in changes in osmotic conditions using high concentrations of small molecules. The stress test typically consists in the addition of for example hydrochloridic fragments/probes for stress tests and fragments dissolved at different concentrations in the organic solvent of the formerly selected soaking solutions.

### Definition Aqueous Solution

The term “aqueous” describes a system that involves water and the term “solution” a system in which a substance, the solvent, dissolves another substance, the solute. An aqueous solution, therefore, is a is a solution of the solvent water of another substance, the solute. Polar or hydrophilic solutes are more soluble in water, for example salts, while hydrophobic or apolar solutes are only soluble in traces (oils). Nevertheless, traces of apolar substances can have a severe impact on the aqueous solution's traits.

The following embodiments are subject of the present invention:
1. Method of selecting a soaking solution suitable for soaking the crystal form of a biological macromolecule wherein the solution comprises an organic solvent (os), a compatible solute (cs), and a crystallization solution (crs) and/or water (w), comprising the steps of
   1) preparing the soaking solution by mixing os, cs, crs and w
   2) transferring at least one crystal of the biological macromolecular crystal into the solution prepared in step 1) or transferring the solution prepared in step 1) into a compartment comprising at least one crystal of the biological macromolecular crystal
   3) controlling the crystal
   4) selecting the soaking solution which is suitable for soaking the crystal.
2. Method of selecting a soaking solution for soaking the crystal form of a biological macromolecule wherein the soaking solution comprises an organic solvent (os), a compatible solute (cs), and a crystallization solution (crs) and/or water (w); comprising the steps of
   1) preparing a first series of 1 n to xn individual soaking solutions by mixing os, cs, crs and w wherein:
   a) x is at least 2
   b) each soaking solutions of the first series comprises the same organic solvent (os), the same compatible solute (cs), and the same crystallization solution (crs), and
   c) Vos and Vcs are the same in each solution, and
   d) Vcrs and Vw are changed in each solution, and
      in the solution 1n, Vcrs is the minimum Vcrs (Vcrsmin) and the Vw is the maximum Vw (Vwmax), and
      in the solution xn, Vw is the minimum Vw (Vwmin) and the Vcrs is the maximum Vcrs(Vcrsmax), and
      when x>2, in any additional solutions between 1 n and xn, the Vcrs is varied between Vcrsmin and Vcrsmax and, inversely, Vw is varied between Vwmin and Vwmax
   2) transferring at least one crystal of the biological macromolecular crystal per compartment into x compartments, each compartment comprising one of the solutions prepared in step 1); or transferring each solution prepared in step 1) into x compartments, each compartment comprising at least one crystal of the biological macromolecular crystal from its crystallization solution.
   3) controlling the crystal in each compartment
   4) selecting the soaking solution(s) which is/are suitable for soaking the crystal.
3. Method of selecting the composition of a soaking solution of any of embodiment 1 to 2 further comprising the step of
   1) preparing m series of 1 n to yn individual soaking solutions by mixing os, cs, crs and w wherein m is at least 1 and wherein in each of the m series
   a) y is at least 2
   b) os and crs are the same as in the first series, and
   c) Vos and Vcs are the same as in the first series
   d) the cs is changed and is different from the cs of the first series, and
   wherein in each individual solutions of each of the m series
   e) Vos and Vcs are conserved, and
   f) Vcrs and Vw are changed in each solution, and
      in the solution 1n, Vcrs is the minimum Vcrs (Vcrsmin) and the Vw is the maximum Vw (Vwmax), and
      in the solution yn, Vw is the minimum Vw (Vwmin) and the Vcrs is the maximum Vcrs(Vcrsmax), and
      when y>2, in any additional solutions between 1 n and yn, the Vcrs is varied between Vcrsmin and Vcrsmax and, inversely, Vw is varied between Vwmin and Vwmax
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
4. Method of selecting the composition of a soaking solution of any of the embodiments 1 to 3 wherein the crs is a salt solution different from original crystallization solution used for nucleation, growth and/or storage of the macromolecular crystal, and wherein the salt of the crs is any salt that can be used for crystallization of biological macromolecules.
5. Method of selecting the composition of a soaking solution of embodiment 4 wherein the salt is NaCl, MgCl2 or KCL.
6. Method of selecting the composition of a soaking solution of embodiment 4 wherein the salt is not a chloride (Cl) salt, in particular not NaCl.
7. Method of selecting the composition of a soaking solution of any of the embodiments 1-6 wherein the percentage of the total volume (VT) of each solution of the first series is VT=Vcrs+Vw+Vos+Vcs=100%.
8. Method of selecting the composition of a soaking solution of any of the embodiments 1-7 wherein the step of controlling the crystal is performed by X-Ray-diffraction neutron diffraction, absorption spectroscopy or reflectance spectroscopy in ultraviolet and visible spectral regions, infrared spectroscopy and/or visual analysis.
9. Method of selecting the composition of a soaking solution of any of the embodiments 1-8 wherein the os contains small molecules including small fragments and small probes.
10. Method of small molecule screening for a biological macromolecular crystal wherein the screening is performed in a soaking solution selected according to the method of embodiment 9.
11. Soaking solution obtainable by the method of selecting the composition of a soaking solution as described in any of the embodiments 1-9.
12. Soaking solution as described in embodiment 11 wherein Vos is at least 5%.
13. Use of the soaking solution as described in any of embodiment 11 or 12 for small molecule screening for a biological macromolecular crystal.
14. Array arranged to perform the method as described in any of embodiments 1-13.
15. Array containing the solutions as described in any of embodiment 11 or 12.

### The Following Embodiments are Also Subject of the Present Invention

1. Method of selecting a soaking solution for soaking the crystal form of a biological macromolecule wherein the soaking solution comprises an organic solvent (os), a compatible solute (cs), and a crystallization solution (crs) and/or water (w); comprising the steps of
   1) preparing a first series of 1 n to xn individual soaking solutions by mixing os, cs, crs and w wherein:
   a) x is at least 2
   b) each soaking solutions of the first series comprises the same organic solvent (os), the same compatible solute (cs), and the same crystallization solution (crs), and
   c) Vos and Vcs are the same in each solution, or, alternatively, Mos/Mcs is the same in each solution, and
   d) V_{crs} and Vw are changed in each solution, and
      in the solution 1 n, V_{crs} is the minimum V_{crs} (V_{crs} min) and the V_{w} is the maximum V_{w} (V_{w} max), and
      in the solution xn, V_{w} is the minimum V_{w} (V_{w} min) and the V_{crs} is the maximum V_{crs}(V_{crs} max), and
      when x>2, in any additional solutions between 1n and xn, the V_{crs} is varied between V_{crs} min and V_{crs} max and, inversely, Vw is varied between V_{w} min and V_{w} max
   2) transferring at least one crystal of the biological macromolecular crystal per compartment into x compartments, each compartment comprising one of the solutions prepared in step 1); or transferring each solution prepared in step 1) into x compartments, each compartment comprising at least one crystal of the biological macromolecular crystal from its crystallization solution.
   3) controlling the crystal in each compartment
   4) selecting the soaking solution(s) which is/are suitable for soaking the crystal; wherein the compatible solute is selected from the group comprising polyols, amino acids, methylamines, and mixtures thereof, and
   wherein the organic solvent is selected from the group comprising liquid carbohydrates, protic or aprotic, of low reactivity, that can serve to solve small molecules and, mixtures thereof, and wherein the crystallization solution crs is obtained from the crystallization process of said crystal or is equivalent thereto in terms of osmotic net pressure, permittivity, solubility of the according biological macromolecule, and effect on its structural conformation, comprising pH and buffer materials, additives, and precipitants.
2. Method of selecting the composition of a soaking solution of embodiment 1, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules, and wherein the amino acids are selected from a group comprising 2-aminoethanesulfonic acid (taurine), 2-aminoethanesulfinic acid (hypotaurine), (2S)-pyrrolidine-2-carboxylic acid (proline), 2-aminoacetic acid (glycine), (6S)-2-methyl-1,4,5,6-tetrahydropyrimidine-6-carboxylic acid (ectoine), (5S,6S)-5-hydroxy-2-methyl-1,4,5,6-tetrahydropyrimidine-6-carboxylic acid (hydroxyectoine), 4-aminobutanoic acid (γ-aminobutyric acid), (2S)-2-aminopentanedioic acid (glutamic acid), β-hydroxy-γ-N-trimethylaminobutyric acid, (2S)-2-amino-3-methylbutanoic acid (valine), (2S,3S)-2-amino-3-methylpentanoic acid (isoleucine), (2S)-2-aminobutanedioic acid (aspartic acid), (2S)-2-aminopropanoic acid (alanine), 2-(methylamino)ethanesulfonic acid (N-methyltaurine), (2S)-2-[[(1R)-1-carboxyethyl]amino]-5-(diaminomethylideneamino)pentanoic acid (Octopin) and wherein the methylamines are selected from a group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), 2-hydroxyethyl(trimethyl)azanium (choline), 2-[carbamimidoyl(methyl)amino]acetic acid (creatine), L-alpha-Glycerylphosphorylcholin, (R)-(3-Carboxy-2-hydroxypropyl)-N,N,N-trimethylammoniumhydroxid (carnitine).
3. Method of selecting the composition of a soaking solution of embodiment 1 or 2, wherein the compatible solute is selected from the group comprising polyols, amino acids, methylamines or mixtures thereof, wherein the polyols are selected from a group comprising poly(oxyethylene), polyhydric alcohols, monosaccharide, disaccharide, trisaccharide, sugar alcohols, cyclitols and derivatives of former molecules wherein the group of poly(oxyethylene) is selected from a group comprising monodisperse or polydisperse poly(oxyethylene) of molecular weights ranging between 20-2000000 g/mol and includes but is not limited to for example polyethylene glycol 400, polyethylene glycol 600 and wherein the polyhydric alcohol is selected from the group comprising propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol) and wherein the monosaccharide is selected from the group comprising (2R,3S,4R,5R)-2,3,4,5,6-Pentahydroxyhexanal (glucose), (3S,4R,5R)-1,3,4,5,6-Pentahydroxyhexan-2-one (fructose)), (2S,3R,4S,5R,6R)-2-(2,3-dihydroxypropoxy)-6-(hydroxymethyl)oxane-3,4,5-triol (isofloridoside) and wherein the group of disaccharides is selected from the group comprising β-D-Fructofuranosyl α-D-glucopyranoside (sucrose), (2R,3S,4S,5R,6R)-2-(Hydroxymethyl)-6-[(2R,3R,4S,5S,6R)-3,4, 5-trihydroxy-6-(hydroxymethyl)oxan-2-yl]oxyoxane-3,4,5-triol (trehalose) and wherein the group of trisaccharides is selected from the group comprising (2R,3R,4S,5S,6R)-2-[(2S,3S,4S,5R)-3,4-dihydroxy-2,5-bis(hydroxymethyl)oxolan-2-yl]oxy-6-[[(2S,3R,4S,5R,6R)-3,4,5-trihydroxy-6-(hydroxymethyl)oxan-2-yl]oxymethyl]oxane-3,4,5-triol (raffinose) and wherein the group of sugar alcohols comprises (2R,3R,4R,5R)-Hexane-1,2,3,4,5,6-hexol (mannitol), (2S,3R,4R,5R)-Hexane-1,2,3,4,5,6-hexol (sorbitol), (1R,2S,3r,4R,5S,6s)-cyclohexane-1,2,3,4,5,6-hexol (inositol), (2R,3R,4S)-Pentane-1,2,3,4,5-pentol (xylitol), (2R,3s,4S)-Pentane-1,2,3,4,5-pentol (adonitol), (2R,3S)-Butane-1,2,3,4-tetrol (erythritol), (2R,4R)-Pentane-1,2,3,4,5-pentol (arabinitol) and (2R,3S,4R,5S)-hexane-1,2,3,4,5,6-hexol (galactitol)) and wherein the group of cyclitols comprises (1S,2S,4S,5R)-6-methoxycyclohexane-1,2,3,4,5-pentol (pinitol) and (2S,3R,4S,5R,6R)-2-[(1,3-dihydroxypropan-2-yl)oxy]-6-(hydroxymethyl)oxane-3,4,5-triol (fluoridoside) and wherein the group of derivatives of former molecules comprises mannose derivatives as fiorin ((2R)-O2-(β-D-mannopyranosyl)glyceric acid) and fiorin-A (mannosylglyceramide), inositol derivatives (preferably di-myoinositol-1,1′-phosphate), glycerol derivatives (preferably cyclic 2,3-diphosphoglycerate, alpha-diglycerol phosphate) and polymers of former molecules like starch, fructan, cellulose.
4. Method of selecting the composition of a soaking solution according to any of embodiments 1 to 3, wherein the organic solvent is a straight or branched chain monohydric aliphatic alcohol, dihydric aliphatic alcohol, monoalkyl ether of an aliphatic dihydric alcohol, ether of an aliphatic monohydric alcohol, cyclic non-aromatic ethers, (cyclic) enol-ethers, dialkyl ketones, benzyl alcohols, phenyl ethyl alcohols, (alkylated) sulfoxides and according phosphoderivatives, esters derived from acetic acid, (alkylated) formamides or mixtures thereof.
5. Method of selecting the composition of a soaking solution of embodiment 1 to 4, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the pH and buffer materials are selected from a group comprising acetate-, TRIS, HEPES-, cacodylate-, 1H-imidazole (imidazole) and citrate-buffer and wherein the precipitant is selected from a group comprising volatile organic solvents, salts, polymers or non-volatile organic solvents, and wherein the additives are selected from a group comprising physiologically or biochemically relevant small molecules, chemical protectants, solubilizing agents and detergents, compounds that reduce twinning, osmolytes, co-solvents and cosmotropes, classes of compounds that cross-link carboxyl and amino groups on the protein surface, classes of compounds that form electrostatic, covalent or hydrogen-bonding arrangements intended to stabilize the biological macromolecular crystals and classes of materials or compounds that serve to enhance nucleation and providing unique surfaces.
6. Method of selecting the composition of a soaking solution of embodiment 1 to 5, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the precipitant is selected from a group comprising volatile organic solvents, salts, polymers or non-volatile organic solvents and wherein the volatile organic solvents is selected from a group comprising ethanol, propan-1-o, propan-2-ol, 2-propanol, 1,4-dioxane (dioxane), propan-2-one (acetone), 2-methylpropan-1-ol (isobutanol), butan-1-ol (n-butanol), 2-methylpropan-2-ol (tert-butanol), acetonitrile, methylsulfinylmethane (dimethyl sulfoxide), oxolan-2-one (gamma-butyrolactone), 4-methyloxetan-2-one (beta-butyrolactone)) and wherein the salt is selected from the group comprising disodium disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), Ammonium 2-hydroxypropane-1,2,3-tricarboxylate (ammonium citrate), sodium ethanoate (sodium acetate), ammonium ethanoate (ammonium acetate), lithium sulfate, lithium chloride, sodium chloride, potassium chloride, sodium methanoate (sodium formate), monophosphate from sodium and potassium, di- and polyphospahtes from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium diformate, magnesium dichloride, magnesium sulfate, calcium chloride, disodium butanedioate (sodium succinate), cadmium(II) sulfate (cadmium sulfate), Disodium propanedioate (sodium malonate), magnesium; diacetate (magnesium acetate), zinc; diacetate (zinc acetate), calcium; diacetate (calcium acetate) and wherein the polymers is selected from a group comprising polyethylene glycols (preferably PEG400, PEG1000, PEG3350, PEG4000, PEG6000, PEG8000, PEG20000), Jeffamine T, Jeffamine M, 2-methoxyethanol (polyethylene glycol monomethyl ester), 2-hydroxyethyl octadecanoate (polyethylene glycol monostearate) and polyeneamine and wherein the non-volatile organic solvents is selected from a group comprising 2-methyl-2,4-pentanediol, hexane-2,5-diol (2,5-Hexanediol), propane-1,3-diol (1,3-propanediol), polyethylene glycol (for example 400), Jeffamine (for example 400).
7. Method of selecting the composition of a soaking solution of embodiment 1 to 6, wherein the crystallization solution crs is obtained from the crystallization process of said crystal or is equivalent thereto in terms of osmotic net pressure, permittivity, solubility of the according biological macromolecule and effect on its structural conformation and comprises pH and buffer materials, additives, and precipitants or mixtures thereof and wherein the group of additives is selected from a group comprising physiologically or biochemically relevant small molecules, chemical protectants, solubilizing agents and detergents, compounds that reduce twinning, osmolytes, co-solvents and cosmotropes, classes of compounds that cross-link carboxyl and amino groups on the protein surface, classes of compounds that form electrostatic, covalent or hydrogen-bonding arrangements intended to stabilize the biological macromolecular crystals and classes of materials or compounds that serve to enhance nucleation and providing unique surfaces wherein the protein physiologically or biochemically relevant small molecule is selected from a group comprising 5-[(3aS,4S,6aR)-2-oxo-1,3,3a,4,6,6a-hexahydrothieno[3,4-d]imidazol-4-yl]pentanoic acid biotin, [[(2R,3S,4R,5R)-5-(6-aminopurin-9-yl)-3,4-dihydroxyoxolan-2-yl]methoxy-hydroxyphosphoryl] phosphono hydrogen phosphate (adenosine triphosphate), [(2R,3S,4R,5R)-5-(6-aminopurin-9-yl)-3,4-dihydroxyoxolan-2-yl]methyl phosphono hydrogen phosphate (adenosine diphosphate), [(2R,3S,4R,5R)-5-(6-aminopurin-9-yl)-3,4-dihydroxyoxolan-2-yl]methyl dihydrogen phosphate (adenosine monophosphate), 3-[18-(2-carboxylatoethyl)-7,12-bis(ethenyl)-3,8,13,17-tetramethylporphyrin-21,23-diid-2-yl]propanoate; iron (Fe-Protoporphyrin IX or heme b) and wherein the chemical protectants are selected from a group comprising 2-sulfanylethanol (p-mercaptoethanol or BME) and (2S,3S)-1,4-bis(sulfanyl)butane-2,3-diol (dithiothreitol or DTT), 3-[bis(2-carboxyethyl)phosphanyl]propanoic acid; hydrochloride (TCEP), heavy-metal ion scavengers as 2-[2-[bis(carboxymethyl)amino]ethyl-(carboxymethyl)amino]acetic acid (Ethylenediaminetetraacetic acid or EDTA) and 2-[2-[2-[2-[bis(carboxymethyl)amino]ethoxy]ethoxy]ethyl-(carboxymethyl)amino]acetic acid (EGTA), compounds to prevent microbial infections as sodium; azide (sodium azide), phenol, 1,1,1-trichloro-2-methylpropan-2-ol (chlorobutanol) and wherein the solubilizing agents and detergents are selected from a group comprising quaternary ammonium salts, sulfobetains, chaotropes as urea, as well as surfactants and detergent molecules as (2R,3S,4S,5R,6R)-2-(hydroxymethyl)-6-octoxyoxane-3,4,5-triol (n-octyl-beta-d-glucoside), octyl-polyoxyethylene (octyl-POE), N,N-dimethyldodecan-1-amine oxide (lauryldimethylamine oxide), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (n-dodecyl-β-D-maltoside), sodium; 1,4-bis(2-ethylhexoxy)-1,4-dioxobutane-2-sulfonate (bis(2-ethylhexyl)sulfosuccinate), dodecylazanium; chloride (dodecylammonium chloride), polyethoxylated fatty acids and wherein the compounds that are meant to reduce twinning are selected from the group comprising ethanol, methylsulfinylmethane (dimethyl sulfoxide or DMSO), propan-2-one (acetone), 1,4-dioxane (dioxane), butan-1-ol (butanol), 2-methylpentane-2,4-diol (2-Methyl-2,4-pentanediol or MPD) and wherein the osmolyte, co-solvent and cosmotrope is slected from the group comprising (2R,3R,4S,5S,6R)-2-[(2S,3S,4S,5R)-3,4-dihydroxy-2,5-bis(hydroxymethyl)oxolan-2-yl]oxy-6-(hydroxymethyl)oxane-3,4,5-triol (sucrose), (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) and other sugars (2S)-pyrrolidine-2-carboxylic acid (proline), N,N-dimethylmethanamine oxide (trimethylamine N-oxide or TMAO), 2-aminoacetic acid (glycine), 2-(trimethylazaniumyl)acetate (betaine), 2-aminoethanesulfonic acid (taurine), 2-(methylamino)acetic acid (sarcosine) and wherein the class of compounds that cross-link carboxyl and amino groups on the surface of proteins is selected from the group comprising diamino-containing or dicarboxylic acid-containing molecules or aliphatic moieties of various length carrying charged groups and wherein the class of compounds that form electrostatic, covalent or hydrogen-bonding arrangements intended to stabilize crystals by intermolecular cross-linking between proteins in a crystal is pentanedial (glutaraldehyde) and wherein the class of materials or compounds that serve to enhance nucleation and providing unique surfaces is selected from the group comprising PEG, Jeffamine, gels, gels as used in cubic lipidic phase crystallization and surfaces which promote epitaxy and heterogeneous nucleation, commercially available nucleants like Crystallophore No. 1, Naomi's nucleant, JBS Magical Triangle, Anderson-Evans polyoxotungstate (Na6[TeW6024]×22 H2O) or components of buffer solutions that influence the macromolecule's solubility and aggregation behavior and as a consequence their nucleation and growth behavior as 2-aminoacetic acid (glycine), 3-[4-(3-sulfopropyl)piperazin-1-yl]propane-1-sulfonic acid (PIPPS), 2-[bis(2-hydroxyethyl)amino]-2-(hydroxymethyl)propane-1,3-diol (Bis-Tris), 2-morpholin-4-ylethanesulfonic acid (MES), 2-[(2-amino-2-oxoethyl)-(carboxymethyl)amino]acetic acid (ADA), 2-[3-[[1,3-dihydroxy-2-(hydroxymethyl)propan-2-yl]amino]propylamino]-2-(hydroxymethyl)propane-1,3-diol (Bis-Tris Propane), 3-morpholin-4-ylpropane-1-sulfonic acid (MOPS), 2-[4-(2-hydroxyethyl)piperazin-1-yl]ethanesulfonic acid (HEPES), 2-amino-2-(hydroxymethyl)propane-1,3-diol (TRIS), 3-[4-(2-hydroxyethyl)piperazin-1-yl]propane-1-sulfonic acid (EPPS), 1H-imidazole (imidazole), 2-[bis(2-hydroxyethyl)amino]acetic acid (bicine), 2-(cyclohexylamino)ethanesulfonic acid (CHES), 3-(cyclohexylamino)propane-1-sulfonic acid (CAPS), (2R,3S,4S,5R)-2-(hydroxymethyl)-6-octoxyoxane-3,4,5-triol (octyl glucopyranoside), (2R,3R,4S,5S,6R)-2-[(2R,3S,4R,5R,6R)-6-dodecoxy-4,5-dihydroxy-2-(hydroxymethyl)oxan-3-yl]oxy-6-(hydroxymethyl)oxane-3,4,5-triol (dodecyl maltoside), (2S,3S)-1,4-bis(sulfanyl)butane-2,3-diol (DTT), 3-[bis(2-carboxyethyl)phosphanyl]propanoic acid (TCEP).
8. Method of selecting the composition of a soaking solution according to any of embodiments 1 to 7, wherein said soaking solution comprises a crystallization solution (crs) or an equivalent aqueous salt solution thereto and an organic solvent (os) and a compatible solute (cs),
   wherein said compatible solute (cs) is a polyol a or a mixture thereof, and said organic solvent (os) is a liquid carbohydrate protic or aprotic, of low reactivity, or a mixture thereof, that can serve to solve small molecules,
   more preferably, said compatible solute (cs) is a poly(oxyethylene) and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute (cs) is selected from the group comprising the poly(oxyethylene)s polyethylene glycol 400, polyethylene glycol 600 or a mixture thereof and said organic solvent (os) is selected from the group comprising dimethyl sulfoxide, 1,4-dioxane, dimethylformamide, methanol, ethanol, or a mixture thereof and
   wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs: Vos);
   and wherein the ratio of volume crystallization solution or equivalent aqueous solution Vcrs to the combined volumes of organic solvent Vos and compatible solute Vcs is 1:1000000 to 1000000:1, preferably between 99:1 to 1:99 and most preferably between 85:15 to 1:1 (Vcrs:(Vos; Vcs)).
9. Method of selecting the composition of a soaking solution according to any of embodiments 1 to 7, wherein said soaking solution comprises a crystallization solution (crs) or an equivalent aqueous salt solution thereto and an organic solvent (os) and a compatible solute (cs),
   wherein said compatible solute (cs) is a polyol or a mixture thereof, and said organic solvent is a (alkylated) sulfoxide, cyclic non-aromatic ether, straight or branched chain monohydric aliphatic alcohol, alkylated formamide, or a mixture thereof,
   more preferably, said compatible solute (cs) is a polyhydric alcohol or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute (cs) is selected from the group comprising the polyhydric alcohols propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol) or a mixture thereof, and said organic solvent (os) is selected from the group comprising dimethyl sulfoxide, 1,4-dioxane, dimethylformamide, methanol, ethanol, or a mixture thereof and
   wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs: Vos);
   and wherein the ratio of volume crystallization solution or equivalent aqueous solution Vcrs to the combined volumes of organic solvent Vos and compatible solute Vcs is 1:1000000 to 1000000:1, preferably between 99:1 to 1:99 and most preferably between 85:15 to 1:1 (Vcrs:(Vos; Vcs)).
10. Method of selecting the composition of a soaking solution according to any of embodiments 1 to 7, wherein said soaking solution comprises a crystallization solution (crs) or an equivalent aqueous salt solution thereto and an organic solvent (os) and a compatible solute (cs),
   wherein said compatible solute (cs) is a polyol a or a mixture thereof, and said organic solvent (os) is a liquid carbohydrate protic or aprotic, of low reactivity, or a mixture thereof, that can serve to solve small molecules,
   more preferably said compatible solute (cs) is a mono-, di-, tri, oligo- or polysaccharid or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
   most preferably, the compatible solute cs is selected from the group comprising the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from the group comprising dimethyl sulfoxide, 1,4-dioxane, dimethylformamide, methanol, ethanol, or a mixture thereof and
   wherein the molar ratio of compatible solute to organic solvent is the same and the molar ratio is 1:10000 to 10000:1, preferably between 100:1 to 1:100 and more preferably between 10:1 to 1:10,
   and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos; Vcs)).
11. Method of selecting the composition of a soaking solution according to any of embodiments 1 to 7, wherein said soaking solution comprises a crystallization solution (crs) or an equivalent aqueous salt solution thereto and an organic solvent (os) and a compatible solute (cs),
   wherein said compatible solute (cs) is a L- or D amino acid, or a mixture thereof, and the organic solvent (os) is liquid carbohydrate protic or aprotic, of low reactivity, or a mixture thereof, that can serve to solve small molecules,
   more preferably said compatible solute (cs) is a L-amino acid, or a mixture thereof, and said organic solvent is a (alkylated) sulfoxide, cyclic non-aromatic ether, straight or branched chain monohydric aliphatic alcohol, alkylated formamide, or a mixture thereof,
   most preferably, said compatible solute (cs) is selected from the group comprising the L-amino acids (2S)-pyrrolidine-2-carboxylic acid (proline), 2-aminoacetic acid (glycine), (6S)-2-methyl-1,4,5,6-tetrahydropyrimidine-6-carboxylic acid (ectoine) or (5S,6S)-5-hydroxy-2-methyl-1,4,5,6-tetrahydropyrimidine-6-carboxylic acid (hydroxyectoine), or a mixture thereof, and said organic solvent (os) is selected from the group comprising dimethyl sulfoxide, 1,4-dioxane, dimethylformamide, methanol, ethanol, or a mixture thereof and
   wherein the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10,
   and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos; Vcs)).
12. Method of selecting the composition of a soaking solution according to any of embodiments 1 to 7, wherein said soaking solution comprises a crystallization solution (crs) or an equivalent aqueous salt solution thereto and an organic solvent (os) and a compatible solute (cs),
   wherein said compatible solute (cs) is a methylamine or a mixture thereof and said organic solvent (os) is liquid carbohydrate protic or aprotic, of low reactivity, or a mixture thereof that can serve to solve small molecules,
   more preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
   most preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from the group comprising dimethyl sulfoxide, 1,4-dioxane, dimethylformamide, methanol, ethanol, or a mixture thereof and
   wherein the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10,
   and wherein the ratio of volume crystallization solution or equivalent aqueous solution Vcrs to the combined volumes of organic solvent Vos and compatible solute Vcs is 1:1000000 to 1000000:1, preferably between 100:1 to 1:100 and most preferably between 85:15 to 1:1 (Vcrs:(Vos; Vcs)).
13. Method of selecting the composition of a soaking solution according to any of embodiments 1 to 8, wherein said soaking solution comprises a crystallization solution (crs) or an equivalent aqueous salt solution thereto and an organic solvent (os) and a compatible solute (cs),
   wherein said compatible solute (cs) is polyethylene glycol 400 and said organic solvent (os) is dimethyl sulfoxide,
   wherein the ratio of volume Vcs polyethylene glycol 400 to volume Vos dimethyl sulfoxide is the same and the volume ratio is 1:1000000 to 1000000:1, preferably between 8:1 to 1:8 and more preferably between 3:1 to 1:1 (Vcs: Vos);
   and wherein the ratio of volume crystallization solution or equivalent aqueous solution Vcrs to the combined volumes of the organic solvent (os) dimethyl sulfoxide and the compatible solute (cs) polyethylene glycol 400 is 1:1000000 to 1000000:1, preferably between 99:1 to 1:99 and most preferably between 85:15 to 1:1 (Vcrs:(Vos:Vcs)).
14. Method of selecting the composition of a soaking solution according to any of embodiments 1 to 7 and 9, wherein said soaking solution comprises a crystallization solution (crs) or an equivalent aqueous solution thereto and an organic solvent (os) and a compatible solute (cs),
   wherein said compatible solute (cs) is ethane-1,2-diol (ethylene glycol) and said organic solvent (os) is dimethyl sulfoxide,
   wherein the ratio of volume Vcs ethane-1,2-diol (ethylene glycol) to volume Vos dimethyl sulfoxide is the same and the volume ratio is 1:1000000 to 1000000:1, preferably between 8:1 to 1:8 and more preferably between 3:1 to 1:1 (Vcs: Vos);
   and wherein the ratio of volume crystallization solution or equivalent aqueous solution Vcrs to the combined volumes of the organic solvent (os) dimethyl sulfoxide and the compatible solute (cs) ethane-1,2-diol (ethylene glycol) is 1:1000000 to 1000000:1, preferably between 99:1 to 1:99 and most preferably between 85:15 to 1:1 (Vcrs (Vos:Vcs)).
15. Method of selecting the composition of a soaking solution according to any of embodiments 1 to 7 and 9, wherein said soaking solution comprises a crystallization solution (crs) or an equivalent aqueous solution thereto and an organic solvent (os) and a compatible solute (cs),
   wherein said compatible solute (cs) is propane-1,2,3-triol (glycerol) and said organic solvent (os) is dimethyl sulfoxide,
   wherein the ratio of volume Vcs propane-1,2,3-triol (glycerol) to volume Vos dimethyl sulfoxide is the same and the volume ratio is 1:1000000 to 1000000:1, preferably between 8:1 to 1:8 and more preferably between 3:1 to 1:1 (Vcs: Vos);
   and wherein the ratio of volume crystallization solution or equivalent aqueous solution Vcrs to the combined volumes of the organic solvent (os) dimethyl sulfoxide and the compatible solute (cs) propane-1,2,3-triol (glycerol) is 1:1000000 to 1000000:1, preferably between 99:1 to 1:99 and most preferably between 85:15 to 1:1 (Vcrs:(Vos:Vcs)).
16. Method of selecting the composition of a soaking solution according to any of embodiments 1 to 7 and 9, wherein said soaking solution comprises a crystallization solution (crs) or an equivalent aqueous solution thereto and an organic solvent (os) and a compatible solute (cs),
   wherein said compatible solute (cs) is 2-methylpentane-2,4-diol (MPD) and said organic solvent (os) is dimethyl sulfoxide,
   wherein the ratio of volume Vcs 2-methylpentane-2,4-diol (MPD) to volume Vos dimethyl sulfoxide is the same and the volume ratio is 1:1000000 to 1000000:1, preferably between 8:1 to 1:8 and more preferably between 3:1 to 1:1 (Vcs:Vos);
   and wherein the ratio of volume crystallization solution or equivalent aqueous solution V_{crs} to the combined volumes of the organic solvent (os) dimethyl sulfoxide and the compatible solute (cs) 2-methylpentane-2,4-diol (MPD) is 1:1000000 to 1000000:1, preferably between 99:1 to 1:99 and most preferably between 85:15 to 1:1 (V_{crs} (Vos:Vcs)).
17. Method of selecting the composition of a soaking solution of any of embodiment 1-16 further comprising the step of
   1) preparing m series of 1 n to yn individual soaking solutions by mixing os, cs, crs and w wherein m is at least 1 and wherein in each of the m series
   a) y is at least 2
   b) os and crs are the same as in the first series, and
   c) Vos and Vcs are the same as in the first series, or, alternatively, Mos/Mcs is the same in the first series,
   d) the cs is changed and is different from the cs of the first series, and
   wherein in each individual solutions of each of the m series
   e) Vos and Vcs are conserved, or, alternatively, Mos/Mcs is conserved, and
   f) V_{crs} and Vw are changed in each solution, and
      in the solution 1 n, V_{crs} is the minimum V_{crs} (V_{crs} min) and the V_{w} is the maximum V_{w} (V_{w} max), and
      in the solution yn, V_{w} is the minimum V_{w} (V_{w} min) and the V_{crs} is the maximum V_{crs}(V_{crs} max), and
      when y>2, in any additional solutions between 1n and yn, the V_{crs} is varied between V_{crs} min and V_{crs} max and, inversely, Vw is varied between V_{w} min and V_{w} max
   2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.
18. Method of selecting the composition of a soaking solution of any of the embodiments 1 to 17 wherein an equivalent aqueous salt solution different from the crystallization solution is used, and wherein the salt selected from the group comprising disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), triazanium; 2-hydroxypropane-1,2,3-tricarboxylate (ammonium citrate), sodium; acetate (sodium ethanoate), azanium; acetate (ammonium acetate), dilithium; sulfate, lithium; chloride, lithium; acetate, lithium; formate, lithium; nitrate, sodium; nitrate, sodium; chloride, potassium; chloride, sodium; formate, monophosphate from sodium and potassium, di- and polyphosphates from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate)), magnesium; diformate, magnesium; dichloride, magnesium; sulfate, calcium; dichloride; dihydrate, disodium; butanedioate (sodium succinate), cadmium(II) sulfate (cadmium sulfate), disodium; propanedioate (sodium malonate), magnesium; diacetate (magnesium acetate), zinc; diacetate (zinc acetate), calcium; diacetate (calcium acetate) and wherein the salt for the equivalent solution is preferably selected from the group comprising lithium; acetate, lithium; chloride, lithium; formate, lithium; nitrate, dilithium; sulfate, sodium; chloride, potassium; chloride, magnesium; dichloride, diazanium; sulfate (ammonium sulfate), sodium; acetate (sodium ethanoate), sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium; diformate, sodium; nitrate.
19. Method of selecting the composition of a soaking solution of embodiment 1-18 wherein the salt is sodium; chloride, potassium; chloride, magnesium; diacetate (magnesium acetate) and dilithium; sulfate.
20. Method of selecting the composition of a soaking solution of any of the embodiments 1-19 wherein the percentage of the total volume (VT) of each solution of the first series is VT=Vcrs+Vw+Vos+Vcs=100%.
21. Method of selecting the composition of a soaking solution of any of the embodiments 1-20 wherein the step of controlling the crystal is performed by X-Ray-diffraction neutron diffraction, absorption spectroscopy or reflectance spectroscopy in ultraviolet and visible spectral regions, infrared spectroscopy and/or visual analysis.
22. Method of selecting the composition of a soaking solution of any of the embodiments 1-21 wherein the os contains small molecules including small fragments and small probes to be analyzed, which diffuse into the biological macromolecular crystal.
23. Method of small molecule screening for a biological macromolecular crystal wherein the screening is performed in a soaking solution selected according to the method of embodiment 22.
24. Soaking solution obtainable by the method of selecting the composition of a soaking solution as described in any of the embodiments 1-22.
25. Soaking solution as described in embodiment 24 wherein Vos is at least 0.5%.
26. Use of the soaking solution as described in any of embodiment 24 or 25 for small molecule screening for a biological macromolecular crystal.
27. Array arranged to perform the method as described in any of embodiments 1-23, wherein said array comprises a first dimension of at least two individual soaking solutions (1n to xn) and a second dimension of at least two individual soaking solutions (1m to ym) wherein each of said soaking solutions is located in a separated compartment of said array, and wherein each of said soaking solution comprises an organic solvent (os), a compatible solute (cs), a crystallization solution (crs) and water, and wherein the ratio of volume compatible solute Vcs to volume organic solvent Vos is the same within a series of soaking solutions in said first dimension, or, alternatively, the molar ratio of organic solvent (os) to compatible solute (cs) Mos/Mcs is the same within a series of soaking solutions in said first dimension, and wherein the individual soaking solutions of said first dimension comprise the same organic solvent and the same compatible solute within a series of said soaking solutions in the first dimension, respectively, and wherein the ratio of the volume of water Vw and the volume of the crystallization solution Vcrs is varied over the individual soaking solutions of the series in the first dimension and wherein one of the individual soaking solutions of the series in the first dimension has a minimal or zero Vcrs and a maximum Vw and another individual soaking solution of said first dimension has a minimal or zero Vw and a maximum Vcrs and wherein the other individual soaking solutions of the series in said first dimension take values of Vcrs and Vw in between the two before-mentioned values.
28. Array according to embodiment 27, wherein 1 n to xn defines the numbers of individual soaking solution compositions in a range of between 1 (1 n) to 1×10⁶ (xn) and wherein the numbers comprise but are not limited to 1×10⁶ or 1×10⁵ 1×10⁴ or 768 or 384 or 96 or 48 or 24, however, the set-up of the array determines the numbers of individual soaking solutions.
29. Array according to embodiments 27 and 28 wherein the array comprises a second dimension (dll) of a series of individual soaking solutions for a biological macromolecular crystal wherein the compatible solute is varied over said second dimension and the ratio of volume compatible solute Vcs to volume organic solvent Vos or, alternatively, the molar ratio of organic solvent (os) to compatible solute (cs) Mos/Mcs, may be different dependent on the compatible solute used in the series of soaking solutions in said second dimension.
30. Array according to any of embodiments 27 to 29, wherein in each of said individual soaking solutions in a series the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs: Vos).
31. Array according to any of embodiments 27 to 30, wherein in each of said individual soaking solutions in a series the molar ratio of organic solvent (os) to compatible solute (cs) Mos/Mcs is the same and the molar ratio Mos/Mcs is 1:10000 to 10000:1, preferably between 100:1 to 1:100 and more preferably between 10:1 to 1:10.
32. Use of an Array according any of embodiments 27-31 for obtaining the solutions as described in any of embodiments 22 or 23.

### FIGURE DESCRIPTION

FIG. 1: 1A is a picture of a screening plate used for soaking selection; FIG. 1B shows the details of one of the wells of a 24 well plate and on FIG. 1C a schematic representation of the well during a soaking experiment is depicted.

FIG. 2 shows a preparation scheme according to the method of the invention for selection of soaking solutions comprising CS: compatible solute; OS—organic solvent; CB (crs)—crystallization buffer (solution); W—water. The lines (solute A-D) represent the first dimension and the columns (1-6) the second dimension. Alternatively, the lines (solute A-D) can represent the second dimension and the columns (1-6) the first dimension

FIG. 3 shows an example of a scheme according to the method of the invention for selection of soaking solutions comprising for fragment screening on PKA. PEG400—polyethylene glycol 400; MPD—2-methylpentane-2,4-diol; EG—ethane-1,2-diol (ethylene glycol); Gly-propane-1,2,3-triol (glycerol); OS—organic solvent=DMSO; CB (crs)—crystallization buffer (solution); W—water.

FIG. 4 shows photographs of crystals after 24 h in the soaking solutions of FIG. 3

FIG. 5: shows an example of the visual difference between suitable crystal (A) and crystals suffering from a severe quality loss (B) due to detrimental soaking conditions/solution

FIG. 6 shows a template for the visual inspection of crystals under i.e. a microscope.

FIG. 7: shows the criteria used for visual inspection and to control and select the solutions having minimal impact on the crystal

FIG. 8: shows the results of the visual inspection of PKA crystals after 24 h in the tested soaking solutions of FIG. 3. Each series was prepared with different composite solutes: PEG400; MPD; EG—ethane-1,2-diol (ethylene glycol) or (glycerol); S&C stands for surface and cracks; EDG stands for edges; COL stands for colour; SMS stands for smooth surface; SME stands for smooth edges; Y− stands for yes but little; Y+ stands for yes many; LCR stands for longitudinal cracks; CCC stands for criss-cross cracks; ROS stands for rough surface; FRE stands for frayed edges; N stands for no; DIS stands for dissolving; X stands for no data.

FIG. 9 shows an image of a diffraction pattern after an X-Ray experiment.

FIG. 10 shows a template for the X-Ray inspection of crystals.

FIG. 11: shows the criteria used for X-Ray inspection and to control and select the solutions having minimal impact on the crystal.

FIG. 12 shows the results of x-ray examination of crystals after 24 h in the soaking solutions of FIG. 3. RES stands for resolution; MOS stands for mosaicity; TWI stands for twinning; ICE stands for ice rings.

FIG. 13 highlights the most suitable soaking conditions on the scheme thus providing a range/scope of suitable soaking parameters. The star indicates one of the soaking conditions which was chosen for the subsequent fragment screening.

FIG. 14 shows a template scheme for fragment screening.

FIG. 15 shows the possible scheme using one of the compositions highlighted in FIG. 13 (PEG 400, 3rd column) for the fragment screening on PKA.

FIG. 16 shows the possible scheme using one (with the star) of the compositions highlighted in FIG. 13 (PEG 400, 1st column) for the fragment screening on PKA. The ending “Frag”+number indicates individual fragments.

FIG. 17 shows an example of a potential workflow representing the different step of the processes of the invention performed in a soaking selection experiment with subsequent fragment screening.

FIG. 18 shows the distribution of hits over the example plate illustrated in FIG. 16. The plate comprises the first 24 fragments out of 87 fragments. Five fragments form this plate turned out to be PKA-binders (see boxes A1, A4, B6, D3 and D5). RES and MOS indicate the data set's quality in terms of resolution (RES) and mosaicity (MOS).

FIG. 19 shows a 3D-model of PKA representing the protein and an overlay of all identified binders distributed over different locations on the protein. The magnification in the circle shows the crowd of ATP-pocket binders in more detail.

FIG. 20 shows the excerpt of a reconstructed electron density.

FIG. 21 shows the same as FIG. 20 but completed with the protein's amino acid sequence and structurally fixed water molecules (spheres).

FIG. 22: shows the same as FIG. 20 but with highlighted (ellipse) unassigned electron density that is representative for a small molecule fragment-hit.

FIG. 23 shows the same as FIG. 20 but completed with the representation of the respective small molecule fragment in the formerly unassigned electron density highlighted in FIG. 19.

FIG. 24 shows the excerpt of the structure elucidating the binding mode in terms of hydrogen bonds (dotted lines; numbers represent the length of the hydrogen bonds) of the small molecule fragment to the amino acid side chains of the protein and to structurally fixed water (dot).

### EXAMPLES

### Example 1: Screening of 87 Fragments Using Crystal of the Protein Kinase a (PKA)

Crystallization of PKA

A PKA solution was concentrated to 8-10 mg/mL by filtration centrifugation. At the same time a buffer exchange was conducted against 100 mM Mes-Bis-Tris buffer (pH 6.9) containing 1 mM DTT, 0.1 mM EDTA (ethylenediaminetetraacetic acid), and 75 mM LiCl. Afterwards the solution was sterile-filtrated. Of this solution, a volume of 72 μL was mixed with 8 μL of 1 M Mes-Bis-Tris buffer (pH 6.9), and 2 μL of 10 mM Mega 8 solution and centrifuged 15 min. Crystallization was performed by the vapor-diffusion method at 4° C. using 3 μl-sitting drops of the master-mix against 400 μL of methanol/water solutions with methanol concentrations of 14-23% (v/v).

Selection of the Suitable Soaking Solution

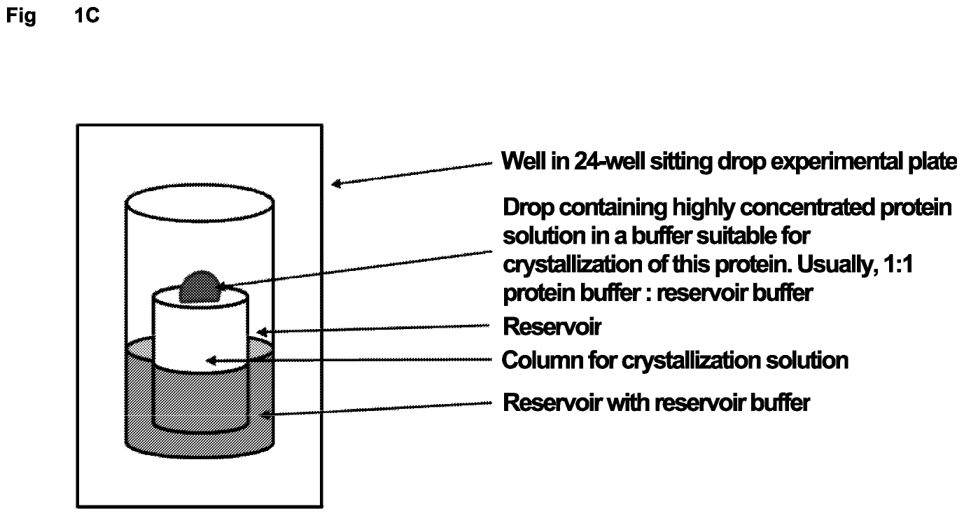

The crystals were harvested via a cryo-loop and transferred to the wells of an experimental plate that was prepared and distributed in two dimensions according to the method of the invention (FIG. 3).

Preparation of a first series of 6 individual soaking solutions by mixing os, w and crs and cs. As organic solvent DMSO was used in a concentration of 10% (Vos). Compatible solutes are polyethylene glycol 400 (PEG 400) and 2-methylpentane-2,4-diol (MPD) at a concentration of 25%, ethane-1,2-diol (ethylene glycol; EG) or propane-1,2,3-triol (glycerol; Gly) at concentrations of 10%. As third component dilutions in water (W) of the crystallization buffer (crs named CB in FIG. 3) were used according to the method of the invention.

The series of line A which was prepared with PEG400 as cs can be designated as the first series according to the invention. Each soaking solutions of the first series comprises the same organic solvent (os) (DMSO) and the same compatible solute (cs) and the Vos 10% (1 μl) and Vcs 25% (2.5 μl) remains the same in all solutions of the series. However, the volume/proportion of Vcrs (CB) and Vw are changed in each solution of the first series, and one of the solution is composed of Vwmin is 0% (0 μl) and Vcrsmax is 65% (6.5 μl) (see column 1), and another solution is composed of Vcrsmin is 0% (0 μl) and the Vwmax is 65% (6.5 μl) (see column 6) and in the other solutions (see columns 2-5) the Vw is varied between Vwmin and Vwmax and, inversely, Vcrs is varied between Vcrsmin and Vcrsmax. In this example, Vcrs is incrementally varied between Vcrsmin and Vcrsmax and, inversely, Vw is incrementally varied between Vwmin and Vwmax in any additional solutions (see column 2-5 of line PEG400) and in any additional solutions, the increment of Vw between Vwmin and Vwmax and of Vcrs between Vcrsmin and Vcrsmax remains the same, 13% (1.3 μl) between each solution of the first series.

The solutions were distributed in the wells of a line (line A on FIG. 3) of an array thus constituting a first dimension. The solutions were preferably distributed following an order from the lowest Vw to the highest Vw but the order of the distribution in the array can be random.

Then or simultaneously, the following step were performed as follows Preparation of an additional series of 6 individual soaking solutions by mixing os, w, crs (CB) and cs. In this example of FIG. 3, the number of individual soaking solutions 6 is conserved and is the same as the number of individual soaking solutions of the first series. In the additional series (line B), os is the same as in the first series (DMSO), but the cs is changed (line B: BMPD). Within the additional MPD series, each soaking solutions of the first series comprises the same organic solvent (os) (DMSO) and the same compatible solute (cs) (MPD) and the Vos and Vcs remains the same in all solutions of the series. However, the volume/proportion of Vcrs (CB) and Vw are changed in each solution of the series, and one of the solution is composed of Vwmin is 0% (0 μl) and Vcrsmax is 65% (6.5 μl) (see column 1), and another solution is composed of Vcrsmin is 0% (0 μl) and the Vwmax is 65% (6.5 μl) (see column 6) and in the other solutions (see columns 2-5 of line B) the Vw is varied between Vwmin and Vwmax and, inversely, Vcrs is varied between Vcrsmin and Vcrsmax. In this example, Vcrs is incrementally varied between Vcrsmin and Vcrsmax and, inversely, Vw is incrementally varied between Vwmin and Vwmax (see column 2-5 of line B), the increment of Vw between Vwmin and Vwmax and of Vcrs between Vcrsmin and Vcrsmax remains the same, 13% (1.3 μl) between each solution. In this example, the number of individual soaking solutions i.e. 6 in the MPD series (line B) is the same as the number of individual soaking solutions x of the first series (line A); and the increment of Vcrs between Vcrsmin and Vcrsmax and of Vw between Vwmin and Vwmax is the same in the first (PEG400) series and in the MPD series.

The solutions were distributed in the wells of additional line (line B on FIG. 3) of an array thus constituting a second dimension. The solutions were preferably distributed following an order from the lowest Vw to the highest Vw but the order of the distribution in the array can be random.

On the same array of FIG. 3 (but another array could be used), another “first” series (line C with EG, C series) and its additional series (line D with Gly, D series) were prepared and distributed. The series of line C which was prepared with EG as cs can be designated as the first series according to the invention. Each soaking solutions of the C series comprises the same organic solvent (os) (DMSO) and the same compatible solute (EG) and the Vos 10% (10 I) and Vcs 10% (10 μl) remains the same in all solutions of the C series. However, the volume/proportion of Vcrs (CB) and Vw are changed in each solution of the first series, and one of the solution is composed of Vwmin is 0% (0 μl) and Vcrsmax is 80% (8 μl) (see column 1), and another solution is composed of Vcrsmin is 0% (0 μl) and the Vwmax is 80% (8 μl) (see column 6) and in the other solutions (see columns 2-5) the Vw is varied between Vwmin and Vwmax and, inversely, Vcrs is varied between Vcrsmin and Vcrsmax. In this example, Vcrs is incrementally varied between Vcrsmin and Vcrsmax and, inversely, Vw is incrementally varied between Vwmin and Vwmax in any additional solutions (see column 2-5 of line C) and in any additional solutions, the increment of Vw between Vwmin and Vwmax and of Vcrs between Vcrsmin and Vcrsmax remains the same, 16% (1.6 μl) between each solution of the first series.

The solutions were distributed in the wells of a line (line C on FIG. 3) of an array thus constituting a first dimension. The solutions were preferably distributed following an order from the lowest Vw to the highest Vw but the order of the distribution in the array can be random.

Simultaneously or not, the following step were performed as follows

Preparation of an additional series of 6 individual soaking solutions by mixing os, w, crs (CB: crystallization buffer) and cs. In this example of FIG. 3, the number of individual soaking solutions 6 is conserved and is the same as the number of individual soaking solutions of the first series. In the additional series (line D, series D), os is the same as in the first series (DMSO), but the cs is changed (Gly). Within the D series, each soaking solutions of the first series comprises the same organic solvent (os) (DMSO) and the same compatible solute (cs) (Gly) and the Vos and Vcs remains the same in all solutions of the D series. However, the volume/proportion of Vcrs (CB) and Vw are changed in each solution of the D series, and one of the solution is composed of Vwmin is 0% (0 μl) and Vcrsmax is 80% (8 μl) (see column 1), and another solution is composed of Vcrsmin is 0% (0 μl) and the Vwmax is 80% (8 μl) (see column 6) and in the other solutions (see columns 2-5 of line D) the Vw is varied between Vwmin and Vwmax and, inversely, Vcrs is varied between Vcrsmin and Vcrsmax. In this example, Vcrs is incrementally varied between Vcrsmin and Vcrsmax and, inversely, Vw is incrementally varied between Vwmin and Vwmax (see column 2-5 of line D), the increment of Vw between Vwmin and Vwmax and of Vcrs between Vcrsmin and Vcrsmax remains the same, 16% (1.6 μl) between each solution. In this example, the number of individual soaking solutions i.e. 6 in the MPD series (line D) is the same as the number of individual soaking solutions x of the first series (line C); and the increment of Vcrs between Vcrsmin and Vcrsmax and of Vw between Vwmin and Vwmax is the same in the first (EG) series and in the Gly series.

Then at least one PKA crystal prepared in a) above was placed in each compartments of the array of FIG. 3. The crystal was placed under microscope for control by visual analysis.

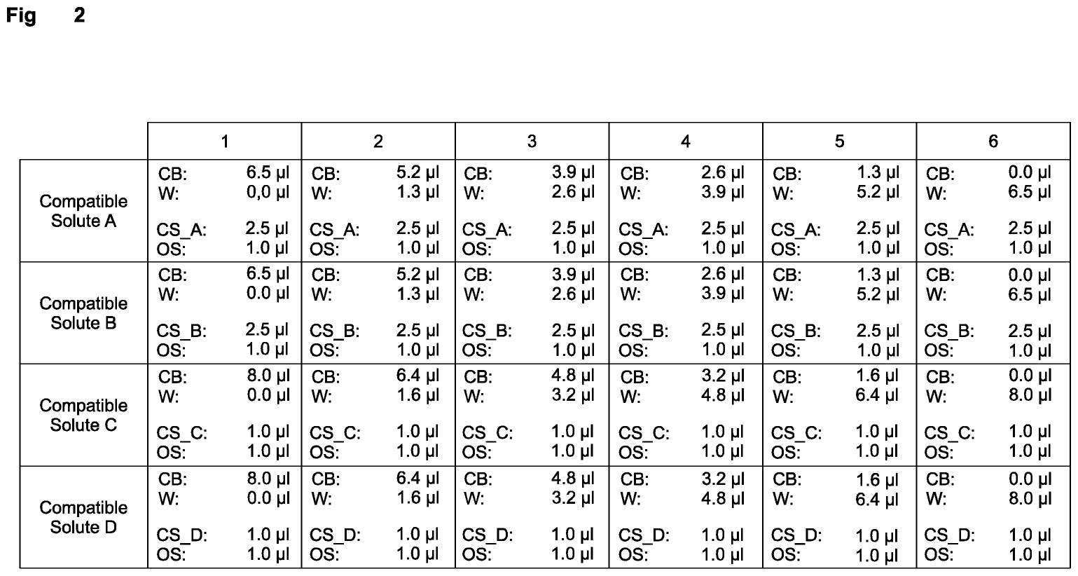

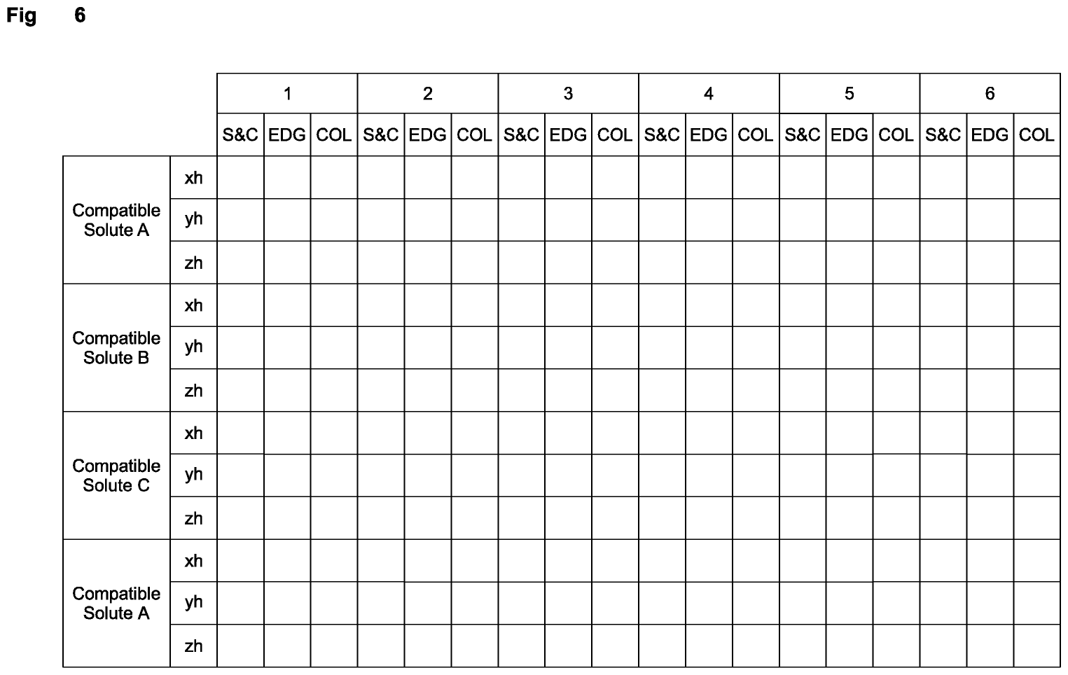

After the transfer of the crystal(s) in each well every well was photographed for visual control and present crystals characterized immediately after the transfer (0 h), after 1 h and 24 h. Pictures of crystals after 24 h are shown in FIG. 4 and the results of the visual inspection of the surfaces, edges and colours in FIG. 8 (data at 0 h and after 1 h are not shown). FIG. 4 shows clearly that after 24 h crystals are still present in row A in wells 1-6, in row B in wells 1-4 and in row C in well 3. Some remnants of crystals are visible in row B in wells 5 and 6 and in row C in wells 2 and 4 where these crystal remnants exhibit signs of advanced dissolution. In row C in wells 1, 5 and 6 as well as in row D no crystals are left.

From visual inspection crystals, the solutions suitable for soaking of the crystal were selected. Crystals in wells 1 to 4 in the first row A (PEG400) appeared suitable for diffraction tests as well as crystals in wells 1 and 2 in row B (MPD)(FIGS. 4 and 8). From visual inspection it appeared not expectable that crystals in well 5 and 6 in row A, in wells 3 and 4 in row B and crystals from the C row will diffract and therefore these solutions were not selected.

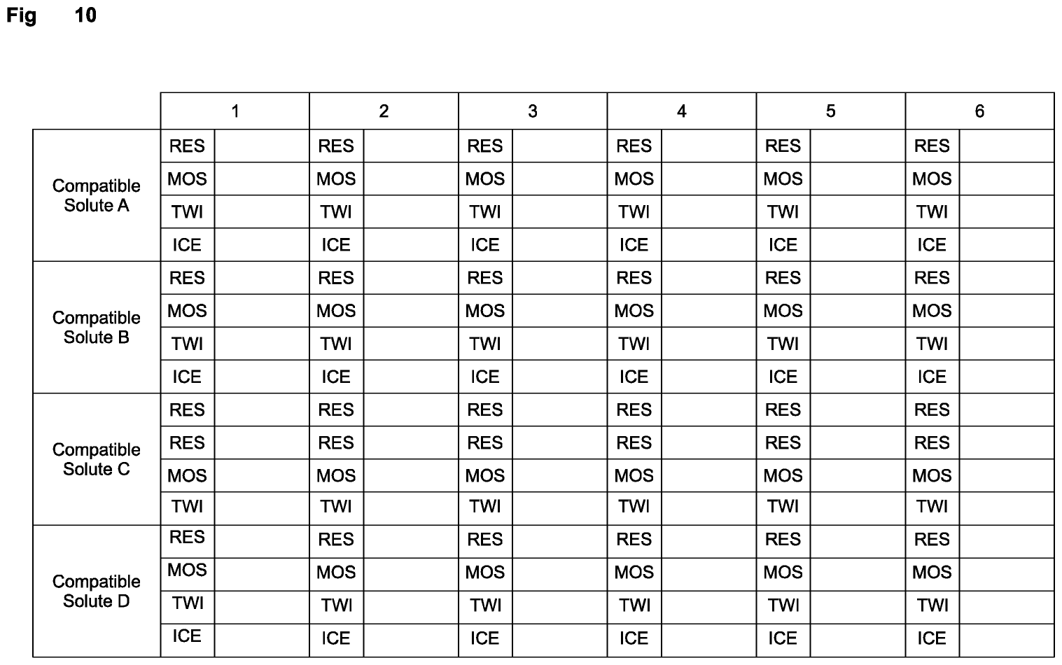

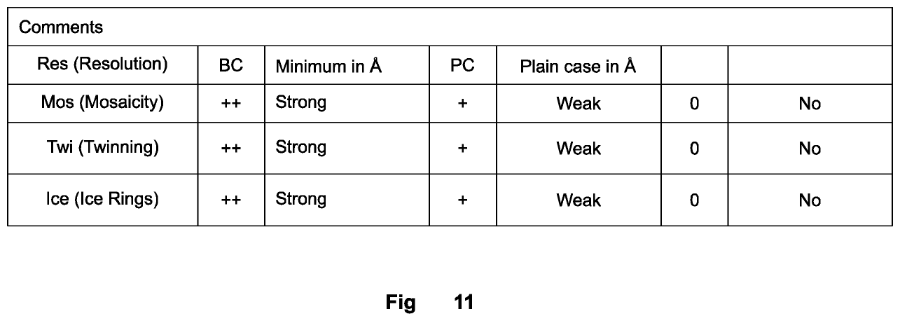

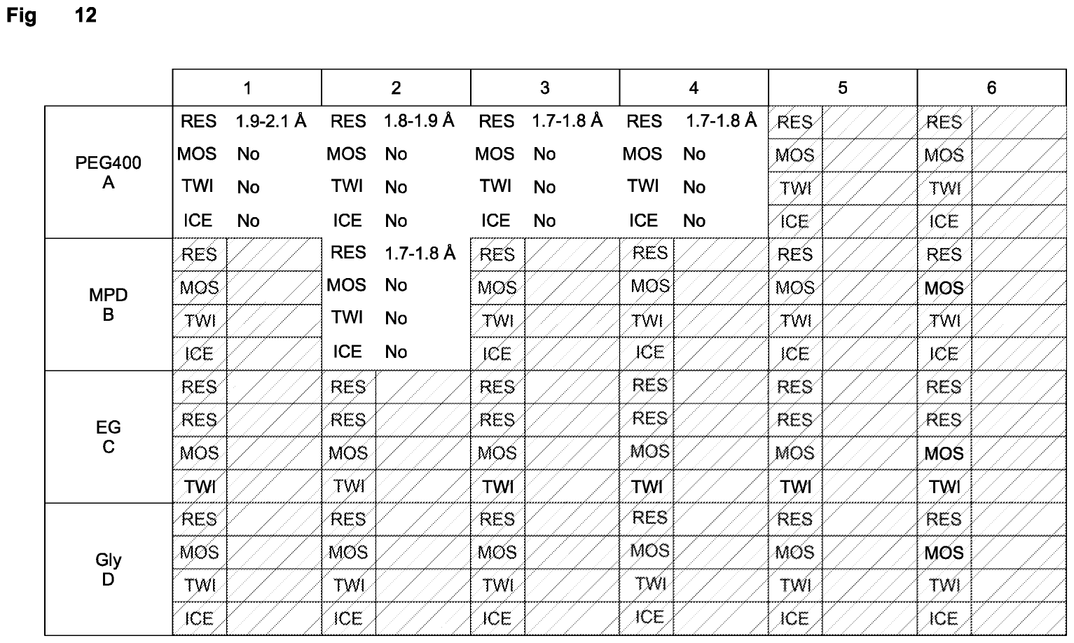

For further controls and selections, the crystals were harvested using cryo-loops, vitrified in liquid nitrogen and subjected to an inhouse x-ray measurement. For each crystal two perpendicular diffraction pattern were measured (position 0° and 90°). The results as exhibited in FIG. 12 show good diffraction quality for the crystals obtained from wells 1 to 4 in row A and wells 2 in row B. As expected, crystals from well 5 and 6 in row A, from wells 3 and 4 in row B and crystals from row C did not show diffraction. Against expectations, crystals from well 1 in row B did not show diffraction, too. The choice of the compatible solutes, organic solvent and the range of concentrations proved suitable to determine the scope of parameters affecting the protein's crystal integrity, stability and quality, respectively. In FIG. 13, the resulting suitable conditions are mapped on the scheme and have been highlighted. The mapping reveals a broad spectrum (4 solutions) of suitable conditions when using PEG400 and one single condition when using MPD.

Fragment Screening on PKA.

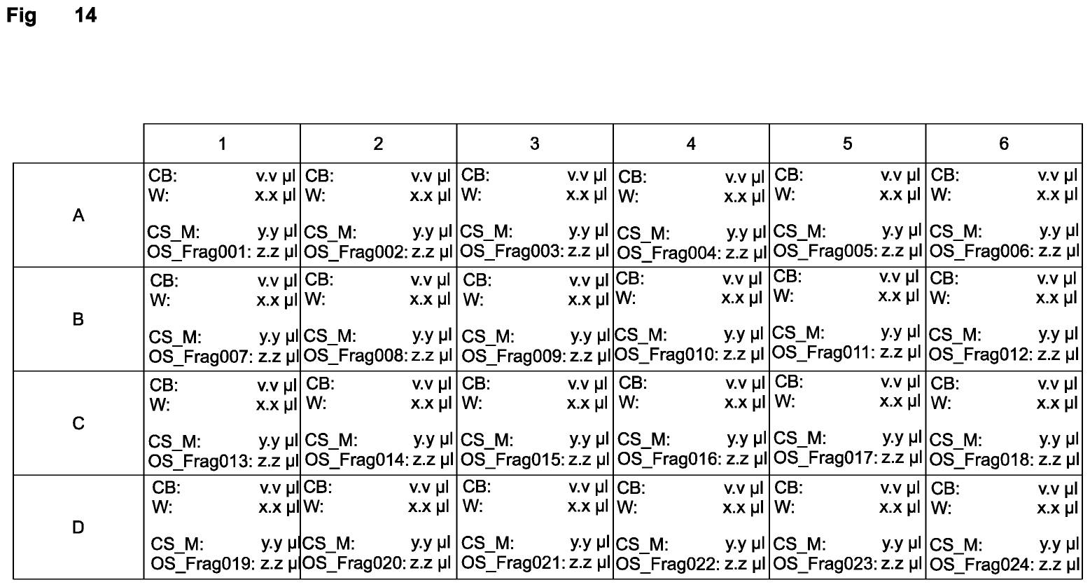

For the fragment screen on PKA the composition of the soaking solution of the well 1 in row A was selected (indicated with a star in FIG. 13). The soaking solution with the same proportion of cs (25% of PEG400), crs (65% of CB) and os (10% of DMSO) was prepared. Several 24-well sitting drop experimental plates were filled with the solution (FIG. 16). Instead of DMSO alone, 1 M fragment in DMSO was used (end concentration in each well 100 mM). Afterwards, freshly prepared crystals were transferred to the wells using cryo-loops. Overall, the experiment subjected PKA crystals to 87 different fragments.

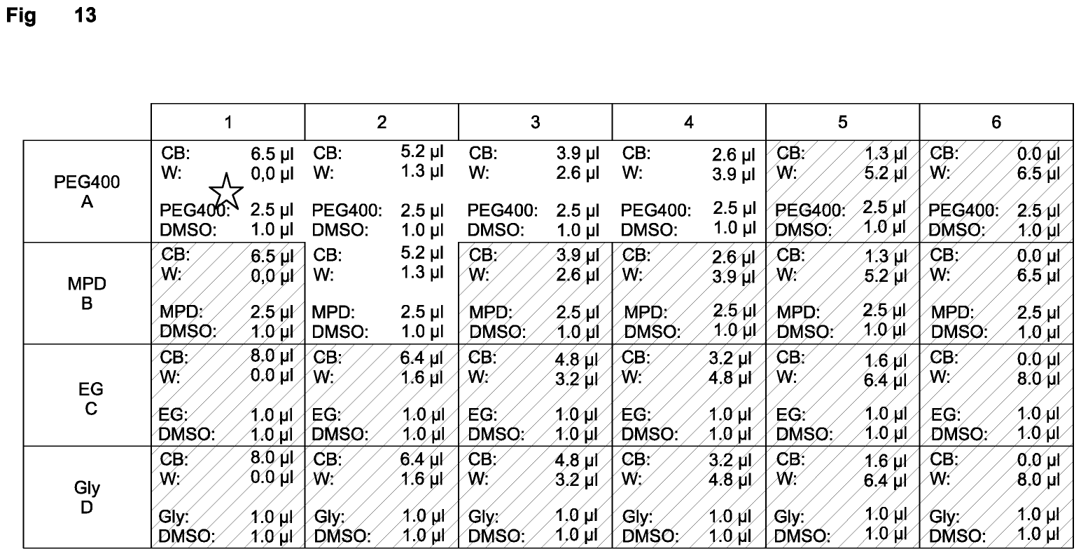

On FIG. 15, another example of plate for screening is shown using the selected composition of an alternative suitable soaking solution made of 25% cs (PEG400), 39% crs (CB), 26% water and 10% os (DMSO) selected according to the method of the invention—see line A column 3 of FIG. 13.

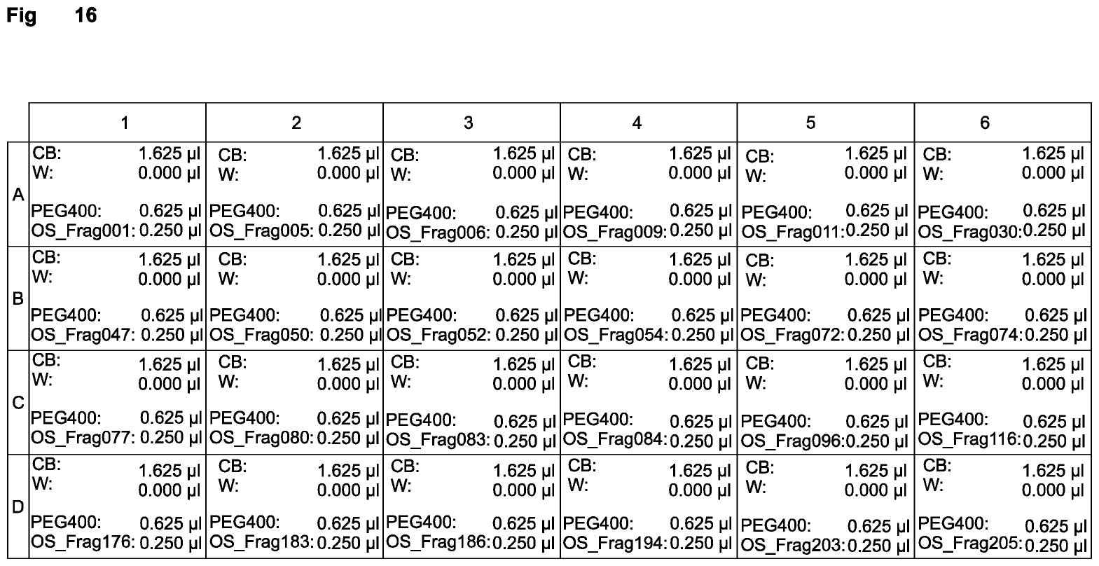

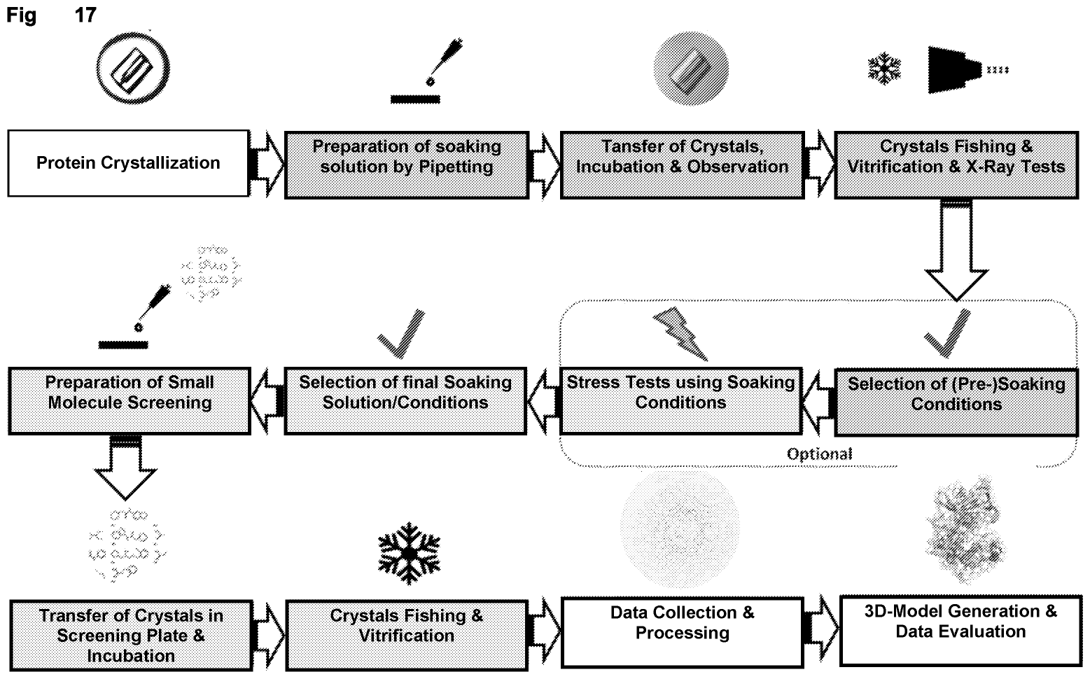

After 24 h of soaking time the soaked crystals were harvested using cryo-loops and vitrified in liquid nitrogen. Data collection took place at a synchrotron. Data evaluation yielded 55 binders at resolutions of 1.3-1.8 Å. FIG. 18 shows the distribution of hits over the example plate of FIG. 16. The refinement of according data yielded 3D-models of the interaction of 55 fragments with PKA at different interaction sites from which 27 fragments bind to the ATP-binding pocket. FIG. 19 shows an overlay of the protein with respective binders. The study is just one example of a very successful application of the method of the invention to determine soaking parameters for a small molecule screening (in this case with fragments). Among the hits were several unexpected chemotypes for kinase binders. That proves that the method of the invention technology is a very successful means to perform rapid and reliable small molecule-screenings and delivers high-quality data.

### Example 2—A Rule-Based Method of Selection of the Suitable Soaking Solution and Subsequent Small Molecule Screening

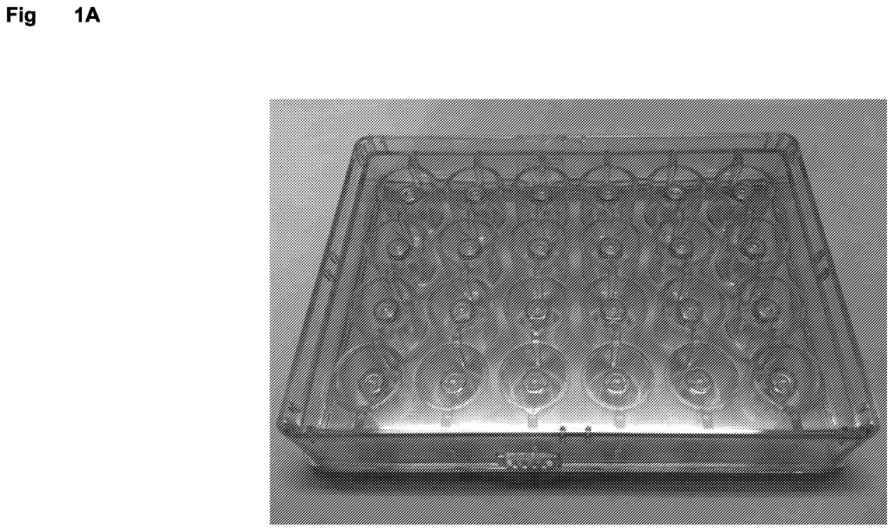

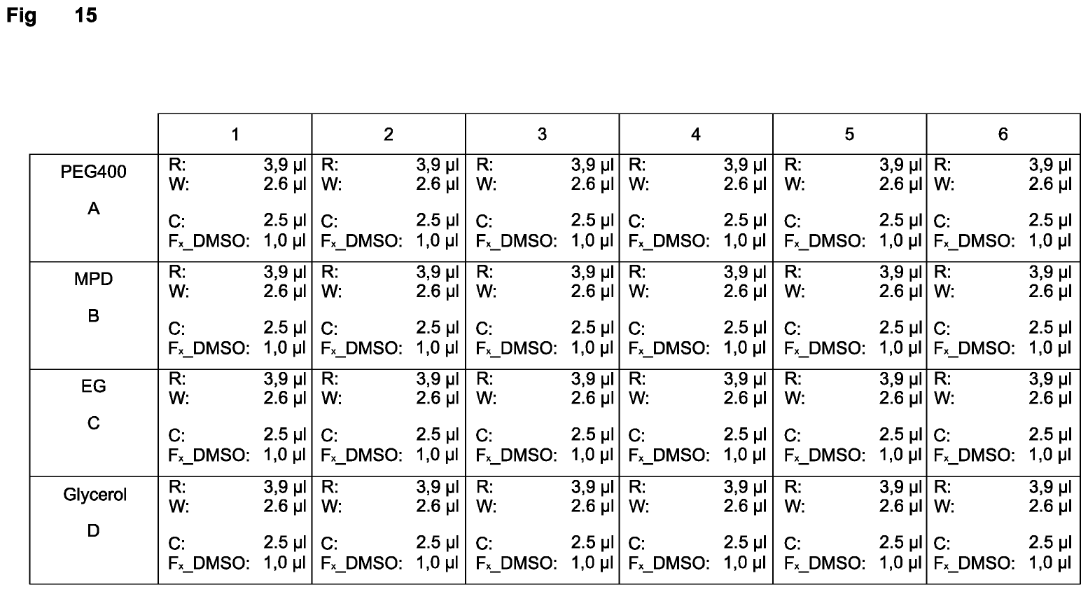

In order to perform a small molecule fragment screening, crystals of the protein of interest (POI) must be obtained (step a of workflow in FIG. 17). The crystallization is conducted according to the vapor diffusion approach in commercially available 24-well sitting drop crystallization plates (or any other plate format)(FIG. 1). For this purpose, the reservoirs of each 24-well plate used are filled with a buffer according to a beforehand established crystallization protocol. Furthermore, a crystallization drop is prepared in the indentation on top of the column of each of the wells by adding some volume of a protein solution to some volume of the buffer which is also used in the reservoir. Usually, but not always, the volumes are in a ratio of 1:1 (FIG. 1). The resulting protein concentration is usually, but not always, in the range of 2 to 30 mg/ml but can also reach concentrations of 250 mg/ml or more. Afterwards the wells are sealed using an adhesive film, so called sealing tape. All these steps are conducted in an according to the crystallization protocol correctly tempered laboratory, usually at 4° C., 10° C., 18° C., 20° C. The plates are stored on a shelf in this laboratory for the time needed for crystallization, usually a few days to a few weeks.

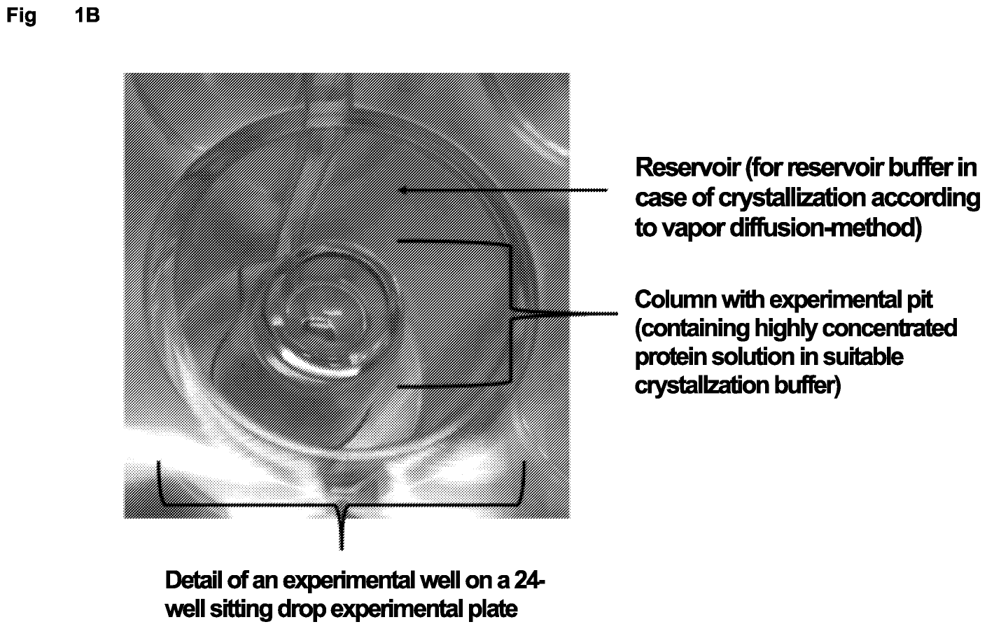

After the crystallization process is finished, a selection of the soaking solution according to the present invention is conducted in order to map conditions for a small molecule fragment screening (same temperature). For this purpose, a 24-well crystallization plate (or any other plate format) is utilized, and the indentation of each well are provided with possible soaking solutions prepared according to the present invention as is exemplified in FIG. 1-3 (step b of workflow in FIG. 17). Subsequently, the film used for sealing the 24-well plates is removed from the plates used for crystallization of the POI. The meanwhile grown crystals are removed from their growth solutions using a special laboratory tool usually equipped with a nylon loop for crystal picking or any other appropriate tool for the so-called “crystal fishing” and transferred to the possible soaking solutions in the indentations of the new 24-well plate that was prepared according to an instantiation of the protocol (step c of workflow in FIG. 17). These plates are also sealed using sealing tape.

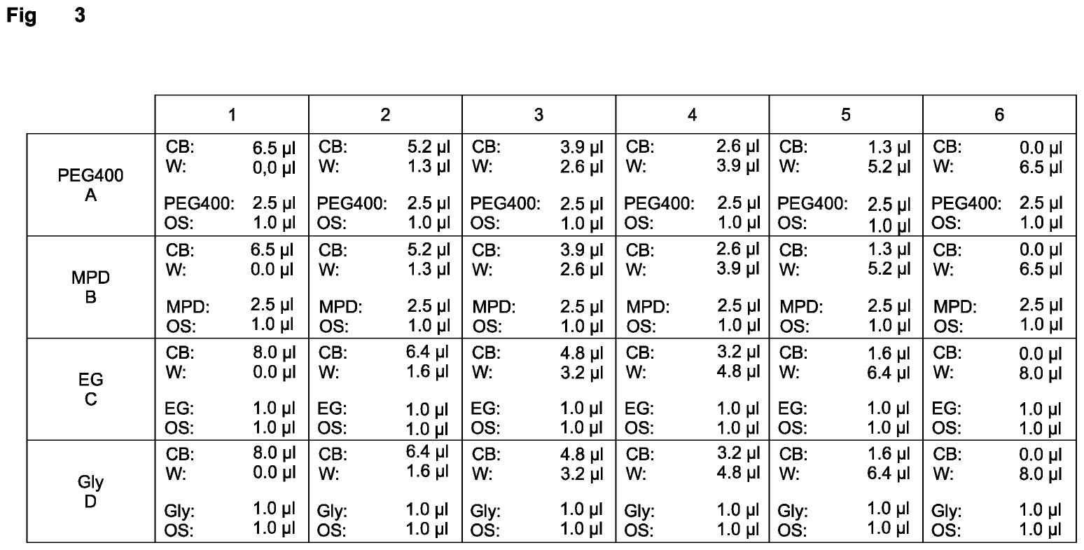

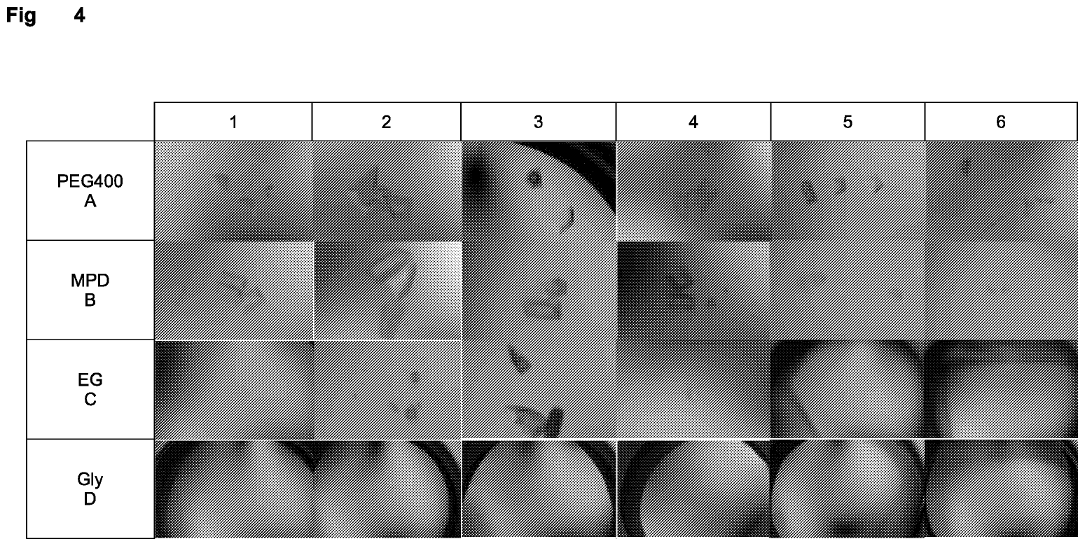

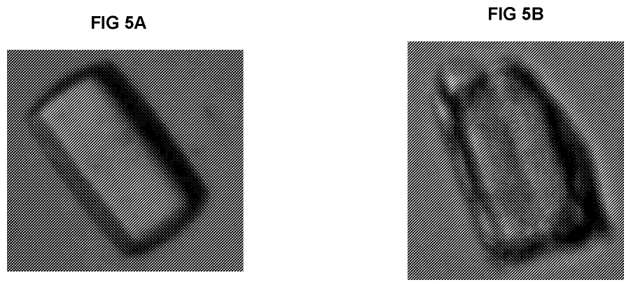

The purpose of the next steps is to control the crystals quality, the maximal soaking time and a proper relation between soaking time and crystal quality for small molecule fragment screenings. Within a certain time, usually the next 24 hours, pictures of each indentation are taken under a microscope at least 1 time, usually three times, sometimes more, under a microscope in order to document the crystals conditions for example immediately after the transfer, after 1 hour and after 24 hours (FIG. 4-5). In parallel, usually notes are taken in order to supplement the information in the pictures. (A template for such notes is exemplified in FIG. 6). Notes refer to different types of cracks crystals can show (longitudinal, lateral, crisscross), to the appearance of the crystals surface (e.g. smooth, rough) or edges (e.g. sharp, roundish) and their behavior in polarized light (strong color, some color, no color) (FIG. 8, exemplified for 24 h). In the most cases not all of the crystals survive in all the conditions. Some crystals dissolve over time or are damaged while other crystals are in good condition. Therefore, an X-Ray evaluation of the crystal's quality is recommended (step d of workflow in FIG. 17). For this purpose, the sealing tape is removed from the plate in order to transfer the remaining crystals from the plate into liquid nitrogen or another vitrification medium. In case that the X-Ray study is conducted at room temperature, previous vitrification is not required. The crystals' vitrification takes place in the nylon loops mentioned before or any other equivalent transfer-equipment like litho-loops or capillaries. The vitrified crystals in the nylon loops or other transfer-equipment are stored in so called “cryo-vials” under liquid nitrogen or another vitrification medium until they are subjected to an X-Ray examination. Crystals can also be stored in any other appropriate storage container.

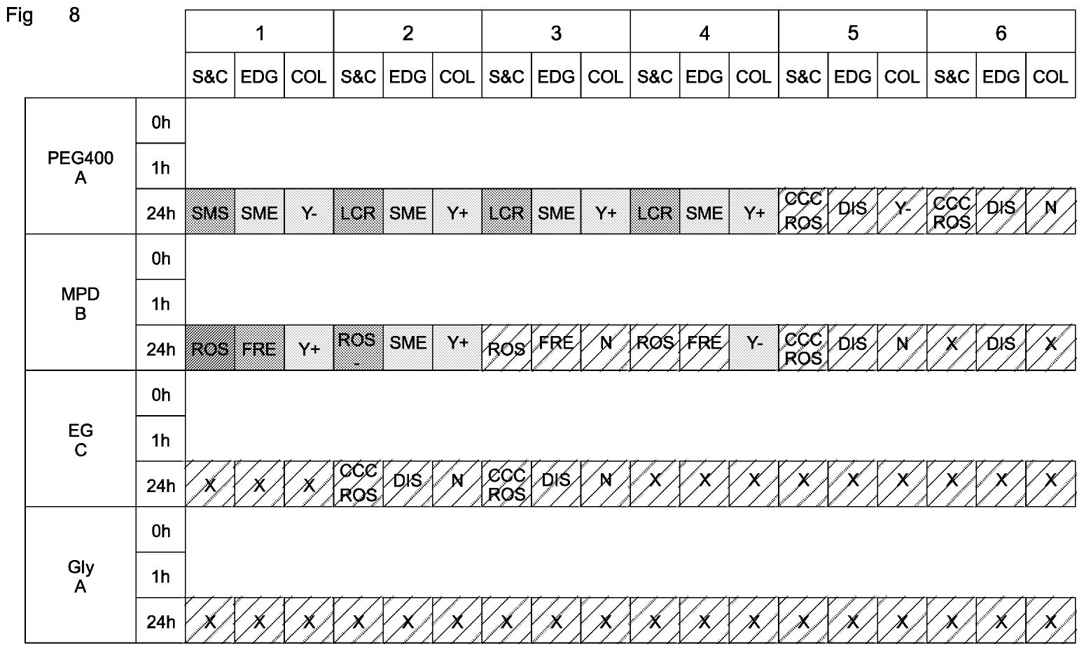

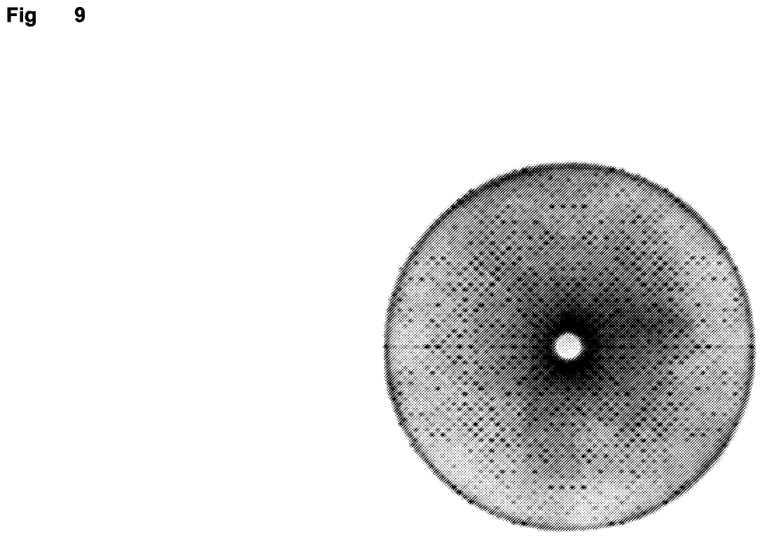

For the X-Ray examination the crystals in the nylon loops or other transfer-equipment are removed from the cryo-vials or other storage containers and mounted on an X-Ray machine in a so-called cryo-stream that maintains the cryogenic temperature around the protein crystal. In case of X-Ray studies at room temperature, no cryo-stream is required. Afterwards, X-Ray data is collected. Using an inhouse X-Ray source two to several pictures are collected. At a synchrotron source a whole data set is collected (preferably on a template of FIG. 10-11), due to its rapid data collection. The raw data (FIG. 12) is evaluated regarding resolution, and mosaicity, the appearance of artifact like data originating from unwanted ice crystals in the protein crystal and sometimes a phenomenon called “twinning” (FIG. 5B). In case of data collected at a synchrotron, the appearance of the so called “electron density” is judged by an experienced crystallographer as suitable or not suitable. Based on this data a decision is made on which soaking condition or conditions is/are to be chosen for a small molecule fragment screening (FIG. 13, marked with a star; step e/g of workflow in FIG. 17). In contrast to traditional optimizations of soaking solutions which result in one single soaking condition, the rule-based method of the invention usually results in a spectrum of soaking conditions. From this spectrum the experimenter can choose alternative soaking solutions to face detrimental effects on the crystal that may be caused by for example special properties of small molecule fragments or pH changes etc.

Optionally, at this stage of the workflow stress tests can be conducted (step f of workflow in FIG. 17). For this purpose, one or more of the previously obtained soaking solutions is/are selected and prepared in a new 24-well plate. Afterwards, crystals are transferred to these solutions. Instead of pure DMSO, small molecules, for example molecular probes or small molecule fragments, for stress tests dissolved in DMSO are used. They exert different kinds of stress such as pH-stress or osmotic stress on the crystals. Based on the subsequently performed data evaluation (same as in step d of workflow in FIG. 6) one or more final soaking solutions can be chosen.

Usually, a decision is made for one soaking condition which is subsequently used for the small molecule fragment screening. For a small molecule fragment screening comprising 300 small molecule fragments 13 of the 24-well plates are prepared by pipetting the respective soaking solution composition of the chosen soaking solution without DMSO. Instead of pure DMSO, 300 small molecule species at concentrations of usually 1M dissolved in DMSO are used and added to the prepared 300 wells. This results in final concentrations of the small molecule fragments in each soaking solution of usually 100 mM but may be higher or lower (FIG. 15-16; step h of workflow in FIG. 17). Following that, usually two newly prepared crystals (sometimes just one or more than two) are transferred to each soaking solution and soaked for usually 24 hours (step i of workflow in FIG. 17). Then, the crystals are fished, transferred to liquid nitrogen for vitrification (step j of workflow in FIG. 17), stored in cryo-vials in Uni-pucks or some other applicable storage device and sent to a synchrotron facility, where data collection takes place (step k of workflow in FIG. 17). In case of X-Ray studies at room temperature no vitrification is required.

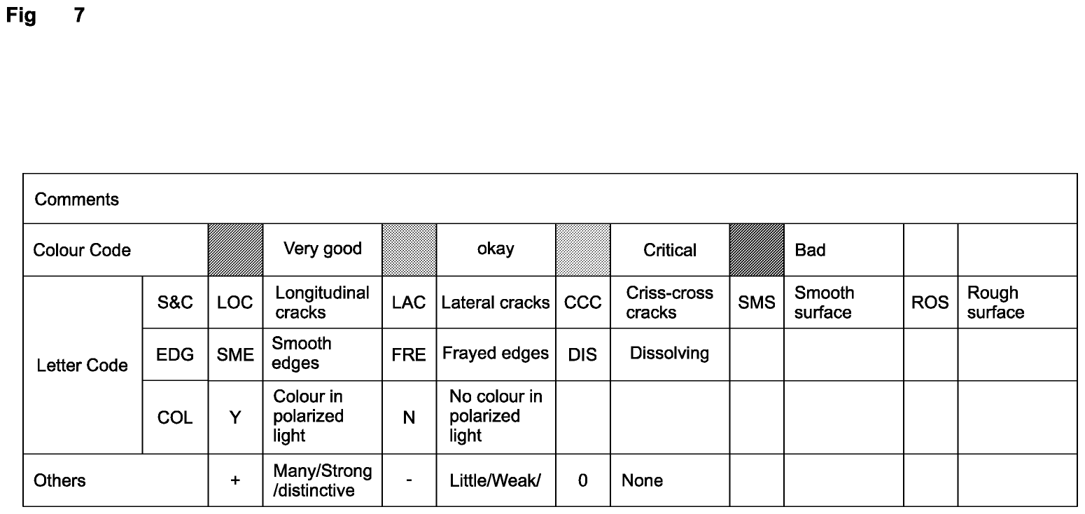

In data collection individual data sets for all crystals are obtained comprising of several hundreds to thousands of pictures showing reflections of the X-Ray beams (exemplified in FIG. 9). These reflections stem from interactions between X-Ray and electrons and, therefore, can be exploited to reconstruct the so-called electron densities. Such electron densities define the space in the crystals where electrons are located and are representative for the protein's geometry. Using computational methods important information are extracted from these data sets. To reconstruct the geometry further information is needed, the so-called phases, that are not extractable from this kind of experiment without heavy atom derivatization. This information can be derived in an approach called molecular replacement from previously obtained structural solutions of the same or very similar proteins. The result is a 3-dimensional model of the protein's geometry according to the positions of all the electrons in the protein (FIG. 19). This electron density is merged with the known sequence of amino acids of the respective protein and positions of structurally fixed water molecules are identified. This solution for the structure is a model that must be optimized in several structure refinement cycles in order to obtain a convincing fit between the measured data and the model. The result is an atomically resolved representation of the geometry of the target (FIG. 19; step I of workflow in FIG. 17).

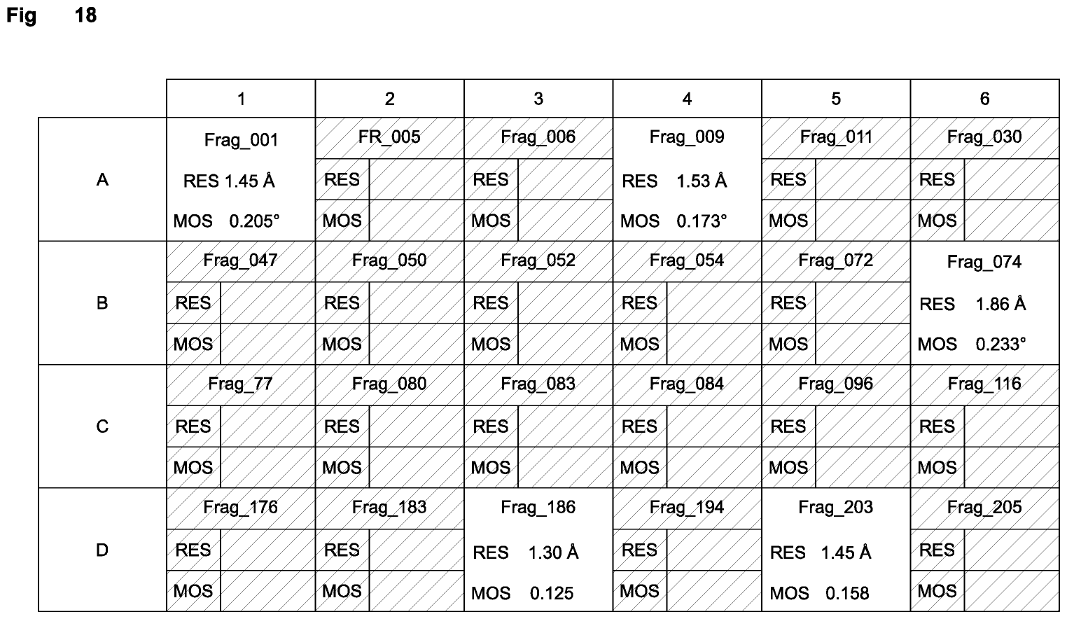

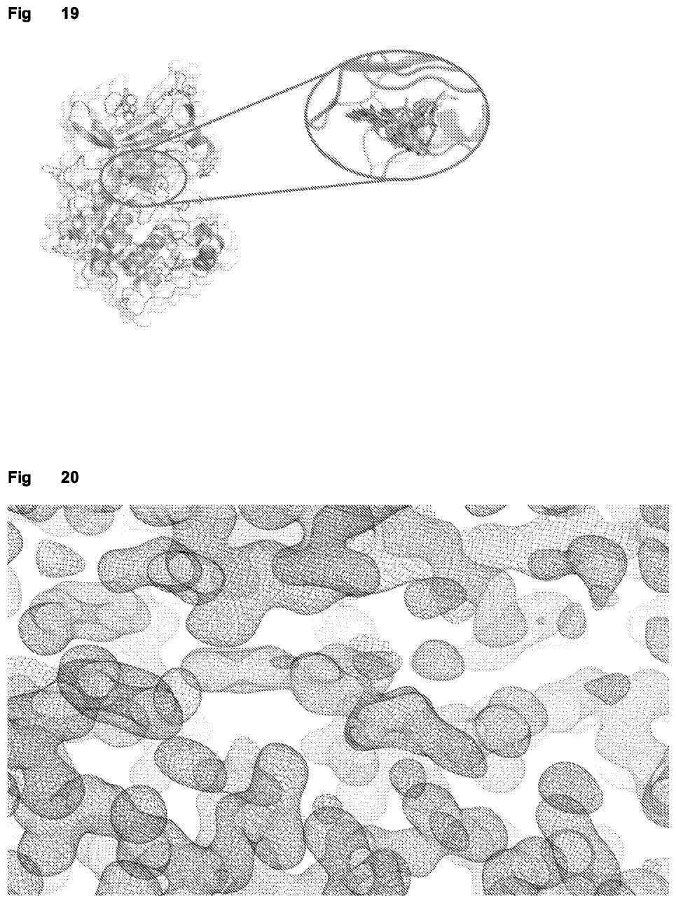

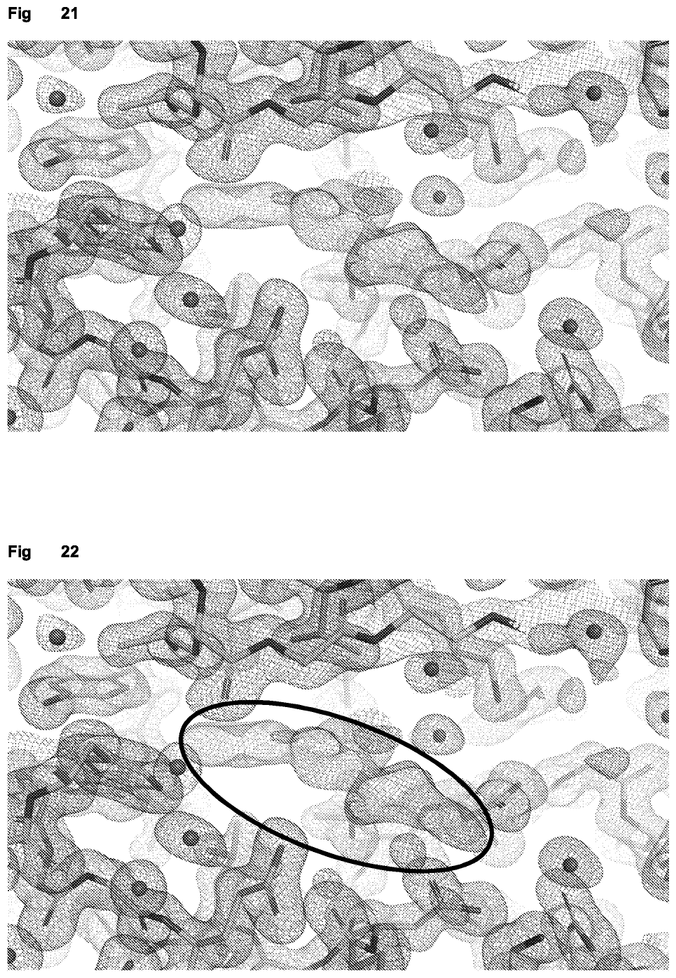

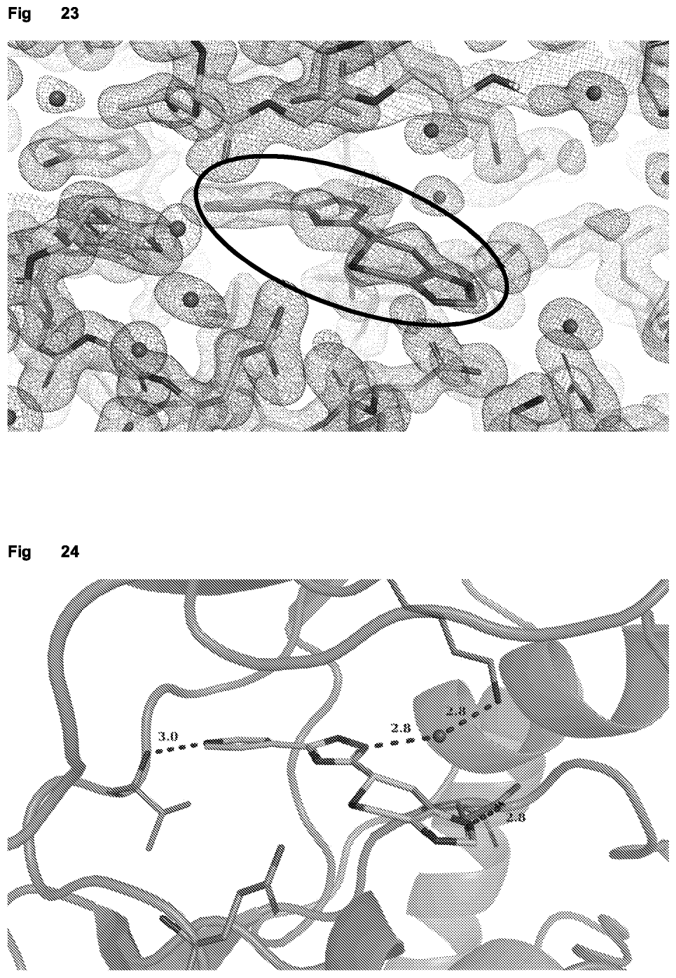

Each of these structures is examined in order to identify electron density (FIG. 20) that can be assigned to the special small molecule fragment that was used for soaking of this very crystal (FIG. 21-22). If this is the case, this small molecule fragment is judged as a so-called “hit”. Examination of all data sets reveals all small molecule fragment-binders (FIG. 23 maps the hits on the experimental layout on one plate of the screening encompassing five hits). Data sets without additional electron density that is assignable to respective small molecule fragments are discarded. Further refinement of each of these “hit”-data sets reveal the exact geometry of the interaction between the protein and the respective small molecule fragments (FIG. 24). Therefore, the described process provides validated knowledge about small molecule fragments that bind to the protein target and delivers structurally elucidated binding modes that can be used for medicinal-chemical optimization processes. Instead of small molecule fragments also more drug-size molecules can be used for example to validate follow-up compounds resulting from medicinal-chemical optimization. Furthermore, special molecular probes can be used to map properties of the binding sites or to obtain information about putative pharmacophore patterns etc.

1. Method of selecting a soaking solution for soaking the crystal form of a biological macromolecule wherein the soaking solution comprises an organic solvent (os), a compatible solute (cs), and a crystallization solution (crs) and/or water (w); comprising the steps of
1) preparing a first series of 1n to xn individual soaking solutions by mixing os, cs, crs and w wherein:
a) x is at least 2
b) each soaking solutions of the first series comprises the same organic solvent (os), the same compatible solute (cs), and the same crystallization solution (crs), and
c) Vos and Vcs are the same in each solution, or, alternatively, Mos/Mcs is the same in each solution, and
d) Vcrs and Vw are changed in each solution, and
in the solution in, V_{crs} is the minimum V_{crs} (V_{crs} min) and the V_{w} is the maximum V_{w} (V_{w} max), and
in the solution xn, V_{w} is the minimum V_{w} (V_{w} min) and the V_{crs} is the maximum V_{crs}(V_{crs} max), and
when x>2, in any additional solutions between in and xn, the V_{crs} is varied between V_{crs} min and V_{crs} max and, inversely, Vw is varied between V_{w} min and V_{w} max
2)transferring at least one crystal of the biological macromolecular crystal per compartment into x compartments, each compartment comprising one of the solutions prepared in step 1); or transferring each solution prepared in step 1) into x compartments, each compartment comprising at least one crystal of the biological macromolecular crystal from its crystallization solution.
3) controlling the crystal in each compartment
4) selecting the soaking solution(s) which is/are suitable for soaking the crystal;
wherein the compatible solute is selected from the group comprising polyols, amino acids, methylamines, and mixtures thereof, and
wherein the organic solvent is selected from the group comprising liquid carbohydrates, protic or aprotic, of low reactivity, that can serve to solve small molecules and, mixtures thereof, and wherein the crystallization solution crs is obtained from the crystallization process of said crystal or is equivalent thereto in terms of osmotic net pressure, permittivity, solubility of the according biological macromolecule, and effect on its structural conformation, comprising pH and buffer materials, additives, and precipitants.

2. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably, said compatible solute (cs) is a poly(oxyethylene) and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the poly(oxyethylene)s polyethylene glycol 400, polyethylene glycol 600 or a mixture thereof and said organic solvent (os) is selected from the group comprising dimethyl sulfoxide, 1,4-dioxane, dimethylformamide, methanol, ethanol, or a mixture thereof and
wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous solution V_{crs} to the combined volumes of organic solvent Vos and compatible solute Vcs is 1:1000000 to 1000000:1, preferably between 99:1 to 1:99 and most preferably between 85:15 to 1:1 (V_{crs}:(Vos; Vcs)).

3. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is a polyol or a mixture thereof, and said organic solvent is a (alkylated) sulfoxide, cyclic non-aromatic ether, straight or branched chain monohydric aliphatic alcohol, alkylated formamide, or a mixture thereof,
more preferably, said compatible solute (cs) is a polyhydric alcohol or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute (cs) is selected from the group comprising the polyhydric alcohols propane-1,2,3-triol (glycerol), ethane-1,2-diol (ethylene glycol), 2-Methylpentane-2,4-diol (2-Methylpentane-2,4-diol),(3R,4S,5S,6R)-2-(2,3-dihydroxypropoxy)-6-(hydroxymethyl)oxane-3,4,5-triol (1-Glucosylglycerol) or a mixture thereof, and said organic solvent (os) is selected from the group comprising dimethyl sulfoxide, 1,4-dioxane, dimethylformamide, methanol, ethanol, or a mixture thereof and
wherein the ratio of volume compatible solute Vcs to volume organic solvent Vos is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous solution V_{crs} to the combined volumes of organic solvent Vos and compatible solute Vcs is 1:1000000 to 1000000:1, preferably between 99:1 to 1:99 and most preferably between 85:15 to 1:1 (V_{crs}:(Vos; Vcs)).

4. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is a polyol a or a mixture thereof, and said organic solvent (os) is a liquid carbohydrate protic or aprotic, of low reactivity, or a mixture thereof, that can serve to solve small molecules,
more preferably said compatible solute (cs) is a mono-, di-, tri, oligo- or polysaccharide or a mixture thereof and said organic solvent (os) is selected from the group comprising a (alkylated) sulfoxide, cyclic non-aromatic ether, straight or branched chain monohydric aliphatic alcohol, alkylated formamide or a mixture thereof,
most preferably, the compatible solute cs is selected from the group comprising the saccharides (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (trehalose) or β-D-Fructofuranosyl α-D-glucopyranoside (sucrose) or a mixtures thereof, and said organic solvent (os) is selected from the group comprising dimethyl sulfoxide, 1,4-dioxane, dimethylformamide, methanol, ethanol, or a mixture thereof and
wherein the molar ratio of compatible solute to organic solvent is the same and the molar ratio is 1:10000 to 10000:1, preferably between 100:1 to 1:100 and more preferably between 10:1 to 1:10,
and wherein the ratio of volume crystallization solution or equivalent aqueous solution V_{crs} to the combined volumes of organic solvent Vos and compatible solute Vcs is 1:1000000 to 1000000:1, preferably between 100:1 to 1:100 and most preferably between 85:15 to 1:1 (V_{crs}: (Vos; Vcs)).

5. Method of selecting the composition of a soaking solution according to claim 1, wherein said soaking solution comprises a crystallization solution (crs) or an equivalent solution thereto and an organic solvent (os) and a compatible solute (cs),
wherein said compatible solute (cs) is a methylamine or a mixture thereof and said organic solvent (os) is liquid carbohydrate protic or aprotic, of low reactivity, or a mixture thereof that can serve to solve small molecules,
more preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is a (alkylated) sulfoxide, cyclic non-aromatic ether, straight or branched chain monohydric aliphatic alcohol, alkylated formamide, or a mixture thereof,
most preferably said compatible solute (cs) is selected from the group comprising N,N-dimethylmethanamine oxide (Trimethylamine N-oxide or TMAO), 2-trimethylammonioacetate (trimethylglycine or betaine), 2-(Methylamino)acetic acid (N-methylglycine or sarcosine), or a mixture thereof and said organic solvent (os) is selected from the group comprising dimethyl sulfoxide, 1,4-dioxane, dimethylformamide, methanol, ethanol, or a mixture thereof and
wherein the molar ratio of organic solvent (os) to compatible solute (cs) Mos/cs is the same and the molar ratio Mos/cs is 1:10000 to 10000:1, preferably between 100:1 to 1:100 and more preferably between 10:1 to 1:10,
and wherein the ratio of volume crystallization solution or equivalent aqueous solution V_{crs} to the combined volumes of organic solvent Vos and compatible solute Vcs is 1:1000000 to 1000000:1, preferably between 100:1 to 1:100 and most preferably between 85:15 to 1:1 (V_{crs}:(Vos;Vcs)).

6. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is polyethylene glycol 400 and said organic solvent (os) is dimethyl sulfoxide,
wherein the ratio of volume Vcs polyethylene glycol 400 to volume Vos dimethyl sulfoxide is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous salt solution V_{crs} to the combined volumes of the organic solvent (os) dimethyl sulfoxide and the compatible solute (cs) polyethylene glycol 400 is 1:1000000 to 1000000:1, preferably between 99:1 to 1:99 and most preferably between 85:15 to 1:1 (V_{crs}:(Vos:Vcs)).

7. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is ethane-1,2-diol (ethylene glycol) and said organic solvent (os) is dimethyl sulfoxide,
wherein the ratio of volume Vcs ethane-1,2-diol (ethylene glycol) to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous salt solution V_{crs} to the combined volumes of the organic solvent (os) dimethyl sulfoxide and the compatible solute (cs) ethane-1,2-diol (ethylene glycol) is 1:1000000 to 1000000:1, preferably between 99:1 to 1:99 and most preferably between 85:15 to 1:1 (V_{crs}:(Vos:Vcs)).

8. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is propane-1,2,3-triol (glycerol) and said organic solvent (os) is dimethyl sulfoxide,
wherein the ratio of volume Vcs propane-1,2,3-triol (glycerol) to volume Vos dimethyl sulfoxide is the same and the volume ratio is 1:1000000 to 1000000:1, preferably between 8:1 to 1:8 and more preferably between 3:1 to 1:1 (Vcs:Vos);
and wherein the ratio of volume crystallization solution or equivalent aqueous salt solution V_{crs} to the combined volumes of the organic solvent (os) dimethyl sulfoxide and the compatible solute (cs) propane-1,2,3-triol (glycerol) is 1:1000000 to 1000000:1, preferably between 99:1 to 1:99 and most preferably between 85:15 to 1:1 (V_{crs}:(Vos:Vcs)).

9. Method of selecting the composition of a soaking solution according to claim 1, wherein said soaking solution comprises a crystallization solution (crs) or an equivalent aqueous salt solution thereto and an organic solvent (os) and a compatible solute (cs),
wherein said compatible solute (cs) is 2-methylpentane-2,4-diol (MPD) and said organic solvent (os) is dimethyl sulfoxide,
wherein the ratio of volume Vcs 2-methylpentane-2,4-diol (MPD) to volume Vos dimethyl sulfoxide is the same and the volume ratio is 1:1000000 to 1000000:1, preferably between 8:1 to 1:8 and more preferably between 3:1 to 1:1 (Vcs: Vos);
and wherein the ratio of volume crystallization solution or equivalent aqueous salt solution V_{crs} to the combined volumes of the organic solvent (os) dimethyl sulfoxide and the compatible solute (cs) 2-methylpentane-2,4-diol (MPD) is 1:1000000 to 1000000:1, preferably between 99:1 to 1:99 and most preferably between 85:15 to 1:1 (V_{crs}:(Vos:Vcs)).

10. Method of selecting the composition of a soaking solution of claim 1 further comprising the step of
1) preparing m series of 1n to yn individual soaking solutions by mixing os, cs, crs and w wherein m is at least 1 and wherein in each of the m series
a) y is at least 2
b) os and crs are the same as in the first series, and
c) Vos and Vcs are the same as in the first series, or, alternatively, Mos/Mcs is the same in the first series,
d) the cs is changed and is different from the cs of the first series, and
wherein in each individual solutions of each of the m series
e) Vos and Vcs are conserved, or, alternatively, Mos/Mcs is conserved, and
f) V_{crs} and Vw are changed in each solution, and
in the solution 1n, V_{crs} is the minimum V_{crs} (V_{crs} min) and the Vw is the maximum V_{w} (V_{w} max), and
in the solution yn, V_{w} is the minimum V_{w} (V_{w} min) and the V_{crs} is the maximum V_{crs}(V_{crs} max), and
when y>2, in any additional solutions between in and yn, the V_{crs} is varied between V_{crs} min and V_{crs} max and, inversely, V_{w} is varied between V_{w} min and V_{w} max
2) transferring at least one crystal of the biological macromolecular crystal per compartment into y compartments, each compartment comprising one of the solutions prepared in step 1); or transferring each solution prepared in step 1) into y compartments, each compartment comprising at least one crystal of the biological macromolecular crystal from its crystallization solution.

11. Method of selecting the composition of a soaking solution of claim 1 wherein an equivalent aqueous salt solution different from the crystallization solution is used, and wherein the salt selected from the group comprising disodium; 2,3-dihydroxybutanedioate (sodium tartrate), (2R,3R)-2,3-dihydroxybutanedioate (dipotassium tartrate), azanium; phosphates (ammonium phosphates), diazanium; sulfate (ammonium sulfate), triazanium; 2-hydroxypropane-1,2,3-tricarboxylate (ammonium citrate), sodium; acetate (sodium ethanoate), azanium; acetate (ammonium acetate), dilithium; sulfate, lithium; chloride, lithium; acetate, lithium; formate, lithium; nitrate, sodium; nitrate, sodium; chloride, potassium; chloride, sodium; formate, monophosphate from sodium and potassium, di- and polyphosphates from sodium and potassium, sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate)), magnesium; diformate, magnesium; dichloride, magnesium; sulfate, calcium; dichloride; dihydrate, disodium; butanedioate (sodium succinate), cadmium(II) sulfate (cadmium sulfate), disodium; propanedioate (sodium malonate), magnesium; diacetate (magnesium acetate), zinc; diacetate (zinc acetate), calcium; diacetate (calcium acetate) and wherein the salt for the equivalent solution is preferably selected from the group comprising lithium; acetate, lithium; chloride, lithium; formate, lithium; nitrate, dilithium; sulfate, sodium; chloride, potassium; chloride, magnesium; dichloride, diazanium; sulfate (ammonium sulfate), sodium; acetate (sodium ethanoate), sodium citrates (sodium citrates as sodium dihydrogen 2-hydroxypropane-1,2,3-tricarboxylate (monosodium citrate), disodium hydrogen 2-hydroxypropane-1,2,3-tricarboxylate (disodium citrate), trisodium 2-hydroxypropane-1,2,3-tricarboxylate (trisodium citrate), magnesium; diformate, sodium; nitrate.

12. Method of selecting the composition of a soaking solution of claim 1 wherein the os contains small molecules including small fragments and small probes to be analyzed, which diffuse into the biological macromolecular crystal.

13. Method of small molecule screening for a biological macromolecular crystal wherein the screening is performed in a soaking solution selected according to the method of claim 12.

14. Soaking solution obtainable by the method of selecting the composition of a soaking solution as claimed in claim 1.

15. (canceled)

16. Array arranged to perform the method as claimed in claim 1, wherein said array comprises a first dimension of at least two individual soaking solutions (1n to xn) and a second dimension of at least two individual soaking solutions (1m to ym) wherein each of said soaking solutions is located in a separated compartment of said array, and wherein each of said soaking solution comprises an organic solvent (os), a compatible solute (cs), a crystallization solution (crs) and water, and wherein the ratio of volume compatible solute Vcs to volume organic solvent Vos is the same within a series of soaking solutions in said first dimension, or, alternatively, the molar ratio of organic solvent (os) to compatible solute (cs) Mos/Mcs is the same within a series of soaking solutions in said first dimension, and wherein the individual soaking solutions of said first dimension comprise the same organic solvent and the same compatible solute within a series of said soaking solutions in the first dimension, respectively, and wherein the ratio of the volume of water Vw and the volume of the crystallization solution Vcrs is varied over the individual soaking solutions of the series in the first dimension and wherein one of the individual soaking solutions of the series in the first dimension has a minimal or zero Vcrs and a maximum Vw and another individual soaking solution of said first dimension has a minimal or zero Vw and a maximum Vcrs and wherein the other individual soaking solutions of the series in said first dimension take values of Vcrs and Vw in between the two before-mentioned values.

17. A method for obtaining the solutions as claimed in claim 12, by an array that comprises a first dimension of at least two individual soaking solutions (1n to xn) and a second dimension of at least two individual soaking solutions (1m to ym) wherein each of said soaking solutions is located in a separated compartment of said array, and wherein each of said soaking solution comprises an organic solvent (os), a compatible solute (cs), a crystallization solution (crs) and water, and wherein the ratio of volume compatible solute Vcs to volume organic solvent Vos is the same within a series of soaking solutions in said first dimension, or, alternatively, the molar ratio of organic solvent (os) to compatible solute (cs) Mos/Mcs is the same within a series of soaking solutions in said first dimension, and wherein the individual soaking solutions of said first dimension comprise the same organic solvent and the same compatible solute within a series of said soaking solutions in the first dimension, respectively, and wherein the ratio of the volume of water Vw and the volume of the crystallization solution Vcrs is varied over the individual soaking solutions of the series in the first dimension and wherein one of the individual soaking solutions of the series in the first dimension has a minimal or zero Vcrs and a maximum Vw and another individual soaking solution of said first dimension has a minimal or zero Vw and a maximum Vcrs and wherein the other individual soaking solutions of the series in said first dimension take values of Vcrs and Vw in between the two before-mentioned values.
